(19) United States
(12) Patent Application Publication
(10) Pub. No.: US 2025/0277058 A1
(43) Pub. Date: Sep. 4, 2025

(54) ANTIBODIES THAT BIND GLUCOSAMINIDASE AND USES THEREOF

(71) Applicant: TELEPHUS BIOSCIENCES, LLC, Rochester, NY (US)

(72) Inventor: Mark BENEDYK, La Jolla, CA (US)

(21) Appl. No.: 18/264,895

(22) PCT Filed: Feb. 11, 2022
(86) PCT No.: PCT/US2022/016060

§ 371 Date: Aug. 9, 2023

Related U.S. Application Data
(60) Provisional application No. 63/149,003, filed on Feb. 12, 2021.

(51) Int. Cl. C07K 16/40

(52) U.S. Cl. CPC C07K 16/40; C07K 2317/24; C07K 2317/52; C07K 2317/565; C07K 2317/567; C07K 2317/73; C07K 2317/732; C07K 2317/92

(57) ABSTRACT

This disclosure provides glucosaminidase (Gmd)-binding antibodies capable of inhibiting growth of Staphylococcus and methods of making and using these antibodies in for example, diagnostic prognostic methods, and therapeutic applications. The disclosure further provides methods for treating a Staphylococcus (e.g., S. aureus) infection using the Gmd-binding antibodies. Methods for treating conditions associated with microbial infections including for example, fever, bacteremia, endocarditis, osteomyelitis, pneumonia, sepsis, septic shock, dermonecrosis, and mastitis are also provided.

### BACKGROUND

Staphylococcus aureus is a leading cause of mortality and morbidity worldwide, causing a diverse array of diseases by various pathogenic mechanisms and a variety of infections ranging from mild skin and soft-tissue infections to serious invasive diseases such as endocarditis, osteomyelitis, and necrotizing pneumonia (Lowy F., N. Engl. J. Med. 339(8):520-32 (1998); Klevens et al., JAMA 298(15):1763-71 (2007)). The growing prevalence of antimicrobial-resistant strains in both hospital and community settings has inhibited the choice of therapy and therapeutic success considerably, resulting in increased rates of chronic and recurrent infections and rising healthcare costs.

Accordingly, alternative methods for the prevention and treatment of bacterial infections in general and S. aureus infections in particular are needed.

### BRIEF SUMMARY

The disclosure provides glucosaminidase (Gmd)-binding antibodies (e.g., full length antibodies and Gmd-binding antibody fragments) capable of inhibiting growth of Staphylococcus and methods of making and using these antibodies. In some embodiments, the disclosure provides a method for treating a Staphylococcus (e.g., S. aureus) infection that comprises administering a therapeutically effective amount of a Gmd-binding antibody to a subject having or at risk of having a Staphylococcus infection. In some embodiments, the infection is a skin, soft-tissue, or invasive infection. In further embodiments, the disclosure provides a method for treating a condition associated with a Staphylococcus infection. In some embodiments, the condition associated with the Staphylococcus infection is selected from the group consisting of: fever, bacteremia, endocarditis, osteomyelitis, pneumonia, sepsis, septic shock, dermonecrosis, and mastitis. In some embodiments, the Staphylococcus is resistant to an antibmicrobial agent. In some embodiments, the Staphylococcus infection is an S. aureus infection. In some embodiments, the S. aureus is methicillin sensitive (MSSA). In additional embodiments, the S. aureus is resistant to an antimicrobial agent. In some embodiments, the S. aureus is resistant to one or more β-lactam antimicrobial agents. In some embodiments, the S. aureus is methicillin resistant (MRSA). Methods of detecting and diagnosing or prognosing a Staphylococcus (e.g., S. aureus) infection using the disclosed Gmd-binding antibodies are also provided.

In some embodiments, the Gmd-binding antibody is a full length antibody or a Gmd-binding antibody fragment. In additional embodiments, the Gmd-binding antibody cross-blocks or competes for binding to Gmd with a reference Gmd-binding antibody having a VH of SEQ ID NO:9 and VL of SEQ ID NO:41, but does not comprise the VH sequence, the VL sequence, or the VH sequence and the VL sequence of the reference antibody. In further embodiments, the Gmd-binding antibody binds to the same epitope of Gmd as a reference Gmd-binding antibody having a VH sequence of SEQ ID NO:9 and a VL sequence of SEQ ID NO:41, but does not comprise the VH sequence, the VL sequence, or the VH sequence and the VL sequence of the reference antibody. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) of S. aureus (e.g., Xen29); (d) promotes cell-independent lysis of S. aureus; (e) decreases binary fission or inhibits cell division of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%.

In additional embodiments, the Gmd-binding antibody (e.g., a full length antibody or a Gmd-binding antibody fragment) comprises a set of CDRs: VH-CDR1, VH-CDR2, VH-CDR3, VL-CDR1, VL-CDR2, and VL-CDR3, wherein the set of CDRs has a total of one, two, three, four, five, six, seven, eight, nine, ten, or fewer than ten, amino acid substitutions, deletions, and/or insertions from a reference set of CDRs in which: (a) (i) VH-CDR1 comprises the amino acid sequence of SEQ ID NO: 10; (ii) VH-CDR2 comprises the amino acid sequence of SEQ ID NO:11; (iii) VH-CDR3 comprises the amino acid sequence of SEQ ID NO:12; (iv) VL-CDR1 comprises the amino acid sequence of SEQ ID NO:42; (v) VL-CDR2 comprises the amino acid sequence of SEQ ID NO:43; and (vi) VL-CDR3 comprises the amino acid sequence of SEQ ID NO:44. In particular embodiments, the Gmd-binding antibody comprises a set of CDRs: wherein the (i) VH-CDR1 does not comprise the amino acid sequence of SEQ ID NO:10; (ii) VH-CDR2 does not comprise the amino acid sequence of SEQ ID NO:11; (iii) VH-CDR3 does not comprise the amino acid sequence of SEQ ID NO:12; (iv) VL-CDR1 does not comprise the amino acid sequence of SEQ ID NO:42; (v) VL-CDR2 does not comprise the amino acid sequence of SEQ ID NO:43; or (vi) VL-CDR3 does not comprise the amino acid sequence of SEQ ID NO:44.

In some embodiments, the Gmd-binding antibody (e.g., a full length antibody or a Gmd-binding antibody fragment) comprises a VH comprising a VH-CDR1 of SEQ ID NO:10, a VH-CDR2 of SEQ ID NO:11, and a VH-CDR3 of SEQ ID NO:12, and VL comprising a VH-CDR1 of SEQ ID NO: 42, a VH-CDR2 of SEQ ID NO:43, and a VH-CDR3 of SEQ ID NO:44, and comprises a framework sequence selected from the group consisting of (a) a VH framework 1 sequence of SEQ ID NO: 13 or 14; (b) a VH framework 2 sequence of SEQ ID NO:16-18, or 19; (c) a VH framework 3 sequence of SEQ ID NO:21; (d) a VL framework 1 sequence of SEQ ID NO:45, 46, or 47; (e) a VL framework 2 sequence of SEQ ID NO:49, 50, or 51; and (f) a VL framework 3 sequence of SEQ ID NO: 53-58, or 59. In some embodiments, the Gmd-binding antibody comprises a VH framework 3 sequence of SEQ ID NO:21. In some embodiments, the Gmd-binding antibody comprises a VH framework 3 sequence of SEQ ID NO:21. In some embodiments, the Gmd-binding antibody comprises a VH of SEQ ID NO:1-7, or 8. In further embodiments, the Gmd-binding antibody comprises a VL framework 4 sequence of SEQ ID NO:61.

In some embodiments, the Gmd-binding antibody (e.g., a full length antibody or a Gmd-binding antibody fragment) comprises a VH comprising a VH-CDR1 of SEQ ID NO:10, a VH-CDR2 of SEQ ID NO: 11, and a VH-CDR3 of SEQ ID NO:12, and VL comprising a VL-CDR1 of SEQ ID NO: 42, a VL-CDR2 of SEQ ID NO:43, and a VL-CDR3 of SEQ ID NO:44, and comprises a VH framework sequence selected from the group consisting of (a) a framework 1 sequence of SEQ ID NO: 13 and a framework 2 sequence of SEQ ID NO: 16; (b) a framework 1 sequence of SEQ ID NO:13 and a framework 2 sequence of SEQ ID NO: 17; (c) a framework 1 sequence of SEQ ID NO:13 and a framework 2 sequence of SEQ ID NO:18; (d) a framework 1 sequence of SEQ ID NO:13 and a framework 2 sequence of SEQ ID NO:19; (e) a framework 1 sequence of SEQ ID NO:14 and a framework 2 sequence of SEQ ID NO:16; (f) a framework 1 sequence of SEQ ID NO:14 and a framework 2 sequence of SEQ ID NO: 17; and (g) a framework 1 sequence of SEQ ID NO:14 and a framework 2 sequence of SEQ ID NO:18. In further embodiments, the Gmd-binding antibody comprises a VH framework 4 sequence of SEQ ID NO:24. In some embodiments, the Gmd-binding antibody comprises a VH of SEQ ID NO:1-7, or 8. In further embodiments, the Gmd-binding antibody comprises a VL framework 4 sequence of SEQ ID NO:61. In some embodiments, the Gmd-binding antibody comprises a VH framework 3 sequence of SEQ ID NO:21. In some embodiments, the Gmd-binding antibody comprises a VH of SEQ ID NO:1-7, or 8.

In some embodiments, the Gmd-binding antibody (e.g., a full length antibody or a Gmd-binding antibody fragment) comprises a VH comprising a VH-CDR1 of SEQ ID NO:10, a VH-CDR2 of SEQ ID NO:11, and a VH-CDR3 of SEQ ID NO:12, and VL comprising a VL-CDR1 of SEQ ID NO: 42, a VL-CDR2 of SEQ ID NO:43, and a VL-CDR3 of SEQ ID NO:44, and comprises a VH framework 1 sequence of SEQ ID NO: 13 and a framework 2 sequence of SEQ ID NO:18. In some embodiments, the Gmd-binding antibody comprises a VH comprising a VH-CDR1 of SEQ ID NO:10, a VH-CDR2 of SEQ ID NO: 11, and a VH-CDR3 of SEQ ID NO: 12, and VL comprising a VL-CDR1 of SEQ ID NO:42, a VL-CDR2 of SEQ ID NO:43, and a VL-CDR3 of SEQ ID NO:44, and comprises a VH framework 2 sequence of SEQ ID NO:18 and a framework 3 sequence of SEQ ID NO:21.

In some embodiments, the Gmd-binding antibody (e.g., a full length antibody or a Gmd-binding antibody fragment) comprises a VH comprising a VH-CDR1 of SEQ ID NO:10, a VH-CDR2 of SEQ ID NO: 11, and a VH-CDR3 of SEQ ID NO: 12, and VL comprising a VL-CDR1 of SEQ ID NO: 42, a VL-CDR2 of SEQ ID NO:43, and a VL-CDR3 of SEQ ID NO:44, and comprises a VH framework 1 sequence of SEQ ID NO:13 and a framework 2 sequence of SEQ ID NO:19. In some embodiments, the Gmd-binding antibody comprises a VH comprising a VH-CDR1 of SEQ ID NO:10, a VH-CDR2 of SEQ ID NO:11, and a VH-CDR3 of SEQ ID NO:12, and VL comprising a VL-CDR1 of SEQ ID NO:42, a VL-CDR2 of SEQ ID NO:43, and a VL-CDR3 of SEQ ID NO:44, and comprises a VH framework 2 sequence of SEQ ID NO: 19 and a framework 3 sequence of SEQ ID NO:21.

In some embodiments, the Gmd-binding antibody (e.g., a full length antibody or a Gmd-binding antibody fragment) comprises a VH comprising a VH-CDR1 of SEQ ID NO:10, a VH-CDR2 of SEQ ID NO: 11, and a VH-CDR3 of SEQ ID NO: 12, and VL comprising a VH-CDR1 of SEQ ID NO: 42, a VH-CDR2 of SEQ ID NO:43, and a VH-CDR3 of SEQ ID NO:44, and comprises a VH framework 3 sequence of SEQ ID NO:21. In further embodiments, the Gmd-binding antibody comprises a VH framework 4 sequence of SEQ ID NO:24. In some embodiments, the Gmd-binding antibody comprises a VH of SEQ ID NO:1-7, or 8.

In some embodiments, the Gmd-binding antibody (e.g., a full length antibody or a Gmd-binding antibody fragment) comprises a VH comprising a VH-CDR1 of SEQ ID NO:10, a VH-CDR2 of SEQ ID NO:11, and a VH-CDR3 of SEQ ID NO: 12, and VL comprising a VH-CDR1 of SEQ ID NO: 42, a VH-CDR2 of SEQ ID NO:43, and a VH-CDR3 of SEQ ID NO:44, and comprises a VL framework sequence selected from the group consisting of: (a) a framework 1 sequence of SEQ ID NO: 45 and a framework 2 sequence of SEQ ID NO:49; (b) a framework 1 sequence of SEQ ID NO:46 and a framework 2 sequence of SEQ ID NO:50; (c) a framework 1 sequence of SEQ ID NO:47 and a framework 2 sequence of SEQ ID NO:50; (d) a framework 1 sequence of SEQ ID NO:47 and a framework 2 sequence of SEQ ID NO:51; (e) a framework 2 sequence of SEQ ID NO:49 and a framework 3 sequence of SEQ ID NO:53; (f) a framework 2 sequence of SEQ ID NO:49 and a framework 3 sequence of SEQ ID NO:55; (g) a framework 2 sequence of SEQ ID NO:50 and a framework 3 sequence of SEQ ID NO:54; (h) a framework 2 sequence of SEQ ID NO:50 and a framework 3 sequence of SEQ ID NO:55; (i) a framework 2 sequence of SEQ ID NO:50 and a framework 3 sequence of SEQ ID NO:56; (j) a framework 2 sequence of SEQ ID NO:50 and a framework 3 sequence of SEQ ID NO:57; and (k) a framework 2 sequence of SEQ ID NO:51 and a framework 3 sequence of SEQ ID NO:58. In some embodiments, the Gmd-binding antibody comprises a VH framework 3 sequence of SEQ ID NO:21. In some embodiments, the Gmd-binding antibody comprises a VH of SEQ ID NO:1-7, or 8.

In some embodiments, the Gmd-binding antibody (e.g., a full length antibody or a Gmd-binding antibody fragment) comprises a VH comprising a VH-CDR1 of SEQ ID NO:10, a VH-CDR2 of SEQ ID NO:11, and a VH-CDR3 of SEQ ID NO: 12, and VL comprising a VH-CDR1 of SEQ ID NO: 42, a VH-CDR2 of SEQ ID NO:43, and a VH-CDR3 of SEQ ID NO:44, and comprises a VL having a framework 1 sequence of SEQ ID NO:45 and a framework 2 sequence of SEQ ID NO:49. In some embodiments, the Gmd-binding antibody comprises a VH comprising a VH-CDR1 of SEQ ID NO: 10, a VH-CDR2 of SEQ ID NO:11, and a VH-CDR3 of SEQ ID NO:12, and VL comprising a VH-CDR1 of SEQ ID NO:42, a VH-CDR2 of SEQ ID NO:43, and a VH-CDR3 of SEQ ID NO:44, and comprises a VL having a framework 2 sequence of SEQ ID NO:49 and a framework 3 sequence of SEQ ID NO:55.

In some embodiments, the Gmd-binding antibody (e.g., a full length antibody or a Gmd-binding antibody fragment) comprises a VH comprising a VH-CDR1 of SEQ ID NO:10, a VH-CDR2 of SEQ ID NO:11, and a VH-CDR3 of SEQ ID NO:12, and VL comprising a VH-CDR1 of SEQ ID NO: 42, a VH-CDR2 of SEQ ID NO:43, and a VH-CDR3 of SEQ ID NO:44, and comprises VL framework sequences selected from the group consisting of: (a) a framework 1 sequence of SEQ ID NO: 45 and a framework 2 sequence of SEQ ID NO:49; (b) a framework 1 sequence of SEQ ID NO:46 and a framework 2 sequence of SEQ ID NO:50; (c) a framework 1 sequence of SEQ ID NO:47 and a framework 2 sequence of SEQ ID NO:50; (d) a framework 1 sequence of SEQ ID NO:47 and a framework 2 sequence of SEQ ID NO:51; (e) a framework 2 sequence of SEQ ID NO:49 and a framework 3 sequence of SEQ ID NO:53; (f) a framework 2 sequence of SEQ ID NO:49 and a framework 3 sequence of SEQ ID NO:55; (g) a framework 2 sequence of SEQ ID NO:50 and a framework 3 sequence of SEQ ID NO:54; (h) a framework 2 sequence of SEQ ID NO:50 and a framework 3 sequence of SEQ ID NO:55; (i) a framework 2 sequence of SEQ ID NO:50 and a framework 3 sequence of SEQ ID NO:56; (j) a framework 2 sequence of SEQ ID NO:50 and a framework 3 sequence of SEQ ID NO:57; and (k) a framework 2 sequence of SEQ ID NO:51 and a framework 3 sequence of SEQ ID NO:58. In some embodiments, the Gmd-binding antibody comprises a VH framework 3 sequence of SEQ ID NO:21. In some embodiments, the Gmd-binding antibody comprises a VH of SEQ ID NO:1-7, or 8.

In some embodiments, the Gmd-binding antibody (e.g., a full length antibody or a Gmd-binding antibody fragment) comprises a Gmd-binding antibody which comprises a VH having at least 90%, 95%, 96%, 97%, 98%, 99%, or 100% sequence identity to SEQ ID NO: 1-7 or 8 and a VL having at least 90%, 95%, 96%, 97%, 98%, 99%, or 100% sequence identity to SEQ ID NO:33-39, or 40; and wherein the Gmd-binding antibody does not have a VH of SEQ ID NO:9 and/or a VL of SEQ ID NO: 41.

In some embodiments, the Gmd-binding antibody (e.g., a full length antibody or a Gmd-binding antibody fragment) comprises a VH and a VL pair selected from the group consisting of: (a) (i) a VH having at least 90%, 95%, 96%, 97%, 98%, 99%, or 100% sequence identity to SEQ ID NO:1, and a VL having at least 90%, 95%, 96%, 97%, 98%, 99%, or 100% sequence identity to SEQ ID NO: 34 or 35; (b) a VH having at least 90%, 95%, 96%, 97%, 98%, 99%, or 100% sequence identity to SEQ ID NO:2, and a VL having at least 90%, 95%, 96%, 97%, 98%, 99%, or 100% sequence identity to SEQ ID NO:34, 35, or 37; (c) a VH having at least 90%, 95%, 96%, 97%, 98%, 99%, or 100% sequence identity to SEQ ID NO:3 and a VL having at least 90%, 95%, 96%, 97%, 98%, 99%, or 100% sequence identity to SEQ ID NO:33, 35, 36, 38, or 39; (d) a VH having at least 90%, 95%, 96%, 97%, 98%, 99%, or 100% sequence identity to SEQ ID NO:4, and a VL having at least 90%, 95%, 96%, 97%, 98%, 99%, or 100% sequence identity to SEQ ID NO:39; (e) a VH having at least 90%, 95%, 96%, 97%, 98%, 99%, or 100% sequence identity to SEQ ID NO:5, and a VL having at least 90%, 95%, 96%, 97%, 98%, 99%, or 100% sequence identity to SEQ ID NO:35, 36, 38, or 39; (f) a VH having at least 90%, 95%, 96%, 97%, 98%, 99%, or 100% sequence identity to SEQ ID NO: 6, and a VL having at least 90%, 95%, 96%, 97%, 98%, 99%, or 100% sequence identity to SEQ ID NO: 35, 36, or 38; (g) a VH having at least 90%, 95%, 96%, 97%, 98%, 99%, or 100% sequence identity to SEQ ID NO:7, and a VL having at least 90%, 95%, 96%, 97%, 98%, 99%, or 100% sequence identity to SEQ ID NO:34 or 35; (h) a VH having at least 90%, 95%, 96%, 97%, 98%, 99%, or 100% sequence identity to SEQ ID NO:8, and a VL having at least 90%, 95%, 96%, 97%, 98%, 99%, or 100% sequence identity to SEQ ID NO:33-38, or 40; but does not comprise the VH sequence of SEQ ID NO:9, the VL sequence of SEQ ID NO:41, or the VH sequence of SEQ ID NO:9 and the VL sequence of SEQ ID NO:41.

In one embodiment, the Gmd-binding antibody (e.g., a full length antibody or a Gmd-binding antibody fragment) comprises a VH and a VL pair having a VH of SEQ ID NO:1-7 or 8 and a VL of SEQ ID NO:33-39, or 40.

In one embodiment, the Gmd-binding antibody (e.g., a full length antibody or a Gmd-binding antibody fragment) comprises a VH and a VL selected from the group consisting of: (a) a VH sequence of SEQ ID NO: 1 and a VL sequence of SEQ ID NO:34; (b) a VH sequence of SEQ ID NO:1 and a VL sequence of SEQ ID NO:35; (c) a VH sequence of SEQ ID NO:2 and a VL sequence of SEQ ID NO: 34; (d) a VH sequence of SEQ ID NO:2 and a VL sequence of SEQ ID NO:35; (e) a VH sequence of SEQ ID NO:3 and a VL sequence of SEQ ID NO:33; (f) a VH sequence of SEQ ID NO:3 and a VL sequence of SEQ ID NO:35; (g) a VH sequence of SEQ ID NO:3, and a VL sequence of SEQ ID NO:36; and (h) a VH sequence of SEQ ID NO:3 and a VL sequence of SEQ ID NO:38; (i) a VH sequence of SEQ ID NO:3 and a VL sequence of SEQ ID NO:39; (j) a VH sequence of SEQ ID NO: 4 and a VL sequence of SEQ ID NO:39; (k) a VH sequence of SEQ ID NO:5 and a VL sequence of SEQ ID NO:35; (l) a VH sequence of SEQ ID NO:5 and a VL sequence of SEQ ID NO:36; (m) a VH sequence of SEQ ID NO:5 and a VL sequence of SEQ ID NO:38; (n) a VH sequence of SEQ ID NO: 5 and a VL sequence of SEQ ID NO:39; (0) a VH sequence of SEQ ID NO:6, and a VL sequence of SEQ ID NO:35; (p) a VH sequence of SEQ ID NO:6 and a VL sequence of SEQ ID NO:36; (q) a VH sequence of SEQ ID NO:6 and a VL sequence of SEQ ID NO:38; (r) a VH sequence of SEQ ID NO: 7 and a VL sequence of SEQ ID NO:34; (s) a VH sequence of SEQ ID NO:7 and a VL sequence of SEQ ID NO:35; (t) a VH sequence of SEQ ID NO:8 and a VL sequence of SEQ ID NO:33; (u) a VH sequence of SEQ ID NO:8 and a VL sequence of SEQ ID NO:34; (v) a VH sequence of SEQ ID NO: 8 and a VL sequence of SEQ ID NO:35; (w) a VH sequence of SEQ ID NO:8, and a VL sequence of SEQ ID NO:36; (x) a VH sequence of SEQ ID NO:8 and a VL sequence of SEQ ID NO:37; (y) a VH sequence of SEQ ID NO:8 and a VL sequence of SEQ ID NO:38; and (2) a VH sequence of SEQ ID NO: 8 and a VL sequence of SEQ ID NO:40.

In one embodiment, the Gmd-binding antibody (e.g., a full length antibody or a Gmd-binding antibody fragment) comprises a VH and a VL containing a VH of SEQ ID NO:1, and a VL of SEQ ID NO:33-39, or 40. In one embodiment, the Gmd-binding antibody comprises a VH of SEQ ID NO:1 and a VL of SEQ ID NO:34 or 35. In one embodiment, the Gmd-binding antibody comprises a VH of SEQ ID NO:1 and a VL of SEQ ID NO:34. In a further embodiment, the Gmd-binding antibody comprises a VH of SEQ ID NO: 1 and a VL sequence of SEQ ID NO:35.

In another embodiment, the Gmd-binding antibody comprises a VH of SEQ ID NO:2 and a VL of SEQ ID NO:33-39, or 40. In one embodiment, the Gmd-binding antibody comprises a VH of SEQ ID NO:2 and a VL of SEQ ID NO:34, 35, or 37. In one embodiment, the Gmd-binding antibody comprises a VH of SEQ ID NO:2 and a VL of SEQ ID NO:34. In another embodiment, the Gmd-binding antibody comprises a VH of SEQ ID NO:2 and a VL of SEQ ID NO:35. In a further embodiment, the Gmd-binding antibody comprises a VH of SEQ ID NO:2 and a VL of SEQ ID NO:37.

In an additional further embodiment, the Gmd-binding antibody (e.g., a full length antibody or a Gmd-binding antibody fragment) comprises a VH of SEQ ID NO:3 and a VL of SEQ ID NO:33-39, or 40. In one embodiment, the Gmd-binding antibody comprises a VH of SEQ ID NO:3 and a VL of SEQ ID NO:33, 35, 36, 38, or 39. In one embodiment, the Gmd-binding antibody comprises a VH of SEQ ID NO:3 and a VL of SEQ ID NO:33. In another embodiment, the Gmd-binding antibody comprises a VH of SEQ ID NO:3 and a VL of SEQ ID NO:35. In another embodiment, the Gmd-binding antibody comprises a VH of SEQ ID NO:3 and a VL of SEQ ID NO:36. In another embodiment, the Gmd-binding antibody comprises a VH of SEQ ID NO:3 and a VL of SEQ ID NO:38. In a further embodiment, the Gmd-binding antibody comprises a VH of SEQ ID NO:3 and a VL of SEQ ID NO:39. In a further embodiment, the Gmd-binding antibody comprises a VH of SEQ ID NO:3 and a VL of SEQ ID NO:40.

In a further embodiment, the Gmd-binding antibody (e.g., a full length antibody or a Gmd-binding antibody fragment) comprises a VH of SEQ ID NO:4 and a VL of SEQ ID NO:33-39, or 40. In a further embodiment, the Gmd-binding antibody (e.g., a full length antibody or a Gmd-binding antibody fragment) comprises a VH and a VL containing a VH of SEQ ID NO:4, and a VL of SEQ ID NO: 39.

In a further embodiment, the Gmd-binding antibody (e.g., a full length antibody or a Gmd-binding antibody fragment) comprises a VH of SEQ ID NO:5 and a VL of SEQ ID NO:33-39, or 40. In one embodiment, the Gmd-binding antibody comprises a VH of SEQ ID NO:5 and a VL of SEQ ID NO: 35, 36, 38, or 39. In one embodiment, the Gmd-binding antibody comprises a VH of SEQ ID NO: 5 and a VL of SEQ ID NO:35. In another embodiment, the Gmd-binding antibody comprises a VH of SEQ ID NO:5 and a VL of SEQ ID NO:36. In another embodiment, the Gmd-binding antibody comprises a VH of SEQ ID NO:5 and a VL of SEQ ID NO:38. In a further embodiment, the Gmd-binding antibody comprises a VH of SEQ ID NO:5 and a VL of SEQ ID NO:39.

In an additional embodiment, the Gmd-binding antibody (e.g., a full length antibody or a Gmd-binding antibody fragment) comprises a VH of SEQ ID NO:6 and a VL of SEQ ID NO:33-39, or 40. In one embodiment, the Gmd-binding antibody comprises a VH of SEQ ID NO:6 and a VL of SEQ ID NO:35, 36, or 38. In one embodiment, the Gmd-binding antibody comprises a VH of SEQ ID NO: 6 and a VL of SEQ ID NO:35. In another embodiment, the Gmd-binding antibody comprises a VH of SEQ ID NO:6 and a VL of SEQ ID NO:36. In a further embodiment, the Gmd-binding antibody comprises a VH of SEQ ID NO:6 and a VL of SEQ ID NO:38.

In a further embodiment, the Gmd-binding antibody (e.g., a full length antibody or a Gmd-binding antibody fragment) comprises a VH of SEQ ID NO:7 and a VL of SEQ ID NO:33-39, or 40. In one embodiment, the Gmd-binding antibody comprises a VH of SEQ ID NO:7 and a VL of SEQ ID NO: 34 or 35. In one embodiment, the Gmd-binding antibody comprises a VH of SEQ ID NO:7 and a VL of SEQ ID NO:34. In a further embodiment, the Gmd-binding antibody comprises a VH of SEQ ID NO:7 and a VL of SEQ ID NO:35.

In another embodiment, the Gmd-binding antibody (e.g., a full length antibody or a Gmd-binding antibody fragment) comprises a VH of SEQ ID NO:8 and a VL of SEQ ID NO:33-39, or 40. In one embodiment, the Gmd-binding antibody comprises a VH of SEQ ID NO:3 and a VL of SEQ ID NO: 33-38, or 40. In one embodiment, the Gmd-binding antibody comprises a VH of SEQ ID NO:8 and a VL of SEQ ID NO:33. In another embodiment, the Gmd-binding antibody comprises a VH of SEQ ID NO:8 and a VL of SEQ ID NO:34. In another embodiment, the Gmd-binding antibody comprises a VH of SEQ ID NO:8 and a VL of SEQ ID NO:35. In another embodiment, the Gmd-binding antibody comprises a VH of SEQ ID NO:8 and a VL of SEQ ID NO:36. In another embodiment, the Gmd-binding antibody comprises a VH of SEQ ID NO:8 and a VL of SEQ ID NO:37. In another embodiment, the Gmd-binding antibody comprises a VH of SEQ ID NO:8 and a VL of SEQ ID NO:38.

In some embodiments, the Gmd-binding antibody (e.g., a full length antibody or a Gmd-binding antibody fragment) comprises (a) a VH sequence having a total of 1, 2, 3, 4, 5, 6, 7, 8, 9, 10, fewer than 15, or zero, amino acid substitutions, deletions, and/or insertions from a reference VH sequence of SEQ ID NO: 1-7 or 8, and (b) a VL sequence having a total of 1, 2, 3, 4, 5, 6, 7, 8, 9, 10, fewer than 15, or zero, amino acid substitutions, deletions, and/or insertions from a reference VL sequence of SEQ ID NO:33-39, or 40; and wherein the Gmd-binding antibody does not comprise the VH sequence of SEQ ID NO:9, the VL sequence of SEQ ID NO:41, or the VH sequence of SEQ ID NO: 9 and the VL sequence of SEQ ID NO:41. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) of S. aureus (e.g., Xen29); (d) promotes cell-independent lysis of S. aureus; (e) decreases binary fission or cell division of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%.

In some embodiments, the Gmd-binding antibody (e.g., a full length antibody or a Gmd-binding antibody fragment) comprises a VH and a VL pair selected from the group consisting of: (a) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:1, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:33-39, or 40; (b) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:2, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; (c) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:3, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; (d) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:4, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; (e) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:5, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; (f) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:6, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; (g) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:7, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; and (h) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:8, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; and wherein the Gmd-binding antibody does not comprise the VH sequence of SEQ ID NO:9, the VL sequence of SEQ ID NO:41, or the VH sequence of SEQ ID NO:9 and the VL sequence of SEQ ID NO:41.

In some embodiments, the Gmd-binding antibody (e.g., a full length antibody or a Gmd-binding antibody fragment) comprises a VH and a VL pair selected from the group consisting of: (a) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:1, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:34 or 35; (b) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:2, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:34, 35, or 37; (c) a VH having sequence having a total of 1, 2, 3, 4, 5, 6, 7, 8, 9, 10, fewer than 15, or zero, amino acid substitutions, deletions, and/or insertions from a reference VH sequence of SEQ ID NO:3, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:33, 35, 36, 38, or 39; (d) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:4, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:39; (e) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:5, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:35, 36, 38, or 39; (f) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:6, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:35, 36, or 38; (g) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:7, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:34 or 35; and (h) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:8, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:33-38 or 40; and wherein the Gmd-binding antibody does not comprise the VH sequence of SEQ ID NO:9, the VL sequence of SEQ ID NO:41, or the VH sequence of SEQ ID NO:9 and the VL sequence of SEQ ID NO: 41.

In some embodiments, the Gmd-binding antibody (e.g., a full length antibody or a Gmd-binding antibody fragment) comprises a VH sequence having a total of 1, 2, 3, 4, 5, 6, 7, 8, 9, 10, fewer than 15, or zero, amino acid substitutions, deletions, and/or insertions from a reference VH sequence of SEQ ID NO:7.

In some embodiments, the Gmd-binding antibody (e.g., a full length antibody or a Gmd-binding antibody fragment) comprises a VL sequence having a total of 1, 2, 3, 4, 5, 6, 7, 8, 9, 10, fewer than 15, or zero, amino acid substitutions, deletions, and/or insertions from a reference VL sequence of SEQ ID NO:34.

In some embodiments, the Gmd-binding antibody (e.g., a full length antibody or a Gmd-binding antibody fragment) comprises a VL sequence having a total of 1, 2, 3, 4, 5, 6, 7, 8, 9, 10, fewer than 15, or zero, amino acid substitutions, deletions, and/or insertions from a reference VL sequence of SEQ ID NO:35.

In some embodiments, the Gmd-binding antibody (e.g., a full length antibody or a Gmd-binding antibody fragment) comprises a VH sequence having a total of 1, 2, 3, 4, 5, 6, 7, 8, 9, 10, fewer than 15, or zero, amino acid substitutions, deletions, and/or insertions from a reference VH sequence of SEQ ID NO:7 and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:34.

In some embodiments, the Gmd-binding antibody (e.g., a full length antibody or a Gmd-binding antibody fragment) comprises a VH sequence having a total of 1, 2, 3, 4, 5, 6, 7, 8, 9, 10, fewer than 15, or zero, amino acid substitutions, deletions, and/or insertions from a reference VH sequence of SEQ ID NO:7 and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:35.

In some embodiments, the Gmd-binding antibody is a monoclonal antibody, a recombinant antibody, a human antibody, a humanized antibody, a chimeric antibody, a bi-specific antibody, or a multi-specific antibody. In some embodiments, the Gmd-binding antibody is a full length antibody. In some embodiments, the Gmd-binding antibody is a Gmd-binding antibody fragment. In further embodiments, the Gmd-binding antibody is an antibody fragment selected from the group consisting of a Fab, Fab′, F(ab′)₂, Fv, diabody, DART, and a single chain antibody molecule (e.g., a BiTE).

In some embodiments, the Gmd-binding antibody (e.g., a full length antibody or a Gmd-binding antibody fragment) binds a Gmd fragment consisting of the amino acid sequence of SEQ ID NO: 72. In further embodiments, the Gmd-binding antibody binds a Gmd fragment consisting of the amino acid sequence of amino acid residues 59-91 of SEQ ID NO:72.

Nucleic acids and sets of nucleic acids encoding Gmd-binding antibodies (e.g., full length antibodies and Gmd-binding antibody fragments) are also provided. Vectors and sets of vectors containing these nucleic acids and host cells transformed with these nucleic acids and vectors are further provided. In some embodiments, the host cell is a hybridoma or mammalian host cell such as, a NS0 murine myeloma cell, a PER.C6® human cell, or a Chinese hamster ovary (CHO) cell. Host cells including mammalian host cells and hybridomas that produce Gmd-binding antibodies are also provided.

Methods for making Gmd-binding antibodies are also provided. In some embodiments, the method comprises culturing a host cell capable of expressing the Gmd-binding antibody (e.g., a full length antibody or a Gmd-binding antibody fragment) under suitable conditions for expressing the protein and optionally isolating the expressed Gmd-binding antibody. Gmd-binding antibodies prepared and/or isolated using methods disclosed herein or otherwise known in the art are also provided.

Pharmaceutical compositions comprising a Gmd-binding antibody (e.g., a full length antibody or a Gmd-binding antibody fragment) and a pharmaceutically acceptable carrier are further provided.

In some embodiments, the disclosure provides a method for treating a Staphylococcus (e.g., S. aureus) infection in a subject having or at risk of having a Staphylococcus infection. In further embodiments, the Staphylococcus is S. aureus. In some embodiment, the method decreases the Staphylococcus titer in the subject. In further embodiments, the disclosure provides a method for treating a condition associated with a Staphylococcus infection in a patient. Conditions that may be treated in a subject using the provided methods include but are not limited to fever, fever, bacteremia, endocarditis, osteomyelitis, pneumonia, sepsis, dermonecrosis, and mastitis.

In some embodiments, the provided methods include administering a pharmaceutical composition comprising an effective amount of a Gmd-binding antibody disclosed herein to a subject in need thereof. In some embodiments, the Gmd-binding antibody is administered alone. In additional embodiments, the Gmd-binding antibody is administered as a combination therapy. In some embodiments, the Gmd-binding antibody is administered as a combination therapy with an antibiotic. In some embodiments, the Gmd-binding antibody is administered as a combination therapy to the standard of care treatment/therapy.

The disclosure also provides a method of inhibiting the growth of Staphylococcus (e.g., S. aureus) that comprises contacting a Gmd-binding antibody (e.g., a full length antibody or a Gmd-binding antibody fragment) and Staphylococcus. In some embodiments, Staphylococcus is S. aureus. In some embodiments, the method of inhibiting the growth of Staphylococcus is performed in vitro. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In some embodiments, the method is performed in vivo. In some embodiments, the Gmd-binding antibody inhibits in vivo growth of S. aureus by 20% to 100%, 30% to 100%, 40% to 100%, 50% to 100%, 60% to 100%, 70% to 100%, or 80% to 100%. In some embodiments, the method comprises contacting a Gmd-binding antibody and an antibiotic-resistant Staphylococcus (e.g., S. aureus). In some embodiments, the method comprises contacting a Gmd-binding antibody and a methicillin-susceptible S. aureus. In some instances the method comprises contacting a Gmd-binding antibody and a methicillin-susceptible S. aureus (MSSA). In some instances the method comprises contacting a Gmd-binding antibody and a methicillin-resistant S. aureus (MRSA). In some instances the method comprises contacting a Gmd-binding antibody and a vancomycin resistant S. aureus (VRSA). In some instances the method comprises contacting a Gmd-binding antibody and a daptomycin-resistant S. aureus (DRSA). In some instances the method comprises contacting a Gmd-binding antibody and a linezolid-resistant S. aureus (LRSA). In some instances the method comprises contacting a Gmd-binding antibody and a bacteria with altered antibiotic sensitivity such as vancomycin intermediate-sensitivity S. aureus (VISA). In some embodiments, the disclosure provides a method of inhibiting growth of Staphylococcus in a subject that comprises administering an effective amount of a Gmd-binding antibody to a subject in need thereof.

In some embodiments, the disclosure provides a method for treating a Staphylococcus infection that comprises administering an effective amount of a Gmd-binding antibody (e.g., a full length antibody or a Gmd-binding antibody comprising the sequence of a VH and VL pair disclosed in Table 1) to a patient having or at risk of having a Staphylococcus infection. In further embodiments, the disclosure provides a method for treating a condition associated with the Staphylococcus infection. In some embodiments, the condition associated with the infection is selected from the group consisting of fever, bacteremia, endocarditis, osteomyelitis, pneumonia, sepsis, dermonecrosis, and maspatientis.

In further embodiments, the disclosure provides a method for treating an S. aureus infection that comprises administering an effective amount of a Gmd-binding antibody (e.g., a full length antibody or a Gmd-binding antibody fragment comprising the sequence of a VH and VL pair disclosed in Table 1) to a patient having or at risk of having a S. aureus infection. In further embodiments, the disclosure provides a method for treating a condition associated with the S. aureus infection. In some embodiments, the condition associated with the infection is selected from the group consisting of fever, bacteremia, endocarditis, osteomyelitis, pneumonia, sepsis, dermonecrosis, and maspatientis. In some embodiments, the S. aureus is resistant to one or more β-lactam antimicrobials. In some embodiments, the S. aureus is a member selected from the group consisting of: VRSA, DRSA, LRSA, and VISA.

In some embodiments, the disclosure provides a method for treating a Staphylococcus (e.g., S. aureus) bone or joint infection that comprises administering to a patient having or at risk of having a bone or joint infection, an effective amount of a Gmd-binding antibody disclosed herein (e.g., a full length antibody and a Gmd-binding antibody fragment having a sequence disclosed in Table 1). In further embodiments, the disclosure provides a method for treating osteomyelitis that comprises administering to a patient having or at risk of having a Staphylococcus bone or joint infection an effective amount of a Gmd-binding antibody a Gmd-binding antibody (e.g., a full length antibody or a Gmd-binding antibody comprising the sequence of a VH and VL pair disclosed in Table 1). In some embodiments, the infection is an S. aureus infection. In some embodiments, the S. aureus is MSSA. In additional embodiments, the S. aureus is resistant to an antibiotic. In some embodiments, the S. aureus is MRSA. In some embodiments, the S. aureus is resistant to one or more β-lactam antimicrobials. In some embodiments, the S. aureus is a member selected from the group consisting of: VRSA, DRSA, LRSA, and VISA.

In some embodiments, the disclosure provides a method for preventing infection while treating hard or soft tissue, that comprises administering to a patient undergoing or about to undergo treatment of hard or soft tissue (e.g., surgery, etc) an effective amount of a Gmd-binding antibody disclosed herein (e.g., a full length antibody and a Gmd-binding antibody fragment having a sequence disclosed in Table 1) or otherwise disclosed herein.

In some embodiments, the disclosure provides a method for treating hard or soft tissue in the presence of an infection (e.g., S. aureus), that comprises administering to a patient undergoing or about to undergo treatment of infected hard or soft tissue (e.g., surgery, etc) an effective amount of a Gmd-binding antibody disclosed herein (e.g., a full length antibody and a Gmd-binding antibody fragment having a sequence disclosed in Table 1) or otherwise disclosed herein.

In additional embodiments, the disclosure provides a method of introducing a medical device (e.g., an orthopedic implant, dental implant, or ENT implant) or tissue graft into a patient that comprises administering to a patient in need of an medical device an effective amount of a Gmd-binding antibody disclosed herein (e.g., a full length antibody and a Gmd-binding antibody fragment), and introducing the orthopedic implant, tissue graft, or medical device into the patient. In some embodiments, the Gmd-binding antibody is administered to the patient before introducing the medical device (e.g., orthopedic implant). In some embodiments, the Gmd-binding antibody is administered to the patient at the same time as introducing the medical device. In some embodiments, the Gmd-binding antibody is administered to the patient after the medical device is introduced. In some embodiments, the Gmd-binding antibody is administered to the patient before and after the medical device is introduced into the patient.

In additional embodiments, the disclosure provides a method of diagnosing or prognosing a Staphylococcus (e.g., S. aureus) infection that comprises contacting a patient sample with a Gmd-binding antibody disclosed herein, and detecting the presence of an immune complex between the Gmd-binding antibody and S. aureus or Gmd present in the sample, whereby the detection of the immune complex indicates the presence of Staphylococcus in the patient sample. In further embodiments, the patient sample is from the blood, serum, or plasma of a patient.

In another embodiment, the disclosure provides a method of determining the presence of Staphylococcus (e.g., S. aureus) in a sample that comprises contacting a sample with a Gmd-binding antibody disclosed herein, and detecting the presence of an immune complex formed between the Gmd-binding antibody and Staphylococcus or Gmd protein in the sample, whereby the detection of the immune complex indicates the presence of Staphylococcus in the sample. In further embodiments, the sample is a biological sample (e.g., a sample containing or derived from the blood, serum, or plasma of a subject).

### DETAILED DESCRIPTION

The disclosure provides isolated and/or recombinant Gmd-binding antibodies (e.g., full length antibodies and Gmd-binding antibody fragments). Nucleic acids encoding the Gmd-binding antibodies, vectors and host cells containing the nucleic acids, and methods of making and using the Gmd-binding antibodies are also provided. The provided Gmd-binding antibodies have uses in diagnosing and treating diseases and conditions associated with a Staphylococcus infection. Such uses include but are not limited to treating a Staphylococcus (e.g., S. aureus) infection and conditions associated with a Staphylococcus infection such as, fever, bacteremia, endocarditis, osteomyelitis, pneumonia, sepsis, dermonecrosis, and maspatientis.

### Definitions

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. The headings provided herein are not limitations of the various embodiments, which can be had by reference to the specification as a whole. Moreover, the terms defined immediately below are more fully defined by reference to the specification in its entirety.

The terms “a,” “an” and “the” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B.” “A or B,” “A” (alone), and “B” (alone). Likewise, the term “and/or” as used in a phrase such as “A, B, and/or C” is intended to encompass each of the following embodiments: a, B, and C; A. B., or C; A or C; A or B; B or C; A and C; A and B; B and C; A (alone); B (alone); and C (alone).

The term “comprise” or “comprises” is generally used in the sense of include, that is to say permitting the presence of one or more features or components. Wherever embodiments, are described herein with the language “comprises” or “comprising,” otherwise analogous embodiments, described in terms of “consisting of,” and/or “consisting essentially of” are also provided.

The terms “about” and “approximately” as used in connection with a numerical value throughout the specification and the claims denotes an interval of accuracy, familiar and acceptable to a person skilled in the art. In general such interval of accuracy is ±10%. Alternatively, and particularly in biological systems, the terms “about” and “approximately” may mean values that are within an order of magnitude, preferably ≤5-fold and more preferably ≤2-fold of a given value.

Numeric ranges are inclusive of the numbers defining the range.

The terms “Gmd,” “endo-β-N-acetylglucosaminidase,” and “glucosaminidase” are used interchangeably herein to refer to the S. aureus glucosaminidase enzyme. Gmd is greater than 95% conserved across all strains of S. aureus in the public database, and approximately 85% conserved among other staphylococci. In particular embodiments, the Gmd has the amino acid sequence of:

(SEQ ID NO: 71)
AYTVTKPQTTQTVSKIAQVKPNNTGIRASVYEKTAKNGAKYADRTFYV
TKERAHGNETYVLLNNTSHNIPLGWFNVKDLNVQNLGKEVKTTQKYTV
NKSNNGLSMVPWGTKNQVILTGNNIAQGTFNATKQVSVGKDVYLYGTI
NNRTGWVNAKDLTAPTAVKPTTSAAKDYNYTYVIKNGNGYYYVTPNSD
TAKYSLKAFNEQPFAVVKEQVINGQTWYYGKLSNGKLAWIKSTDLAKE
LIKYNQTGMTLNQVAQIQAGLQYKPQVQRVPGKWTDANFNDVKHAMDT
KRLAQDPALKYQFLRLDQPQNISIDKINQFLKGKGVLENQGAAFNKAA
QMYGINEVYLISHALLETGNGTSQLAKGADVVNNKVVTNSNTKYHNVF
GIAAYDNDPLREGIKYAKQAGWDTVSKAIVGGAKFIGNSYVKAGQNTL
YKMRWNPAHPGTHQYATDVDWANINAKIIKGYYDKIGEVGKYFDIPQY
K.

In further embodiments, the R3 domain of Gmd has the amino acid sequence of: QTTQTVSKIAQVKPNNTGIRASVYEKTAKNGAKYADRTFYVTKERAHGNETYVLLNNTSHNIPLGW FNVKDLNVQNLGKEVKTTQKYTVNKSNNGLSMVPWGTKNQVILTGNNIAQGTFNATKQVSVGKD VYLYGTI (SEQ ID NO: 72), corresponding to amino acid residues 8-144 of S. aureus Gmd (SEQ ID NO: 71). In some embodiments, the Gmd-binding antibody disclosed herein binds to the R3 domain of S. aureus Gmd (SEQ ID NO:71) with an affinity greater than 10⁻⁸ M or 10⁻⁹ M, but preferably greater than 10⁻¹⁰ M. In some embodiments, the Gmd-binding antibody binds to a polypeptide consisting of the amino acid sequence of SEQ ID NO:72 with an affinity greater than 10⁻⁸ M or 10⁻⁹ M, but preferably greater than 10⁻¹⁰ M. In some embodiments, the Gmd-binding antibody binds to a polypeptide consisting amino acids 60-92 of SEQ ID NO:72 with an affinity greater than 10⁻⁸ M or 10⁻⁹ M, but preferably greater than 10⁻¹⁰ M.

The term “compete” or “competes” when used in the context of Gmd-binding antibodies (e.g., full length antibodies and Gmd-binding antibody fragments) means competition between the Gmd-binding proteins as determined by an assay in which the Gmd-binding antibody or a Gmd-binding antibody fragment under test prevents or inhibits specific binding of a reference Gmd binding protein (e.g., a ligand, or a reference antibody) to a common antigen (e.g., Gmd or a fragment thereof such as the R3 domain (SEQ ID NO:72). Numerous types of competitive binding assays can be used to determine whether two proteins compete for binding to a common antigen, for example: solid phase direct or indirect radioimmunoassay (RIA) (see, e.g., Moldenhauer et al., Scand. J. Immunol. 32:77-82 (1990) and Morel et al., Molec. Immunol. 25:7-15 (1988)), solid phase direct or indirect enzyme immunoassay (EIA), solid phase direct biotin-avidin EIA (see, e.g., Cheung, et al., Virology 176:546-552 (1990) and Kirkland et al., J. Immunol. 137:3614-3619 (1986)) and a sandwich competition assay (see, e.g., Stahli et al., Methods in Enzymology 92:242-253 (1983)). Typically, such an assay involves the use of purified antigen bound to a solid surface or cells bearing either of these, an unlabeled test antigen binding protein and a labeled reference antigen binding protein.

Competitive inhibition can be measured by determining the amount of label bound to the solid surface or cells in the presence of the test antigen binding protein. Usually the test antigen binding protein is present in excess. Antigen binding proteins identified by competition assay (competing antigen binding proteins) include Gmd-binding antibodies that bind to the same epitope as the reference Gmd-binding antibody as well as Gmd-binding antibodies that bind to an adjacent epitope sufficiently proximal to the epitope bound by the reference Gmd-binding antibody for steric hindrance to occur. Usually, when a competing Gmd-binding antibody is present in excess, it will inhibit specific binding of a reference Gmd-binding antibody to Gmd by at least 40%, 45%, 50%, 55%, 60%, 65%, 70% or 75%. In some instance, a competing Gmd-binding antibody inhibits specific binding of a reference Gmd-binding antibody by at least 80%, 85%, 90%, 91%, 92%, 93%, 94%, 95%, 96%, 97% 98%, or 99%.

The term “epitope” when used in context of a Gmd protein refers to a Gmd (e.g., a Gmd fragment consisting of the amino acid sequence of SEQ ID NO:72 or a Gmd having the amino acid sequence of amino acid residues 59-91 of SEQ ID NO:72) protein determinant capable of binding to a Gmd-binding antibody of the disclosure. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. The Gmd epitope bound by a Gmd-binding antibody can readily be determined using techniques known in the art.

The anti-Gmd-binding antibodies disclosed herein, can be described or specified in terms of the epitope(s) or portion(s) of Gmd, e.g., a target polypeptide that they recognize or specifically bind. For example, the portion of Gmd that specifically interacts with the antigen binding domain of a Gmd-binding antibody disclosed herein is an “epitope.” Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. Epitope determinants may include chemically active surface groupings of molecules such as amino acids, sugar side chains, phosphoryl or sulfonyl groups, and may have specific three dimensional structural characteristics, and/or specific charge characteristics. An epitope typically includes at least 3, 4, 5, 6, 3, 4, 5, 6, 7, 8, 9, 10, 11, 12, 13, 14, 15, 16, 17, 18, 19, 20, 25, 30, 35 amino acids in a unique spatial conformation. Epitopes can routinely be determined using methods known in the art.

The terms “inhibit.” “block,” “reduce,” “decrease,” “suppress,” “antagonize,” and “neutralize” are used interchangeably and refer to any statistically significant decrease in activity (e.g., the in vitro or in vivo growth or other activity of a microorganism that expresses Gmd (e.g., S. aureus) or the binding of a Gmd-binding antibody to Gmd), including full blocking of the activity. For example, “inhibition,” “suppression,” or “antagonize” can refer to a decrease of about 10%, 20%, 30%, 40%, 50%, 60%, 70%, 80%, 90% or 100% in activity compared to a control.

The terms “antibody” and “immunoglobulin,” are used interchangeably herein, and include whole (full-length) antibodies and antigen binding fragments or single chains thereof. A typical antibody comprises at least two heavy (H) chains and two light (L) chains interconnected by disulfide bonds. Each heavy chain is comprised of a heavy chain variable region (abbreviated herein as VH) and a heavy chain constant region. The heavy chain constant region is comprised of three domains, CHI, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed Complementarity Determining Regions (CDR), interspersed with regions that are more conserved, termed framework regions (FWs). Each VH and VL is composed of three CDRs and four FWs, arranged from amino-terminus to carboxy-terminus in the following order: FW1, CDR1, FW2, CDR2, FW3, CDR3, FW4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Exemplary antibodies include typical antibodies, scFvs, and combinations thereof where, for example, an scFv is covalently linked (for example, via peptidic bonds or via a chemical linker) to the N- or C-terminus of either the heavy chain and/or the light chain of a typical antibody, or intercalated in the heavy chain and/or the light chain of a typical antibody. In some embodiments, the heavy chain constant region does not have a carboxy terminal lysine residue.

The terms “antibody” and “immunoglobulin,” encompass intact polyclonal antibodies, intact monoclonal antibodies, antibody fragments (such as Fab, Fab′, F(ab′)₂, and Fv fragments), single chain Fv (scFv) derivatives and mutants, multispecific antibodies such as bispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired binding activity. An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc. The term “IgG” refers to a polypeptide belonging to the class of antibodies that are substantially encoded by a recognized immunoglobulin gamma gene. In humans this class comprises IgG1, IgG2, IgG3, and IgG4. In mice this class comprises IgG1, IgG2a, IgG2b, and IgG3.

The terms “Gmd-binding antibody.” “anti-Gmd antibody” or “antibody that binds to Gmd,” refer to an antibody that is capable of binding Gmd with sufficient affinity such that the Gmd-binding antibody is useful as a therapeutic agent or diagnostic reagent in targeting Gmd, respectively.

By “specifically binds” when used in the context of Gmd proteins, it is generally meant the ability of a binding protein such as an antibody, to bind to Gmd (e.g., S. aureus Gmd preferably having the amino acid sequence of SEQ ID NO:72), with greater affinity than the Gmd-binding antibody binds to an unrelated control protein. In some embodiments, the control protein is hen egg white lysozyme. Preferably the Gmd-binding antibody binds Gmd with an affinity that is at least, 100, 500, or 1000 times greater than the affinity for a control protein. Preferably, the Gmd-binding antibody has a binding affinity for Gmd having the amino acid sequence of SEQ ID NO:72), of ≤1×10⁻⁷ M or ≤ 1×10⁻⁸ as measured using a binding assay known in the art. In some embodiments, the binding affinity is measured using a radioimmunoassay (RIA) or BIACORE® (e.g., using Gmd as the analyte and Gmd-binding antibody as the ligand, or vice versa).

In some embodiments, the extent of binding of a Gmd-binding antibody (e.g., a full length antibody or a Gmd-binding antibody fragment) to an unrelated, non-Gmd protein (e.g., albumin) is less than about 10% of the binding of the Gmd-binding antibody to Gmd as measured, for example, by a radioimmunoassay (RIA), BIACORE® (using recombinant Gmd as the analyte and Gmd-binding antibody as the ligand, or vice versa), kinetic exclusion assay (KINEXA®), or other binding assays known in the art. In certain embodiments, the Gmd-binding antibody is a full-length antibody or a Gmd-binding antibody fragment that has a dissociation constant (K_{D}) of ≤1 μM, ≤100 nM, ≤10 nM, ≤1 nM, ≤0.1 nM, ≤10 pM, ≤1 pM, or ≤0.1 pM (e.g., as determined by BIACORE® analysis).

The term “Gmd-binding antibody fragment” refers to a fragment containing all or a portion of an antigen binding variable region (e.g., CDR3) of an intact antibody. It is known that the antigen binding function of an antibody can be performed by fragments of a full-length antibody. Examples of antibody fragments include, but are not limited to Fab, Fab′, F(ab′)₂, and Fv fragments, linear antibodies, single chain antibodies, and multispecific antibodies formed from one or more antibody fragments. In some embodiments, the Gmd-binding antibody fragment is a Fab fragment, a Fab′ fragment, an F(ab′)₂ fragment, an Fv fragment, a diabody, or a single chain antibody molecule.

The “Fc” region includes polypeptides comprising the constant region of an antibody excluding the first constant region immunoglobulin domain. Thus, Fc refers to the last two constant region immunoglobulin domains of IgA. IgD, and IgG, and the last three constant region immunoglobulin domains of IgE and IgM, and the flexible hinge N-terminal to these domains. For IgA and IgM Fc may include the J chain. For IgG, Fc comprises immunoglobulin domains Cγ2 and Cγ3 and the hinge between Cγ1 and Cγ2. Although the boundaries of the Fc region may vary, the human IgG heavy chain Fc region is usually defined to comprise residues C226 or P230 to its carboxyl-terminus, wherein the numbering is according to the EU index as set forth in Kabat (Kabat et al., Sequences of Proteins of Immunological Interest, 5th Ed. Public Health Service, NIH, Bethesda, Md. (1991)). Fc may refer to this region in isolation, or this region in the context of a whole antibody, antibody fragment, or Fc fusion protein. Polymorphisms have been observed at a number of different Fc positions, including but not limited to positions 270, 272, 312, 315, 356, and 358 as numbered by the EU index, and thus slight differences between the presented sequence and sequences in the prior art may exist.

A “monoclonal antibody” refers to a homogeneous antibody population involved in the highly specific recognition and binding of a single antigenic determinant or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)₂, and Fv), single chain (scFv) mutants, and fusion proteins) comprising an antibody portion, and any other modified immunoglobulin molecule comprising an antigen recognition site. A monoclonal antibody may be made in any number of ways including, but not limited to, by hybridoma, phage selection, recombinant expression, and transgenic animals.

The term “chimeric antibody” refers to an antibody wherein the amino acid sequence of the immunoglobulin molecule is derived from two or more species. Typically, the variable region of both light and heavy chains corresponds to the variable region of antibodies derived from one species of mammal (e.g., mouse, rat, rabbit, etc.) with the desired antigen-binding specificity, affinity, and/or capability while the constant regions are homologous to the sequences in antibodies derived from another species (usually human) to avoid eliciting an immune response in that species.

The term “humanized antibody” refers to an antibody derived from a non-human (e.g., murine) immunoglobulin, which has been engineered to contain fewer preferably minimal non-human (e.g., murine) sequences. Typically, humanized antibodies are human immunoglobulins in which residues from the CDR are replaced by residues from the CDR of a non-human species (e.g., mouse, rat, rabbit, or hamster) that have the desired antigen-binding specificity, affinity, and/or capability (Jones, Nature 321:522-525 (1986); Riechmann, Nature 332:323-327 (1988); Verhoeyen, Science 239:1534-1536 (1988)). In some instances, the Fv framework region (FW) residues of a human immunoglobulin are replaced with the corresponding residues in an antibody from a non-human species that has the desired antigen-binding specificity, affinity, and/or capability. The humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody can also comprise at least a portion of an immunoglobulin constant region or domain (Fc), typically that of a human immunoglobulin. Examples of methods used to generate humanized antibodies are described in U.S. Pat. Nos. 5,225,539 and 5,639,641.

The term “human antibody” refers to an antibody produced by a human or an antibody having an amino acid sequence corresponding to an antibody produced by a human made using any technique known in the art. The term “human antibody” includes intact (full-length) antibodies, fragments thereof, and/or antibodies comprising at least one human heavy and/or light chain polypeptide such as, an antibody comprising murine light chain and human heavy chain polypeptides.

An “antagonist.” “blocking.” or “neutralizing” antibody is one that inhibits or reduces activity of the antigen it binds (e.g., Gmd) and/or an organism that expresses the antigen (e.g., S. aureus). In certain embodiments, the Gmd-binding antibody substantially or completely inhibits the activity of Gmd. In some embodiments, the Gmd activity is reduced by 10%, 20%, 30%, 50%, 70%, 80%, 90%, 95%, or 100%. In further embodiments, the antagonist Gmd-binding antibody inhibits or reduces the activity of Gmd by at least 10%, 20%, 30%, 50%, 70%, 80%, 90%, 95%, or even 100%. In some embodiments, the Gmd-binding antibody (e.g., a full length antibody or a Gmd-binding antibody fragment) reduces or inhibits in vitro growth of S. aureus. In some embodiments, the Gmd activity is reduced by 10%, 20%, 30%, 50%, 70%, 80%, 90%, 95%, or 100%. In further embodiments, the antagonist Gmd-binding antibody inhibits or reduces the activity of Gmd by at least 10%, 20%, 30%, 50%, 70%, 80%, 90%, 95%, or even 100%.

“Binding affinity” generally refers to the strength of the sum total of non-covalent interactions between a single binding site of a molecule (e.g., an antibody) and its binding partner (e.g., an antigen). Unless indicated otherwise, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein and can be used for the purposes of the present disclosure.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enables these cytotoxic effector cells to bind specifically to a Gmd expressing/bearing target cell and subsequently kill the target cell with cytotoxins. Specific high-affinity IgG antibodies directed to the surface of target cells “arm” the cytotoxic cells and are absolutely required for such killing. Lysis of the target cell is extracellular, requires direct cell-to-cell contact, and does not involve complement. It is contemplated that, in addition to antibodies, other proteins comprising Fc regions, specifically Fc fusion proteins, having the capacity to specifically bind to a target cell that expresses Gmd on its surface will be able to effect cell-mediated cytotoxicity. For simplicity, the cell-mediated cytotoxicity resulting from the activity of an Fc fusion protein is also referred to herein as ADCC activity.

A Gmd-binding antibody (e.g., a full length antibody and a Gmd-binding antibody fragment), polynucleotide, vector, cell, or composition which is “isolated” is a protein (e.g., antibody), polynucleotide, vector, cell, or composition which is in a form not found in nature. Isolated proteins, polynucleotides, vectors, cells or compositions include those which have been purified to a degree that they are no longer in a form in which they are found in nature. In some embodiments, a protein, polynucleotide, vector, cell, or composition which is isolated is substantially pure. Isolated proteins and isolated nucleic acid will be free or substantially free of material with which they are naturally associated such as other polypeptides or nucleic acids with which they are found in their natural environment, or the environment in which they are prepared (e.g., cell culture) when such preparation is by recombinant DNA technology practiced in vitro or in vivo. Proteins and nucleic acid may be formulated with diluents or adjuvants and still for practical purposes be isolated—for example, the proteins will normally be mixed with gelatin or other carriers if used to coat microtitre plates for use in immunoassays, or will be mixed with pharmaceutically acceptable carriers or diluents when used in diagnosis or therapy.

The terms “subject.” “individual,” “animal,” “patient,” and “mammal,” refer to any subject, particularly a mammalian subject, for whom diagnosis, prognosis, or therapy is desired. Mammalian subjects include but are not limited to humans, non-human primates, domestic animals, farm animals, rodents, and the like, which is to be the recipient of a particular treatment. The term “subject in need thereof” refers to any subject (preferably human) having or at risk of having a Staphylococcus (e.g., S. aureus) infection or a condition associated therewith.

The terms “controlling” or “reducing” with respect to bacterial growth or bacterial colonization refer to any measurable reduction or inhibition of bacterial growth or colonization and can be determined empirically and in a routine manner, in relation to the stated purpose.

An “effective amount” of a polypeptide, e.g., a Gmd-binding antibody, as disclosed herein is an amount sufficient to carry out a stated purpose. An “effective amount” can be determined empirically and in a routine manner, in relation to the stated purpose. The phrase “effective amount”, as used herein, generally refers to the amount of the antimicrobial coating applied to the implant in order to provide one or more clinically measurable endpoints, such as uncomplicated fracture healing or tissue remodeling in the presence of bacterial contamination or infection. The term “therapeutically effective amount” refers to an amount of a polypeptide, e.g., a Gmd-binding antibody or other drug effective to “treat” an infection (e.g., a condition associated with an infection) in a subject (e.g., a mammal such as a human) and provides some improvement or benefit to a subject having the infection or condition. Thus, a “therapeutically effective” amount is an amount that provides some alleviation, mitigation, and/or decrease in at least one clinical symptom or condition associated with a Staphylococcus (e.g., S. aureus) infection. Clinical symptoms associated with infections and conditions that can be treated using the disclosed Gmd-binding antibodies, pharmaceutical compositions containing the antibodies, and methods, are well known. Further, therapeutic effects need not be complete or curative, as long as some benefit is provided to the subject. In some embodiments, the term “therapeutically effective” refers to an amount of a therapeutic agent that is capable of reducing Gmd activity in a subject in need thereof. The actual amount administered and rate and time-course of administration, will depend on the nature and severity of the indication being treated. Prescription of treatment, e.g., decisions on dosage etc., is within the responsibility of general practitioners and other medical doctors. Appropriate doses of antibodies and antigen binding fragments thereof are generally known; see, Ledermann et al., Int. J. Cancer 47:659-664 (1991); Bagshawe et al., Ant. Immun. and Radiopharm. 4:915-922 (1991).

The terms “Staphylococcus,” “Staphylococcus strain,” and “Staphylococcus spp.” are used synonymously herein unless otherwise indicated. In some embodiments, the Staphylococcus is a strain that is, or can be, pathogenic to humans or animals. The Staphylococcus can be either coagulase-positive or coagulase-negative. Exemplary Staphylococcus strains include, without limitation, S. aureus, S. epidermidis, S. lugdunensis, S. saprophyticus, S. haemolyticus, S. caprae, and S. simiae. In some embodiments, the Gmd-binding antibodies provided herein are effective against an antibiotic-resistant strain of Staphylococcus. In some embodiments, the Gmd-binding antibodies are effective against S. aureus. In some embodiments, the S. aureus is methicillin sensitive (MSSA). In additional embodiments, the S. aureus is resistant to an antimicrobial agent. In some embodiments, the S. aureus is resistant to one or more β-lactam antimicrobial agents. In some embodiments, the S. aureus is methicillin resistant (MRSA). In some embodiments, the antibodies are effective against a methicillin-resistant strain. In some embodiments, the antibodies are effective against a vancomycin-resistant strain. Exemplary Staphylococcus strains that can be used according to the assays provided herein include, but are not limited to S. aureus subsp. Aureus Rosenbach (ATCC® 29213™; MSSA), S. aureus USA 100 (NSR382) MRSA; S. aureus USA 200 (NSR383) MRSA; S. aureus USA 300 (NSR384) MRSA; S. aureus Newman strain (NCTC 10833) MSSA; and S. aureus Smith strain (NCTC 10399) MSSA.

The term “pharmaceutical composition” refers to a preparation which is in such form as to permit the biological activity of the active ingredient to be effective, and which contains no additional components at concentrations that are unacceptably toxic to a subject to which the composition would be administered. Such composition can be sterile.

As used herein, the term “medical device” refers to any type of device/appliance that is totally or partly introduced, surgically or medically, into a patient's body and which may remain there after a procedure or may be removed during treatment. In some embodiments, the medical device is used for spine, trauma, or dental applications. In some embodiments, the medical device is an implant.

An “implant” is any medical device (object) intended for placement in the body of a subject (e.g., a mammal, such as a human) that is not a living tissue. Implants have uses that include orthopedic applications, dental applications, car, nose, and throat (“ENT”) applications, neurosurgical applications, and cardiovascular applications. Implants can be made of a variety of biocompatible materials, including: metals, ceramics, polymers, gels and fluids not normally found within the human body. Examples of polymers useful in fabricating medical devices/implants include such polymers as silicones, rubbers, latex, plastics, thermoplastics, polyanhydrides, polyesters, polyorthoesters, polyamides, polyacrylo-nitrile, polyurethanes, polyethylene, polytetrafluoroethylene, polyethylenetetraphthalate, polyphazenes, and fluoroplastics. Medical devices can also be fabricated using certain naturally-occurring materials or treated naturally-occurring materials. Implants can include any combination of artificial materials, combinations selected because of the particular characteristics of the components. For example, a hip implant can include a combination of a metallic shaft to bear the weight, a ceramic artificial joint and a polymeric glue to affix the structure to the surrounding bone. Implants can reside wholly in the body or partly in the body and partly outside the body. Implants can be intended for short-term or long-term residence where they are positioned.

In some embodiments, the implant is an orthopedic implant. An “orthopedic implant” is an implant which replaces bone or provides fixation to bone, replaces articulating surfaces of a joint, provides abutment for a prosthetic, or combinations thereof.

In some embodiments, the implant is a dental implant. In additional embodiments, the implant is an ENT implant. An ENT implant is an implant which restores structure and/or function to cars, nose, and/or throat. In other embodiments, the implant is a cosmetic implant. A cosmetic implant is an implant that provides tissue support for example dermal fillers or structural support such as chins for reconstructive craniomaxillofacial surgery.

As used herein, “introducing” a medical device is defined as introducing or installing the device or graft for the first time, as well as resurfacing or otherwise modifying a previously installed device or graft, replacing—in whole or in part—a previously installed device or graft, or otherwise surgically modifying a previously installed device or graft.

A “sufficient amount” or “an amount sufficient to” achieve a particular result in a subject (patient) having or at risk of having an infection or condition associated with an infection refers to an amount of a therapeutic agent (e.g., a Gmd-binding antibody) that is effective to produce a desired effect, which is optionally a therapeutic effect (i.e., by administration of a therapeutically effective amount). In some embodiments, such particular result is a reduction in Gmd activity in a subject in need thereof.

The term “label” refers to a detectable compound or composition which is conjugated directly or indirectly to a moiety such as a Gmd-binding antibody so as to generate a “labeled” moiety. The label can be detectable by itself (e.g., radioisotope labels or fluorescent labels) or, in the case of an enzymatic label, can catalyze chemical alteration of a substrate compound or composition which is detectable.

Terms such as “treating.” or “treatment.” “to treat” or “ameliorating” and “to ameliorate” refer to both (a) therapeutic measures that cure, slow down, lessen symptoms of, and/or halt progression of a diagnosed pathologic condition or disorder and (b) prophylactic or preventative measures that prevent and/or slow the development of a targeted infection or condition. Thus, subjects in need of treatment include those already with the infection or condition; those at risk of having the infection or condition; and those in whom the infection or condition is to be prevented. In certain embodiments, a subject is successfully “treated” according to the methods provided herein if the subject shows, e.g., total, partial, or transient amelioration or elimination of a symptom associated with the infection or condition. Conditions that can be treated with the Gmd-binding antibodies include, but are not limited to fever, bacteremia, endocarditis, osteomyelitis, pneumonia, sepsis, dermonecrosis, and mastitis. In some embodiments, treatment promotes or results in a decrease in the number of microbial cells or organisms (e.g., in a subject) relative to the number of microbial cells or organisms prior to treatment; a decrease in the viability (e.g., the average/mean viability) of microbial cells or organisms (e.g., in a subject) relative to the viability (e.g., the average/mean viability) of microbial cells or organisms (e.g., in the subject) prior to treatment; or reductions in one or more symptoms associated with one or more conditions in a subject relative to the subject's symptoms prior to treatment. In some embodiments, the disclosure provides a method for treating a Staphylococcus (e.g., S. aureus) infection. In further embodiments, the method treats a condition associated with a Staphylococcus (e.g., S. aureus) infection.

As used herein, “in combination with” or “combination therapies” refers to any form of administration such that additional therapies (e.g., second, third, fourth, etc.) are still effective in the body (e.g., multiple compounds are simultaneously effective in the subject, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject that receives such treatment can benefit from a combined effect of different therapies. One or more Gmd-binding antibodies (e.g., full length antibodies and Gmd-binding antibody fragments) provided herein can be administered concurrently with, prior to, or subsequent to, one or more other additional agents and/or supportive therapies. In general, each therapeutic agent will be administered at a dose and/or on a time schedule determined for that particular agent. The particular combination to employ in a regimen will take into account compatibility of the Gmd-binding antibody with therapy and/or the desired outcome.

The methods and techniques of the disclosure are generally performed according to known conventional methods and as described in various general and more specific references that are cited and discussed throughout the present disclosure unless otherwise indicated. See, e.g., Sambrook et al., Molecular Cloning: a Laboratory Manual, 3rd ed., Cold Spring Harbor Laboratory Press, Cold Spring Harbor, N.Y. (2001) and Ausubel et al., Current Protocols in Molecular Biology, Greene Publishing Associates (1992), and Harlow and Lane Antibodies: a Laboratory Manual Cold Spring Harbor Laboratory Press, Cold Spring Harbor, N.Y. (1990), all of which are herein incorporated by reference.

The terms “polynucleotide” and “nucleic acid” are used interchangeably and are intended to encompass a singular nucleic acid as well as plural nucleic acids, and refers to an isolated nucleic acid molecule(s) or construct(s), e.g., messenger RNA (mRNA), complementary DNA (cDNA), or plasmid DNA (pDNA) encoding the heavy chains and/or light chains of the Gmd-binding antibodies. In certain embodiments, a polynucleotide comprises a conventional phosphodiester bond or a non-conventional bond (e.g., an amide bond, such as found in peptide nucleic acids (PNA)). The term “nucleic acid” refers to any one or more nucleic acid segments, e.g., DNA, cDNA, or RNA fragments, present in a polynucleotide. When applied to a nucleic acid or polynucleotide, the term “isolated” refers to a nucleic acid molecule, DNA or RNA, which has been removed from its native environment, for example, a recombinant polynucleotide encoding a Gmd-binding antibody contained in a vector is considered isolated for the purposes of the present disclosure. Further examples of an isolated polynucleotide include recombinant polynucleotides maintained in heterologous host cells or purified (partially or substantially) from other polynucleotides in a solution. Isolated RNA molecules include in vivo or in vivo RNA transcripts of polynucleotides of the present disclosure. Isolated polynucleotides or nucleic acids according to the present disclosure further include such molecules produced synthetically. In addition, polynucleotides or nucleic acids can include regulatory elements such as promoters, enhancers, ribosome binding sites, or transcription termination signals.

The term “vector” means a construct, which is capable of delivering, and in some embodiments, expressing, one or more gene(s), coding sequence(s), or other sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The term “host cell” refers to a cell or a population of cells harboring or capable of harboring a recombinant nucleic acid. Host cells can be prokaryotic (e.g., E. coli), or eukaryotic. The host cells can be fungal cells including yeast such as Saccharomyces cerevisiae, Pichia pastoris, or Schizosaccharomyces pombe. The host cell can also be any of various animal cells, such as insect cells (e.g., Sf-9) or mammalian cells (e.g., HEK293F, CHO, COS-7, NIH-3T3, NS0, PER.C6®, and hybridoma). In further embodiments, the host cell is a CHO cell selected from the group consisting of CHO-K, CHO-0 CHO-Lec10, CHO-Lec13, CHO-Lec1, CHO Pro-5, and CHO dhfr. In particular embodiments, the host cell is a hybridoma.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because in some embodiments, the Gmd-binding antibodies are based upon Gmd-binding antibody fragments and can occur as single chains or associated chains.

A “recombinant” polypeptide, protein or antibody refers to polypeptide, protein or antibody produced via recombinant DNA technology. Recombinantly produced polypeptides, proteins and antibodies expressed in host cells are considered isolated for the purpose of the present disclosure, as are native or recombinant polypeptides which have been separated, fractionated, or partially or substantially purified by any suitable technique.

Also included in the present disclosure are fragments, variants, or derivatives of polypeptides, and any combination thereof. The term “fragment” when referring to polypeptides and proteins include any polypeptides or proteins which retain at least some of the properties of the reference polypeptide or protein. Fragments of polypeptides include proteolytic fragments, as well as deletion fragments.

The term “variant” refers to an antibody or polypeptide sequence that differs from that of a parent antibody or reference polypeptide sequence by virtue of at least one amino acid modification. Variants of antibodies or polypeptides include fragments, and also antibodies or polypeptides with altered amino acid sequences due to amino acid substitutions, deletions, or insertions. Variants can be naturally or non-naturally occurring. Non-naturally occurring variants can be produced using art-known mutagenesis techniques. Variant polypeptides can comprise conservative or non-conservative amino acid substitutions, deletions or additions.

The term “derivatives” as applied to antibodies or polypeptides refers to antibodies or reference parent polypeptides which have been altered so as to exhibit additional features not found on the native antibody or polypeptide. An example of a “derivative” antibody is a fusion or a conjugate with a second polypeptide or another molecule (e.g., a polymer such as PEG, a chromophore, or a fluorophore) or atom (e.g., a radioisotope).

The term “amino acid substitution” refers to replacing an amino acid residue present in a parent sequence with another amino acid residue. An amino acid can be substituted in a parent sequence, for example, via chemical peptide synthesis or through known recombinant methods. Accordingly, references to a “substitution at position X” or “substitution at position X” refer to the substitution of an amino acid residue present at position X with an alternative amino acid residue. In some embodiments, substitution patterns can described according to the schema AXY, wherein A is the single letter code corresponding to the amino acid residue naturally present at position X, and Y is the substituting amino acid residue. In other embodiments, substitution patterns can described according to the schema XY, wherein Y is the single letter code corresponding to the amino acid residue substituting the amino acid residue naturally present at position X.

A “conservative amino acid substitution” is one in which the amino acid residue is replaced with an amino acid residue having a similar side chain. Families of amino acid residues having similar side chains have been previously defined, including basic side chains (e.g., Lys, Arg, and His), acidic side chains (e.g., Asp and Glu), uncharged polar side chains (e.g., Gly, Asp, Gln, Ser, Thr, Tyr, and Cys), nonpolar side chains (e.g., Ala, Val, Leu, Ile, Pro, Phe, Met, and Trp), beta-branched side chains (e.g., Thr, Val, and Ile) and aromatic side chains (e.g., Tyr, Phe, Trp, and His). Thus, if an amino acid residue in a polypeptide is replaced with another amino acid residue from the same side chain family, the substitution is considered to be conservative. In another embodiment, a string of amino acid residues can be conservatively replaced with a structurally similar string that differs in order and/or composition of side chain family members.

Non-conservative substitutions include those in which (a) a residue having an electropositive side chain (e.g., Arg, His, or Lys) is substituted for, or by, an electronegative residue (e.g., Glu or Asp), (b) a hydrophilic residue (e.g., Ser or Thr) is substituted for, or by, a hydrophobic residue (e.g., Ala, Leu, Ile, Phe, or Val), (c) a Cys or Pro is substituted for, or by, any other residue, or (d) a residue having a bulky hydrophobic or aromatic side chain (e.g., Val, His, Ile, or Trp) is substituted for, or by, one having a smaller side chain (e.g., Ala or Ser) or no side chain (e.g., Gly).

Other substitutions can be readily identified. For example, for the amino acid alanine, a substitution can any one of: D-Ala, Gly, beta-Ala, L-Cys and D-Cys. For lysine, a replacement can be any one of D-Lys, Arg, D-Arg, homo-Arg. Met, D-Met, ornithine, or D-ornithine. Generally, substitutions in functionally important regions that can be expected to induce changes in the properties of the Gmd-binding antibody are those in which (a) a polar residue (e.g., Ser or Thr) is substituted for (or by) a hydrophobic residue (e.g., Leu, Ile, Phe, or Ala); (b) a Cys residue is substituted for (or by) any other residue; (c) a residue having an electropositive side chain (e.g., Lys, Arg, or His), is substituted for (or by) a residue having an electronegative side chain (e.g., Glu or Asp); or (d) a residue having a bulky side chain (e.g., Phe) is substituted for (or by) one not having such a side chain (e.g., Gly). The likelihood that one of the foregoing non-conservative substitutions can alter functional properties of the Gmd-binding antibody is also correlated to the position of the substitution with respect to functionally important regions of the protein: some non-conservative substitutions can accordingly have little or no effect on biological properties.

The term “amino acid insertion” refers to introducing an additional amino acid residue between two amino acid residues present in the parent sequence. An amino acid residue can be inserted in a parent sequence, for example, via chemical peptide synthesis or through recombinant methods known in the art. Accordingly, the phrases “insertion between positions X and Y” or “insertion between Kabat positions X and Y,” wherein X and Y correspond to amino acid residue positions (e.g., a cysteine amino acid residue insertion between positions 239 and 240), refers to the insertion of an amino acid residue between the X and Y positions, and also to the insertion in a nucleic acid sequence of a codon encoding an amino acid residue between the codons encoding the amino acid residues at positions X and Y.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence. The percentage of sequence identity is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences can be accomplished using readily available software programs. Suitable software programs are available from various sources, and for alignment of both protein and nucleotide sequences. One suitable program to determine percent sequence identity is bl2seq, part of the BLAST suite of program available from the U.S. government's National Center for Biotechnology Information BLAST web site (blast.ncbi.nlm.nih.gov). Bl2seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

The structure for carrying a CDR or a set of CDRs will generally be of an antibody heavy or light chain sequence or substantial portion thereof in which the CDR or set of CDRs is located at a location corresponding to the CDR or set of CDRs of naturally occurring VH and VL antibody variable domains encoded by rearranged immunoglobulin genes. The structures and locations of immunoglobulin variable domains and their CDRs can readily be determined by one skilled in the art using programs and known variable domain residue numbering systems such as Chothia, Chothia+, and Kabat can routinely be determined by reference to Kabat (Kabat et al., Sequences of Proteins of Immunological Interest. 4th Edition. U.S. DHHS. 1987, and tools available on the Internet (e.g., at bioinf.org.uk/abysis/sequence_input/key_annotation/key_annotation html; and immuno.bme. nwu.edu)), herein incorporated by reference in its entirety.

CDRs can also be carried by other scaffolds such as fibronectin, cytochrome B, albumin (e.g., ALBUdAb (Domantis/GSK) and ALB-Kunitz (Dyax)), unstructured repeat sequences of 3 or 6 amino acids (e.g., PASylation® technology and XTEN® technology), and sequences containing elastin-like repeat domains (see, e.g., U.S. Appl. No. 61/442,106, which is herein incorporated by reference in its entirety).

A CDR amino acid sequence substantially as set out herein can be carried as a CDR in a human variable domain or a substantial portion thereof. The HCDR3 sequences substantially as set out herein represent embodiments, of the present disclosure and each of these may be carried as a HCDR3 in a human heavy chain variable domain or a substantial portion thereof.

Variable domains employed in the present disclosure can be obtained from any germ-line or rearranged human variable domain, or can be a synthetic variable domain based on consensus sequences of known human variable domains. A CDR sequence (e.g., CDR3) can be introduced into a repertoire of variable domains lacking a CDR (e.g., CDR3), using recombinant DNA technology. For example, Marks et al., (Bio/Technology 10:779-783 (1992), which is herein incorporated by reference in its entirety) provide methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR3. Marks et al., further describe how this repertoire can be combined with a CDR3 of a particular antibody. Using analogous techniques, the CDR3-derived sequences of the present disclosure can be shuffled with repertoires of VH or VL domains lacking a CDR3, and the shuffled complete VH or VL domains combined with a cognate VH or VL domain to provide Gmd-binding antibodies. The repertoire can then be displayed in a suitable host system such as the phage display system of Intl. Appl. Publ. No. WO92/01047 or any of a subsequent large body of literature, including Kay et al., ((1996) Phage Display of Peptides and Proteins: a Laboratory Manual, San Diego: academic Press), so that suitable Gmd-binding antibodies may be selected. A repertoire can consist anything from 10⁴ individual members upwards, for example from 10⁶ to 10⁸, or 10¹⁰, members. Other suitable host systems include but are not limited to yeast display, bacterial display, T7 display, and ribosome display. For a review of ribosome display for see Lowe et al., Curr. Pharm. Biotech. 517-527 (2004) and Intl. Appl. Publ. No. WO92/01047, each of which is herein incorporated by reference in its entirety. Analogous shuffling or combinatorial techniques are also disclosed by Stemmer (Nature 370:389-391 (1994)), which is herein incorporated by reference in its entirety), which describes the technique in relation to a β-lactamase gene but observes that the approach may be used to generate antibodies.

A Gmd-binding antibody is said to “compete” with a reference molecule for binding to Gmd if it binds to Gmd to the extent that it blocks, to some degree, binding of the reference molecule to Gmd. The ability of a Gmd-binding antibody to compete for binding to Gmd and thus to interfere with, block or “cross-block” one another's binding to Gmd can be determined by any standard competitive binding assay known in the art including, for example, a competition ELISA assay, surface plasmon resonance (SPR; BIACORE®, Biosensor, Piscataway, N.J.) or according to methods described by Scatchard et al. (Ann. N.Y. Acad. Sci. 51:660-672 (1949)) or otherwise known in the art. A Gmd-binding antibody may be said to competitively inhibit binding of the reference molecule to Gmd, for example, by at least 90%, at least 80%, at least 70%, at least 60%, or at least 50%. According to some embodiments, the Gmd-binding antibody competitively inhibits binding of the reference molecule to Gmd, by at least 90%, at least 80%, at least 70%, at least 60%, or at least 50%. According to other embodiments, the Gmd-binding antibody competitively inhibits binding of a reference molecule to Gmd, by at least 90%, at least 80%, at least 70%, at least 60%, or at least 50%.

### Gmd-Binding Antibodies

In some embodiments, the Gmd-binding antibody (e.g., a full length antibody or a Gmd-binding antibody fragment) specifically binds a Gmd fragment consisting of the amino acid sequence of SEQ ID NO:72 (e.g., a fragment consisting of amino acid residues 59-91 of SEQ ID NO:72) with an affinity that is at least, 100, 500, or 1000 times greater than the affinity of the Gmd-binding antibody for a control protein that is not Gmd (e.g., human serum albumin). In certain embodiments, the Gmd-binding antibody binds Gmd and has a dissociation constant (K_{D}) of <1 μM, <100 nM, <10 nM, <1 nM, <0.1 nM, <10 pM, <1 pM, or <0.1 pM (e.g., as determined by BIACORE® analysis). In some embodiments, the Gmd-binding antibody has a K_{D} for Gmd having the sequence of SEQ ID NO:72 within the range of ≤1 μM and ≥0.1 pM, ≤100 μM and ≥0.1 pM, or ≤100 μM and ≥1 pM (e.g., as determined by BIACORE® analysis). In some embodiments, the Gmd-binding antibody has a K_{D} for Gmd having the sequence of SEQ ID NO:71 within the range of ≤1 μM and ≥0.1 pM, ≤100 μM and ≥0.1 pM, or ≤100 μM and ≥1 pM (e.g., as determined by BIACORE® analysis).

The affinity or avidity of a Gmd-binding antibody (e.g., a full length antibody or a Gmd-binding antibody fragment) for Gmd can be determined experimentally using any suitable method known in the art, e.g., flow cytometry, enzyme-linked immunosorbent assay (ELISA), or radioimmunoassay (RIA), or kinetics (e.g., BIACORE® or KINEXA® analysis). Direct binding assays and competitive binding assay formats can be readily employed. (See, for example, Berzofsky et al., “Antibody-Antigen Interactions,” In Fundamental Immunology, Paul, W. E., Ed., Raven Press: new York, N.Y. (1984); Kuby, Immunology, W. H. Freeman and Company: new York, N.Y. (1992); and methods described herein.) The measured affinity of a particular antibody-antigen interaction can vary if measured under different conditions (e.g., salt concentration, pH, temperature). Thus, measurements of affinity and other Gmd-binding parameters (e.g., K_{D} or Kd, Kₒₙ, K_{off}) are made with standardized solutions of Gmd-binding antibodies (e.g., full length antibodies and Gmd-binding antibody fragments) and Gmd and the measurements are performed using standardized conditions and methods, as described herein or otherwise known in the art.

In some embodiments, BIACORE® analysis is used to determine the ability of a Gmd-binding antibody (e.g., a full length antibody or a Gmd-binding antibody fragment) to compete with/block the binding to Gmd protein by a reference Gmd-binding antibody. In some embodiments, the ability of a Gmd-binding antibody to compete with/block the binding to Gmd protein by a reference Gmd-binding antibody is determined using a BIACORE® instrument (for example the BIACORE® 3000) operated according to the manufacturer's recommendations, wherein Gmd-Fc fusion protein is captured on a CM5 BIACORE® chip by previously attached anti-niFc IgG to generate a Gmd (e.g., SEQ ID NO:72)-coated surface. Typically 200-800 resonance units of Gmd-Fc (dimeric) would be coupled to the chip (an amount that gives easily measurable levels of binding but that are readily saturable by the concentrations of test reagent being used).

In another embodiment, the two Gmd-binding antibodies (e.g., full length antibodies and/or Gmd-binding antibody fragments) (termed A* and B*) to be assessed for their ability to compete with/block each other are mixed at a one to one molar ratio of binding sites in a suitable buffer to create a test mixture. When calculating the concentrations on a binding site basis the molecular weight of a Gmd-binding antibody is assumed to be the total molecular weight of the Gmd-binding antibody divided by the number of Gmd-binding sites on that Gmd-binding antibody. The concentration of each Gmd-binding antibody (i.e., A* and B*) in the test mixture should be high enough to readily saturate the binding sites for that Gmd-binding antibody on the Gmd-Fc molecules captured on the BIACORE® chip. The A* and B* Gmd-binding antibodies in the mixture are at the same molar concentration (on a binding basis) and that concentration would typically be between 1.00 and 1.5 micromolar (on a binding site basis). Separate solutions containing Gmd-binding antibody A* alone and Gmd-binding antibody B* alone are also prepared. Gmd-binding antibody A* and Gmd-binding antibody B* in these solutions should be in the same buffer and at the same concentration as in the test mixture. The test mixture is passed over the Gmd-Fc-coated BIACORE® chip and the total amount of binding recorded. The chip is then treated in such a way as to remove the bound Gmd-binding antibodies without damaging the chip-bound Gmd-Fc. Typically, this is done by treating the chip with 30 mM HCl for 60 seconds. The solution of Gmd-binding antibody A* alone is then passed over the Gmd-Fc-coated surface and the amount of binding recorded. The chip is again treated to remove the bound antibody without damaging the chip-bound Gmd-Fc. The solution of Gmd-binding antibody B* alone is then passed over the Gmd-Fc-coated surface and the amount of binding recorded. The maximum theoretical binding of the mixture of Gmd-binding antibody A* and Gmd-binding antibody B* is next calculated, and is the sum of the binding of each Gmd-binding antibody when passed over the Gmd surface alone. If the actual recorded binding of the mixture is less than this theoretical maximum then the two Gmd-binding antibodies are competing with/blocking each other. Thus, in general, a blocking Gmd-binding antibody is one which will bind to Gmd in the above BIACORE® blocking assay such that during the assay and in the presence of a second Gmd-binding antibody the recorded binding is between 80% and 0.1% (e.g., 80% to 4%) of the maximum theoretical binding, specifically between 75% and 0.1% (e.g., 75% to 4%) of the maximum theoretical binding, and more specifically between 70% and 0.1% (e.g., 70% to 4%) of maximum theoretical binding (as defined above) of the two Gmd-binding antibodies in combination.

The BIACORE® assays described above are exemplary assays used to determine if two Gmd-binding proteins (e.g., full length antibodies) compete with/block each other for binding Gmd. On rare occasions, particular Gmd-binding antibodies may not bind to Gmd-Fc coupled via anti-Fc IgG to a CM5 BIACORE® chip (for example, this might occur when the relevant binding site on Gmd is masked or destroyed by Gmd linkage to Fc). In such cases, blocking can be determined using a tagged version of Gmd, for example C-terminal His-tagged Gmd. In this particular format, an anti-His antibody would be coupled to the BIACORE® chip and then the His-tagged Gmd would be passed over the surface of the chip and captured by the anti-His antibody. The cross-blocking analysis would be carried out essentially as described above, except that after each chip regeneration cycle, new His-tagged Gmd would be loaded back onto the surface coated with anti-His antibody. Moreover, various other known tags and tag binding protein combinations can be used for such a blocking analysis (e.g., HA tag with anti-HA antibodies; FLAG tag with anti-FLAG antibodies; biotin tag with streptavidin). The following generally describes an ELISA assay for determining whether a Gmd-binding antibody blocks or is capable of blocking the binding of a reference Gmd-binding antibody to Gmd.

In some embodiments, an ELISA is used to determine the ability of a Gmd-binding antibody (e.g., a full length antibody) to compete for binding to Gmd with a reference Gmd-binding antibody (e.g., a Gmd binding antibody disclosed herein). The general principle of such an assay is to have a reference Gmd-binding antibody (coated onto the wells of an ELISA plate. An excess amount of a second potentially blocking, test Gmd-binding antibody is added in solution (i.e., not bound to the ELISA plate). A limited amount of Gmd (or alternatively Gmd-Fc) is then added to the wells. The coated reference Gmd-binding antibody and the test Gmd-binding antibody in solution compete for binding of the limited number of Gmd (or Gmd-Fc) molecules. The plate is washed to remove Gmd that has not been bound by the coated reference Gmd-binding antibody and to also remove the test, solution-phase Gmd-binding antibody as well as any complexes formed between the test, solution-phase Gmd-binding antibody and Gmd. The amount of bound Gmd is then measured using an appropriate Gmd detection reagent. A test Gmd-binding antibody in solution that is able to block binding of the coated reference Gmd-binding antibody to Gmd will be able to cause a decrease in the number of Gmd molecules that the coated reference Gmd-binding antibody can bind relative to the number of Gmd molecules that the coated reference Gmd-binding antibody can bind in the absence of the second, solution-phase test Gmd-binding antibody. The background signal for the assay is defined as the signal obtained in wells with the coated reference Gmd-binding antibody, solution-phase test Gmd-binding antibody, Gmd buffer only (i.e., no Gmd) and Gmd detection reagents. The positive control signal for the assay is defined as the signal obtained in wells with the coated reference Gmd-binding antibody, solution-phase test Gmd-binding antibody buffer only (i.e., no solution-phase test Gmd-binding antibody), Gmd detection reagents. The ELISA assay is run in such a manner so as to have the positive control signal at least 3 times the background signal. As a control for methodologic artifacts, the cross-blocking assay may be run in the format just described and also reversed, with the test Gmd-binding antibody as the coated antibody and the reference Gmd-binding antibody as the solution-phase antibody.

In some embodiments, the Gmd-binding antibody (e.g., a full length antibody or a Gmd-binding antibody fragment) specifically binds a Gmd fragment consisting of the amino acid sequence of SEQ ID NO:72 (e.g., a fragment consisting of amino acid residues 59-91 of SEQ ID NO:72) and has at least one characteristic selected from the group consisting of: (a) inhibits in vitro growth of S. aureus; (b) inhibits in vivo growth of S. aureus; (c) promotes clumping (clustering) of S. aureus (e.g., Xen29); (d) promotes cell-independent lysis of S. aureus; (e) decreases binary fission or inhibits cell division of S. aureus; (f) binds to a polypeptide consisting of the amino acid sequence of SEQ ID NO: 72 with a KD of ≤1 nM and ≥1 pM (e.g., as determined by BIACORE® analysis); and (g) binds to a polypeptide consisting of the amino acid sequence of SEQ ID NO:71 with a KD of ≤1 nM and ≥1 pM (e.g., as determined by BIACORE® analysis), but the Gmd-binding antibody does not have a VH sequence of SEQ ID NO:9, a VL sequence of SEQ ID NO:41, or a VH sequence of SEQ ID NO:9 and a VL sequence of SEQ ID NO:41. In some embodiments, the Gmd-binding antibody has 2, 3, 4, or 5 of the above characteristics. In some embodiments, the Gmd-binding antibody has at least 2, at least 3, at least 4, or at least 5, of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%. In further embodiments, the Gmd-binding antibody cross-blocks or competes for binding to Gmd with an antibody having a Gmd-binding VH and VL pair disclosed herein. In some embodiments, the Gmd-binding antibody is a full length antibody. In some embodiments, the Gmd-binding antibody is a Gmd-binding antibody fragment. In particular embodiments, the Gmd-binding antibody has at least 1, at least 2, at least 3, or at least 4, of the above characteristics, but does not comprise the VH sequence of SEQ ID NO:9 or the VL sequence of SEQ ID NO: 41.

In some embodiments, the Gmd-binding antibody specifically binds a Gmd fragment consisting of the amino acid sequence of SEQ ID NO:72 (e.g., a fragment consisting of amino acid residues 59-91 of SEQ ID NO:72) and inhibits the enzymatic activity of Gmd but the Gmd-binding antibody does not have a VH sequence of SEQ ID NO:9, a VL sequence of SEQ ID NO:41, or a VH sequence of SEQ ID NO:9 and a VL sequence of SEQ ID NO:41. In further embodiments, the Gmd-binding antibody inhibits the enzymatic activity of, a Gmd comprising the amino acid sequence of SEQ ID NO:81. In some embodiments, the Gmd-binding antibody inhibits the enzymatic activity of Gmd by at least 20%, at least 30%, at least 40% or at least 50%. In other embodiments, the Gmd-binding antibody inhibits the activity of Gmd by at least 60%, at least 70%, or at least 80%. In some embodiments, the Gmd-binding antibody inhibits the enzymatic activity of Gmd comprising the amino acid sequence of SEQ ID NO:81 by at least 20%, at least 30%, at least 40% or at least 50%. In other embodiments, the Gmd-binding antibody inhibits the enzymatic activity of Gmd comprising the amino acid sequence of SEQ ID NO:81 by at least 60%, at least 70%, or at least 80%. In some embodiments, the Gmd-binding antibody inhibits the enzymatic activity of S. aureus by 20% to 100%, 30% to 100%, 40% to 100%, 50% to 100%, 60% to 100%, 70% to 100%, or 80% to 100%. In further embodiments, the Gmd-binding antibody cross-blocks or competes for binding to Gmd with an antibody having a Gmd-binding VH and VL pair disclosed herein. In some embodiments, the Gmd-binding antibody is a full length antibody. In some embodiments, the Gmd-binding antibody is a Gmd-binding antibody fragment. In particular embodiments, the Gmd-binding antibody has at least 1, at least 2, at least 3, or at least 4, of the above characteristics, but does not comprise the VH sequence of SEQ ID NO:9 or the VL sequence of SEQ ID NO:41.

In some embodiments, the Gmd-binding antibody (e.g., a full length antibody or a Gmd-binding antibody fragment) specifically binds a Gmd fragment consisting of the amino acid sequence of SEQ ID NO:72 (e.g., a fragment consisting of amino acid residues 59-91 of SEQ ID NO:72) and inhibits in vitro growth of S. aureus, but the Gmd-binding antibody does not have a VH sequence of SEQ ID NO:9, a VL sequence of SEQ ID NO:41, or a VH sequence of SEQ ID NO:9 and a VL sequence of SEQ ID NO:41. In some embodiments, the Gmd-binding antibody inhibits in vitro growth of S. aureus by at least 20%, at least 30%, at least 40%, at least 50%, at least 60%, at least 70%, or at least 80%. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In some embodiments, the Gmd-binding antibody inhibits the in vivo growth of S. aureus by 20% to 100%, 30% to 100%, 40% to 100%, 50% to 100%, 60% to 100%, 70% to 100%, or 80% to 100%. In further embodiments, the Gmd-binding antibody cross-blocks or competes for binding to Gmd with an antibody having a Gmd-binding VH and VL pair disclosed herein. In some embodiments, the Gmd-binding antibody is a full length antibody. In some embodiments, the Gmd-binding antibody is a Gmd-binding antibody fragment. In particular embodiments, the Gmd-binding antibody has at least 1, at least 2, at least 3, or at least 4, of the above characteristics, but does not comprise the VH sequence of SEQ ID NO:9 or the VL sequence of SEQ ID NO:41.

In some embodiments, the Gmd-binding antibody (e.g., a full length antibody or a Gmd-binding antibody fragment) specifically binds a Gmd fragment consisting of the amino acid sequence of SEQ ID NO:72 (e.g., a fragment consisting of amino acid residues 59-91 of SEQ ID NO:72) and inhibits the cell independent lysis of S. aureus in vitro, but the Gmd-binding antibody does not have a VH sequence of SEQ ID NO:9, a VL sequence of SEQ ID NO:41, or a VH sequence of SEQ ID NO:9 and a VL sequence of SEQ ID NO:41. In some embodiments, the Gmd-binding antibody inhibits the cell independent lysis of S. aureus in vitro by at least 20%, at least 30%, at least 40%, at least 50%, at least 60%, at least 70%, or at least 80%. In some embodiments, the Gmd-binding antibody inhibits the cell independent lysis of S. aureus in vitro by 20% to 100%, 30% to 100%, 40% to 100%, 50% to 100%, 60% to 100%, 70% to 100%, or 80% to 100%. In some embodiments, the Gmd-binding antibody inhibits the cell independent lysis of S. aureus in vivo by 20% to 100%, 30% to 100%, 40% to 100%, 50% to 100%, 60% to 100%, 70% to 100%, or 80% to 100%. In further embodiments, the Gmd-binding antibody cross-blocks or competes for binding to Gmd with an antibody having a Gmd-binding VH and VL pair disclosed herein. In some embodiments, the Gmd-binding antibody is a full length antibody. In some embodiments, the Gmd-binding antibody is a Gmd-binding antibody fragment. In particular embodiments, the Gmd-binding antibody has at least 1, at least 2, at least 3, or at least 4, of the above characteristics, but does not comprise the VH sequence of SEQ ID NO:9 or the VL sequence of SEQ ID NO:41.

In some embodiments, the Gmd-binding antibody (e.g., a full length antibody or a Gmd-binding antibody fragment) specifically binds a Gmd fragment consisting of the amino acid sequence of SEQ ID NO:72 (e.g., a fragment consisting of amino acid residues 59-91 of SEQ ID NO:72) and inhibits cell division of S. aureus in vitro, but the Gmd-binding antibody does not have a VH sequence of SEQ ID NO:9, a VL sequence of SEQ ID NO:41, or a VH sequence of SEQ ID NO:9 and a VL sequence of SEQ ID NO:41. In some embodiments, the Gmd-binding antibody inhibits cell division of S. aureus in vitro by at least 20%, at least 30%, at least 40%, at least 50%, at least 60%, at least 70%, or at least 80%. In some embodiments, the Gmd-binding antibody inhibits cell division of S. aureus in vitro by 20% to 100%, 30% to 100%, 40% to 100%, 50% to 100%, 60% to 100%, 70% to 100%, or 80% to 100%. In further embodiments, the Gmd-binding antibody cross-blocks or competes for binding to Gmd with an antibody having a Gmd-binding VH and VL pair disclosed herein, but does not comprise the VH sequence of SEQ ID NO:9 or the VL sequence of SEQ ID NO:41.

In particular embodiments, the Gmd-binding antibody (e.g., a full length antibody or a Gmd-binding antibody fragment) specifically binds a Gmd fragment consisting of the amino acid sequence of SEQ ID NO:72 (e.g., a fragment consisting of amino acid residues 59-91 of SEQ ID NO: 72) inhibits S. aureus growth in vitro, but the Gmd-binding antibody does not have a VH sequence of SEQ ID NO:9, a VL sequence of SEQ ID NO:41, or a VH sequence of SEQ ID NO:9 and a VL sequence of SEQ ID NO:41 In another embodiment, a Gmd-binding antibody inhibits S. aureus growth in vitro by 5% to 100%, 10% to 95%, 10 to 90%, 10 to 85%, 10 to 80%, 10 to 75%, 10 to 70%, 10 to 75%, 10 to 70%, 10 to 60%, 10 to 55%, 10 to 50%, or 10 to 45%. In another embodiment, a Gmd-binding antibody inhibits S. aureus growth in vitro by at least 5%, 10%, 20%, 30%, 40%, 50%, 60%, 70%, 80%, 90%, or 95%, or by about 100%. In further embodiments, the Gmd-binding antibody cross-blocks or competes for binding to Gmd with an antibody having a Gmd-binding VH and VL pair disclosed herein, but does not comprise the VH sequence of SEQ ID NO:9 or the VL sequence of SEQ ID NO:41.

In particular embodiments, the Gmd-binding antibody (e.g., a full length antibody or a Gmd-binding antibody fragment) specifically binds a Gmd fragment consisting of the amino acid sequence of SEQ ID NO:72 (e.g., a fragment consisting of amino acid residues 59-91 of SEQ ID NO: 72) and inhibits S. aureus growth in vivo, but the Gmd-binding antibody does not have a VH sequence of SEQ ID NO:9, a VL sequence of SEQ ID NO:41, or a VH sequence of SEQ ID NO:9 and a VL sequence of SEQ ID NO:41. In another embodiment, a Gmd-binding antibody inhibits S. aureus growth in vivo by 5% to 100%, 10% to 95%, 10 to 90%, 10 to 85%, 10 to 80%, 10 to 75%, 10 to 70%, 10 to 75%, 10 to 70%, 10 to 60%, 10 to 55%, 10 to 50%, or 10 to 45%. In another embodiment, a Gmd-binding antibody inhibits S. aureus growth in vivo by at least 5%, 10%, 20%, 30%, 40%, 50%, 60%, 70%, 80%, 90%, or 95%, or by about 100%. In some embodiments, the Gmd-binding antibody inhibits or decreases one or more conditions associated with S. aureus infection. In a further embodiment, the Gmd-binding antibody treats a condition selected from fever, bacteremia, endocarditis, osteomyelitis, pneumonia, sepsis, dermonecrosis, and mastitis.

The inhibition of in vivo growth of Staphylococcus can be measured according to any number of suitable standards known in the art. In one embodiment, the in vivo growth of Staphylococcus is assessed according to a bio luminescence assay. By way of example, bio luminescent S. aureus (e.g., Xen 29; ATCC 12600) (Francis et al., Infect. Immun. 68(6):3594-600 (2000); see also Contag et al., Mol. Microbiol. 18(4):593-603 (1995), each of which is herein incorporated by reference in its entirety) is used to dose a transtibial implant with 500,000 CFU prior to surgical implant. Five week old female BALB/cJ mice can receive an intraperitoneal injection of saline or 1 mg of purified antibody/antibody fragment in 0.25 ml saline 3 days prior to surgery. The mice can be imaged to assess bioluminescence on various days (e.g., 0, 3, 5, 7, 11, and 14) and a comparison of BLI images can be compared to assess whether the Gmd-binding antibody inhibits in vivo growth of S. aureus relative to the saline control or a control mouse injected with a placebo antibody.

In another embodiment, the inhibition of in vivo growth of Staphylococcus is assessed according to bio film formation. By way of example, female Balb/c mice can be passively immunized intraperitoneally with antibody/antibody fragment or control at a dose of 40 mg/kg, and one day later each mouse can be infected with a trans-tibial stainless steel pin contaminated with a MRSA strain. On day 14 post-infection the pins can be removed and examined by scanning electron the tibia harvested. Using the resulting images, the lesion area can be measured in two different views (e.g., medial and lateral), which are added together and multiplied by the cortical thickness (see, Varrone et al., J. Orthop. Res. 32(10):1389-96 (2014), which is herein incorporated by reference in its entirety).

In another embodiment, in vivo growth of Staphylococcus is assessed by the presence (including frequency) or absence of Staphylococcus abscess communities (SACs) in the medullary canal or soft tissue surrounding the bone. By way of example, female Balb/c mice are passively immunized intraperitoneally with the Gmd-binding antibody or control at a dose of 40 mg/kg, and one day later each mouse can be infected with a trans-tibial stainless steel pin contaminated with a MRSA strain. After 14 days, the mice are euthanized and the tibia and associated soft tissue harvested. Histological samples are prepared and stained with Orange G/alcian blue (ABG/OH), and then the presence or absence of abscesses can be determined upon analysis of the histologic samples. (e.g., as described in the examples herein or otherwise in the art).

Pharmacodynamic parameters dependent on Gmd, or a microorganism that expresses Gmd such as S. aureus, can be measured as endpoints for in vivo testing of Gmd-binding antibodies (e.g., full length antibodies and Gmd-binding antibody fragments) in order to identify those Gmd-binding antibodies that are able to antagonize or neutralize Gmd or microorganisms expressing Gmd (e.g., S. aureus) and provide a therapeutic benefit. An antagonist Gmd-binding antibody is defined as one capable of causing a statistically significant change, as compared to vehicle-treated animals, in such a pharmacodynamic parameter. Such in vivo testing can be performed in any suitable mammal (e.g., mouse, rat, or monkey).

In some embodiments, the Gmd-binding antibody is a full-length antibody. In some embodiments, the Gmd-binding antibody is a monoclonal antibody, a recombinant antibody, a bi-specific antibody, or a multi-specific antibody.

In some embodiments, the Gmd-binding antibody is a Gmd-binding antibody fragment. In further embodiments, the Gmd-binding antibody fragment is a: Fab, Fab′, F(ab′)₂, Fv fragment, diabody, or single chain antibody molecule. In additional embodiments, the Gmd-antibody is a Fd, single chain Fv (scFv), disulfide linked Fv, V-NAR domain, IgNar, intrabody, IgGΔCH2, minibody, F(ab′)₃, tetrabody, triabody, diabody, single-domain antibody, DVD-Ig, Fcab, mAb², (scFv)₂, scFv-Fc or a bis-scFv.

In additional embodiments, the Gmd-binding antibody (e.g., a full length antibody or a Gmd-binding antibody fragment) is an antibody that includes a VH and a VL. In some embodiments, the Gmd-binding antibody further includes a heavy chain constant region or fragment thereof. In some embodiments, the Gmd-binding antibody comprises a heavy chain immunoglobulin constant region selected from the group consisting of: (a) a human IgA constant region, or fragment thereof; (b) a human IgD constant region, or fragment thereof; (c) a human IgE constant domain, or fragment thereof; (d) a human IgG1 constant region, or fragment thereof; (e) a human IgG2 constant region, or fragment thereof; (f) a human IgG3 constant region, or fragment thereof; (g) a human IgG4 constant region, or fragment thereof; and (h) a human IgM constant region, or fragment thereof. In certain embodiments, the Gmd-binding antibody comprises a heavy chain constant region or fragment thereof, e.g., a human IgG constant region or fragment thereof. In further embodiments, the Gmd-binding antibody comprises a heavy chain immunoglobulin constant domain that has, or has been mutated to have, altered effector function and/or half-life.

In particular embodiments, the Gmd-binding antibody (e.g., a full length antibody or a Gmd-binding antibody fragment) comprises an IgG1 heavy chain constant region containing a mutation that decreases effector function (see, e.g., Idusogie et al., J. Immunol. 166:2571-2575 (2001); Sazinsky et al., PNAS USA 105:20167-20172 (2008); Davis et al., J. Rheumatol. 34:2204-2210 (2007); Bolt et al., Eur. J. Immunol. 23:403-411 (1993); Alegre et al., Transplantation 57:1537-1543 (1994); Xu et al., Cell Immunol. 200:16-26 (2000); Cole et al., Transplantation 68:563-571 (1999); Hutchins et al., PNAS USA 92:11980-11984 (1995); Reddy et al., J. Immunol. 164:1925-1933 (2000); WO97/11971, and WO07/106585; U.S. Appl. Publ. 2007/0148167A1; McEarchern et al., Blood 109:1185-1192 (2007); Strohl, Curr. Op. Biotechnol. 20:685-691 (2009); and Kumagai et al., J. Clin. Pharmacol. 47:1489-1497 (2007), each of which is herein incorporated by reference in its entirety).

In some embodiments, the heavy chain constant region or fragment thereof of a Gmd-binding antibody includes one or more amino acid substitutions relative to a wild-type IgG constant domain wherein the modified IgG has decreased ADCC compared to the half-life of an IgG having the wild-type IgG constant domain. Examples of Fc sequence engineering that decrease ADCC include one or more modifications corresponding to: IgG1-K326W, E333S; IgG2-E333S; IgG1-N297A; IgG1-L234A, L235A; IgG2-V234A, G237A; IgG4-L235A, G237A, E318A; IgG4-S228P, L236E; IgG2-EU sequence 118-260; IgG4-EU sequence 261-447; IgG2-H268Q, V309L, A330S, A331S; IgG1-C220S, C226S, C229S, P238S; IgG1-C226S, C229S, E233P, L234V, L235A; and IgG1-L234F, L235E, P331S, wherein the position numbering is according to the EU index as in Kabat.

In certain embodiments, a Gmd-binding antibody comprises a heavy chain immunoglobulin constant domain that has, or has been mutated to have, reduced CDC activity. In particular embodiments, the Gmd-binding antibody comprises an IgG1 heavy chain constant region containing a mutation that decreases CDC activity (see, e.g., WO97/11971 and WO07/106585; U.S. Appl. Publ. 2007/0148167A1; McEarchern et al., Blood 109:1185-1192 (2007); Hayden-Ledbetter et al., Clin. Cancer 15:2739-2746 (2009); Lazar et al., PNAS USA 103:4005-4010 (2006); Bruckheimer et al., Neoplasia 11:509-517 (2009); Strohl, Curr. Op. Biotechnol. 20:685-691 (2009); and Sazinsky et al., PNAS USA 105:20167-20172 (2008); each of which is herein incorporated by reference in its entirety). Examples of Fc sequence engineering modifications contained in a Gmd-binding antibody that decrease CDC include one or more modifications corresponding to: IgG1-S239D, A330L, 1332E; IgG2 EU sequence 118-260; IgG4-EU sequence 261-447; IgG2-H268Q, V309L, A330S, A331S; IgG1-C226S, C229S, E233P, L234V, L235A; IgG1-L234F, L235E, P331S; and IgG1-C226S, P230S.

In further embodiments, the heavy chain constant region or fragment thereof includes one or more amino acid substitutions relative to a wild-type IgG constant domain wherein the modified IgG has an increased half-life compared to the half-life of an IgG having the wild-type IgG constant domain. For example, the IgG constant domain can contain one or more amino acid substitutions of amino acid residues at positions 251-257, 285-290, 308-314, 385-389, and 428-436, wherein the amino acid position numbering is according to the EU index as set forth in Kabat. In certain embodiments, the IgG constant domain contains one or more of: a substitution of the amino acid at Kabat position 252 with Tyr, Phe, Trp, or Thr; a substitution of the amino acid at Kabat position 254 with Thr; a substitution of the amino acid at Kabat position 256 with Ser, Arg, Gln, Glu, Asp, or Thr; a substitution of the amino acid at Kabat position 257 with Leu; a substitution of the amino acid at Kabat position 309 with Pro; a substitution of the amino acid at Kabat position 311 with Ser; a substitution of the amino acid at Kabat position 428 with Thr, Leu, Phe, or Ser; a substitution of the amino acid at Kabat position 433 with Arg, Ser, Iso, Pro, or Gln; or a substitution of the amino acid at Kabat position 434 with Trp, Met, Ser, His, Phe, or Tyr. In a particular embodiment, the IgG constant domain can contain amino acid substitutions relative to a wild-type human IgG constant domain including a substitution of the amino acid at Kabat position 252 with Tyr, a substitution of the amino acid at Kabat position 254 with Thr, and a substitution of the amino acid at Kabat position 256 with Glu.

In additional embodiments, the Gmd-binding antibody (e.g., a full length antibody or a Gmd-binding antibody fragment) comprises a light chain immunoglobulin constant region. In a further embodiment, the Gmd-binding antibody comprises a human Ig kappa constant region or a human Ig lambda constant region.

In some embodiments, the Gmd-binding antibody (e.g., a full length antibody or a Gmd-binding antibody fragment) comprises a set of CDRs: VH-CDR1, VH-CDR2, VH-CDR3, VL-CDR1, VL-CDR2 and VL-CDR3, wherein the CDRs are present in a VH and a VL pair disclosed in Table 1, but wherein the Gmd-binding antibody does not comprise the VH sequence of SEQ ID NO:9, the VL sequence of SEQ ID NO:41, or the VH sequence of SEQ ID NO:9 and the VL sequence of SEQ ID NO: 41.

In some embodiments, the Gmd-binding antibody (e.g., a full length antibody or a Gmd-binding antibody fragment) specifically binds a Gmd fragment consisting of the amino acid sequence of SEQ ID NO:72 (e.g., a fragment consisting of amino acid residues 59-91 of SEQ ID NO:72) and comprises a set of CDRs: (a) VH-CDR1, VH-CDR2, and VH-CDR3, or (b) VL-CDR1, VL-CDR2, and VL-CDR3, wherein the set of CDRs is has a total of one, two, three, four, five, six, seven, eight, nine, ten, or fewer than ten, amino acid substitutions, deletions, and/or insertions from a reference set of CDRs disclosed in Table 1. In some embodiments, the Gmd-binding antibody comprises a set of CDRs: (a) VH-CDR1, VH-CDR2, and VH-CDR3, or (b) VL-CDR1, VL-CDR2, and VL-CDR3, wherein at least one, two, or all three of the CDRs does not have the same sequence as the corresponding CDR in the amino acid sequence of a VH of SEQ ID NO:9, or a VL of SEQ ID NO:41.

In some embodiments, the Gmd-binding antibody (e.g., a full length antibody or a Gmd-binding antibody fragment) antibody (e.g., a full length antibody or a Gmd-binding antibody fragment) specifically binds a Gmd fragment consisting of the amino acid sequence of SEQ ID NO:72 (e.g., a fragment consisting of amino acid residues 59-91 of SEQ ID NO:72) and comprises a set of CDRs: VH-CDR1, VH-CDR2, VH-CDR3, VL-CDR1, VL-CDR2, and VL-CDR3, wherein the set of CDRs has a total of one, two, three, four, five, six, seven, eight, nine, ten, or fewer than ten, amino acid substitutions, deletions, and/or insertions from a reference from a reference set of CDRs in which: (a) (i) VH-CDR1 comprises the amino acid sequence of SEQ ID NO:10; (ii) VH-CDR2 comprises the amino acid sequence of SEQ ID NO:11; (iii) VH-CDR3 comprises the amino acid sequence of SEQ ID NO: 12; (iv) VL-CDR1 comprises the amino acid sequence of SEQ ID NO:42; (v) VL-CDR2 comprises the amino acid sequence of SEQ ID NO:43; and (vi) VL-CDR3 comprises the amino acid sequence of SEQ ID NO:44. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%. In some embodiments, the Gmd-binding antibody has 2, 3, or 4 of the above characteristics. In some embodiments, the Gmd-binding antibody has at least 2, at least 3, or at least 4, of the above characteristics.

In some embodiments, the Gmd-binding antibody (e.g., a full length antibody or a Gmd-binding antibody fragment) comprises a VH comprising a VH-CDR1 of SEQ ID NO:10, a VH-CDR2 of SEQ ID NO:11, and a VH-CDR3 of SEQ ID NO: 12, and VL comprising a VH-CDR1 of SEQ ID NO: 42, a VH-CDR2 of SEQ ID NO:43, and a VH-CDR3 of SEQ ID NO:44, and further comprises a framework sequence selected from the group consisting of (a) a VH framework 1 sequence of SEQ ID NO: 13 or 14; (b) a VH framework 2 sequence of SEQ ID NO:16-18, or 19; (c) a VH framework 3 sequence of SEQ ID NO:21; (d) a VL framework 1 sequence of SEQ ID NO:45, 46, or 47; (e) a VL framework 2 sequence of SEQ ID NO:49, 50, or 51; and (f) a VL framework 3 sequence of SEQ ID NO: 53-58, or 59. In some embodiments, the Gmd-binding antibody comprises a VH framework 3 sequence of SEQ ID NO:21. In some embodiments, the Gmd-binding antibody comprises a VH framework 3 sequence of SEQ ID NO:21. In some embodiments, the Gmd-binding antibody comprises a VH of SEQ ID NO:1-7, or 8. In further embodiments, the Gmd-binding antibody comprises a VL framework 4 sequence of SEQ ID NO:61.

In some embodiments, the Gmd-binding antibody (e.g., a full length antibody or a Gmd-binding antibody fragment) comprises a VH comprising a VH-CDR1 of SEQ ID NO: 10, a VH-CDR2 of SEQ ID NO: 11, and a VH-CDR3 of SEQ ID NO: 12, and VL comprising a VL-CDR1 of SEQ ID NO: 42, a VL-CDR2 of SEQ ID NO:43, and a VL-CDR3 of SEQ ID NO:44, and comprises a VH framework sequence selected from the group consisting of (a) a framework 1 sequence of SEQ ID NO: 13 and a framework 2 sequence of SEQ ID NO: 16; (b) a framework 1 sequence of SEQ ID NO: 13 and a framework 2 sequence of SEQ ID NO: 17; (c) a framework 1 sequence of SEQ ID NO:13 and a framework 2 sequence of SEQ ID NO:18; (d) a framework 1 sequence of SEQ ID NO:13 and a framework 2 sequence of SEQ ID NO:19; (e) a framework 1 sequence of SEQ ID NO:14 and a framework 2 sequence of SEQ ID NO:16; (f) a framework 1 sequence of SEQ ID NO:14 and a framework 2 sequence of SEQ ID NO: 17; and (g) a framework 1 sequence of SEQ ID NO: 14 and a framework 2 sequence of SEQ ID NO:18. In further embodiments, the Gmd-binding antibody comprises a VH framework 4 sequence of SEQ ID NO:24. In some embodiments, the Gmd-binding antibody comprises a VH of SEQ ID NO: 1-7, or 8. In further embodiments, the Gmd-binding antibody comprises a VL framework 4 sequence of SEQ ID NO:61. In some embodiments, the Gmd-binding antibody comprises a VH framework 3 sequence of SEQ ID NO:21. In some embodiments, the Gmd-binding antibody comprises a VH of SEQ ID NO:1-7, or 8.

In some embodiments, the Gmd-binding antibody (e.g., a full length antibody or a Gmd-binding antibody fragment) comprises a VH comprising a VH-CDR1 of SEQ ID NO:10, a VH-CDR2 of SEQ ID NO:11, and a VH-CDR3 of SEQ ID NO:12, and VL comprising a VH-CDR1 of SEQ ID NO: 42, a VH-CDR2 of SEQ ID NO:43, and a VH-CDR3 of SEQ ID NO:44, and comprises a VH framework 3 sequence of SEQ ID NO:21. In further embodiments, the Gmd-binding antibody comprises a VH framework 4 sequence of SEQ ID NO:24. In some embodiments, the Gmd-binding antibody comprises a VH of SEQ ID NO:1-7, or 8.

In some embodiments, the Gmd-binding antibody (e.g., a full length antibody or a Gmd-binding antibody fragment) comprises a VH comprising a VH-CDR1 of SEQ ID NO:10, a VH-CDR2 of SEQ ID NO:11, and a VH-CDR3 of SEQ ID NO:12, and VL comprising a VH-CDR1 of SEQ ID NO: 42, a VH-CDR2 of SEQ ID NO:43, and a VH-CDR3 of SEQ ID NO:44, and comprises a VL framework sequence selected from the group consisting of: (a) a framework 1 sequence of SEQ ID NO: 45 and a framework 2 sequence of SEQ ID NO:49; (b) a framework 1 sequence of SEQ ID NO:46 and a framework 2 sequence of SEQ ID NO:50; (c) a framework 1 sequence of SEQ ID NO:47 and a framework 2 sequence of SEQ ID NO:50; (d) a framework 1 sequence of SEQ ID NO:47 and a framework 2 sequence of SEQ ID NO:51; (e) a framework 2 sequence of SEQ ID NO:49 and a framework 3 sequence of SEQ ID NO:53; (f) a framework 2 sequence of SEQ ID NO:49 and a framework 3 sequence of SEQ ID NO:55; (g) a framework 2 sequence of SEQ ID NO:50 and a framework 3 sequence of SEQ ID NO:54; (h) a framework 2 sequence of SEQ ID NO:50 and a framework 3 sequence of SEQ ID NO:55; (i) a framework 2 sequence of SEQ ID NO:50 and a framework 3 sequence of SEQ ID NO:56; (j) a framework 2 sequence of SEQ ID NO:50 and a framework 3 sequence of SEQ ID NO:57; and (k) a framework 2 sequence of SEQ ID NO:51 and a framework 3 sequence of SEQ ID NO:58. In some embodiments, the Gmd-binding antibody comprises a VH framework 3 sequence of SEQ ID NO:21. In some embodiments, the Gmd-binding antibody comprises a VH of SEQ ID NO:1-7, or 8.

In some embodiments, a Gmd-binding antibody (e.g., a full length antibody or a Gmd-binding antibody fragment) comprises a VH or a VL which has at least 90%, 95%, 96%, 97%, 98%, 99%, or 100% sequence identity to a reference VH or VL disclosed in Table 1, and wherein the sequence of the VH is not SEQ ID NO:9 or the sequence of the VL is not SEQ ID NO:41, respectively. In some embodiments, the Gmd-binding antibody comprises a VH and VL which has at least 90%, 95%, 96%, 97%, 98%, 99%, or 100% sequence identity to a reference VH and VL disclosed in Table 1, and wherein the sequence of the VH is not SEQ ID NO:9 and/or the sequence of the VL is not SEQ ID NO: 41. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody has at least 2, at least 3, or at least 4, of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%-In some embodiments, the Gmd-binding antibody inhibits in vivo growth of S. aureus by 20% to 100%, 30% to 100%, 40% to 100%, 50% to 100%, 60% to 100%, 70% to 100%, or 80% to 100%.

In some embodiments, the Gmd-binding antibody (e.g., a full length antibody or a Gmd-binding antibody fragment) comprises a VH having: (a) at least 90%, 95%, 96%, 97%, 98%, 99%, or 100% sequence identity to SEQ ID NO:1; (b) at least 90%, 95%, 96%, 97%, 98%, 99%, or 100% sequence identity to SEQ ID NO:2; (c) at least 90%, 95%, 96%, 97%, 98%, 99%, or 100% sequence identity to SEQ ID NO:3; and (d) a VH having at least 90%, 95%, 96%, 97%, 98%, 99%, or 100% sequence identity to SEQ ID NO:4; (e) at least 90%, 95%, 96%, 97%, 98%, 99%, or 100% sequence identity to SEQ ID NO:5; (f) at least 90%, 95%, 96%, 97%, 98%, 99%, or 100% sequence identity to SEQ ID NO:6; (g) at least 90%, 95%, 96%, 97%, 98%, 99%, or 100% sequence identity to SEQ ID NO: 7; and (h) a VH having at least 90%, 95%, 96%, 97%, 98%, 99%, or 100% sequence identity to SEQ ID NO:8; and does not comprise the VH sequence of SEQ ID NO:9. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody has at least 2, at least 3, or at least 4, of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%.

In some embodiments, the Gmd-binding antibody (e.g., a full length antibody or a Gmd-binding antibody fragment) comprises a VL having: (a) at least 90%, 95%, 96%, 97%, 98%, 99%, or 100% sequence identity to SEQ ID NO:33; (b) at least 90%, 95%, 96%, 97%, 98%, 99%, or 100% sequence identity to SEQ ID NO:34; (c) at least 90%, 95%, 96%, 97%, 98%, 99%, or 100% sequence identity to SEQ ID NO:35; and (d) a VH having at least 90%, 95%, 96%, 97%, 98%, 99%, or 100% sequence identity to SEQ ID NO:36; (e) at least 90%, 95%, 96%, 97%, 98%, 99%, or 100% sequence identity to SEQ ID NO:37; (f) at least 90%, 95%, 96%, 97%, 98%, 99%, or 100% sequence identity to SEQ ID NO:38; (g) at least 90%, 95%, 96%, 97%, 98%, 99%, or 100% sequence identity to SEQ ID NO: 39; and (h) a VH having at least 90%, 95%, 96%, 97%, 98%, 99%, or 100% sequence identity to SEQ ID NO:40; and does not comprise the VL sequence of SEQ ID NO:41. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody has at least 2, at least 3, or at least 4, of the above characteristics. In further embodiments, the Gmd-binding antibody inhibits in vitro growth of S. aureus by 20% to 100%, 30% to 100%, 40% to 100%, 50% to 100%, 60% to 100%, 70% to 100%, or 80% to 100%.

In one embodiment, the Gmd-binding antibody (e.g., a full length antibody or a Gmd-binding antibody fragment) comprises a VH having at least 90%, 95%, 96%, 97%, 98%, 99%, or 100% sequence identity to SEQ ID NO:1-7 or 8, and a VL having at least 90%, 95%, 96%, 97%, 98%, 99%, or 100% sequence identity to SEQ ID NO:33-39, or 40; and wherein the Gmd-binding antibody does not contain a VH having the sequence of SEQ ID NO:9 and a VL having the sequence of SEQ ID NO: 41. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO: 72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody has at least 2, at least 3, or at least 4, of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In some embodiments, the Gmd-binding antibody has at least 2, at least 3, or at least 4, of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%.

In some embodiments, the Gmd-binding antibody (e.g., a full length antibody or a Gmd-binding antibody fragment) comprises a VH and a VL pair selected from the group consisting of: (a) (i) a VH having at least 90%, 95%, 96%, 97%, 98%, 99%, or 100% sequence identity to SEQ ID NO:1, and a VL having at least 90%, 95%, 96%, 97%, 98%, 99%, or 100% sequence identity to SEQ ID NO: 34 or 35; (b) a VH having at least 90%, 95%, 96%, 97%, 98%, 99%, or 100% sequence identity to SEQ ID NO:2, and a VL having at least 90%, 95%, 96%, 97%, 98%, 99%, or 100% sequence identity to SEQ ID NO:34, 35, or 37; (c) a VH having at least 90%, 95%, 96%, 97%, 98%, 99%, or 100% sequence identity to SEQ ID NO:3 and a VL having at least 90%, 95%, 96%, 97%, 98%, 99%, or 100% sequence identity to SEQ ID NO:33, 35, 36, 38, or 39; (d) a VH having at least 90%, 95%, 96%, 97%, 98%, 99%, or 100% sequence identity to SEQ ID NO:4, and a VL having at least 90%, 95%, 96%, 97%, 98%, 99%, or 100% sequence identity to SEQ ID NO:39; (e) a VH having at least 90%, 95%, 96%, 97%, 98%, 99%, or 100% sequence identity to SEQ ID NO:5, and a VL having at least 90%, 95%, 96%, 97%, 98%, 99%, or 100% sequence identity to SEQ ID NO:35, 36, 38, or 39; (f) a VH having at least 90%, 95%, 96%, 97%, 98%, 99%, or 100% sequence identity to SEQ ID NO: 6, and a VL having at least 90%, 95%, 96%, 97%, 98%, 99%, or 100% sequence identity to SEQ ID NO: 35, 36, or 38; (g) a VH having at least 90%, 95%, 96%, 97%, 98%, 99%, or 100% sequence identity to SEQ ID NO:7, and a VL having at least 90%, 95%, 96%, 97%, 98%, 99%, or 100% sequence identity to SEQ ID NO:34 or 35; (h) a VH having at least 90%, 95%, 96%, 97%, 98%, 99%, or 100% sequence identity to SEQ ID NO:8, and a VL having at least 90%, 95%, 96%, 97%, 98%, 99%, or 100% sequence identity to SEQ ID NO:33-38, or 40; but does not comprise the VH sequence of SEQ ID NO:9, the VL sequence of SEQ ID NO:41, or the VH sequence of SEQ ID NO:9 and the VL sequence of SEQ ID NO:41.

In some embodiments, the Gmd-binding antibody (e.g., a full length antibody or a Gmd-binding antibody fragment) comprises a VH and a VL pair selected from the group consisting of: (a) a VH having at least 90%, 95%, 96%, 97%, 98%, 99%, or 100% sequence identity to SEQ ID NO:1, and a VL having at least 90%, 95%, 96%, 97%, 98%, 99%, or 100% sequence identity to SEQ ID NO: 34; (b) a VH having at least 90%, 95%, 96%, 97%, 98%, 99%, or 100% sequence identity to SEQ ID NO: 1, and a VL having at least 90%, 95%, 96%, 97%, 98%, 99%, or 100% sequence identity to SEQ ID NO:35; (c) a VH having at least 90%, 95%, 96%, 97%, 98%, 99%, or 100% sequence identity to SEQ ID NO:2, and a VL having at least 90%, 95%, 96%, 97%, 98%, 99%, or 100% sequence identity to SEQ ID NO:34; (d) a VH having at least 90%, 95%, 96%, 97%, 98%, 99%, or 100% sequence identity to SEQ ID NO:2, and a VL having at least 90%, 95%, 96%, 97%, 98%, 99%, or 100% sequence identity to SEQ ID NO:35; (e) a VH having at least 90%, 95%, 96%, 97%, 98%, 99%, or 100% sequence identity to SEQ ID NO:2, and a VL having at least 90%, 95%, 96%, 97%, 98%, 99%, or 100% sequence identity to SEQ ID NO:37; (f) a VH having at least 90%, 95%, 96%, 97%, 98%, 99%, or 100% sequence identity to SEQ ID NO:3, and a VL having at least 90%, 95%, 96%, 97%, 98%, 99%, or 100% sequence identity to SEQ ID NO:33; (g) a VH having at least 90%, 95%, 96%, 97%, 98%, 99%, or 100% sequence identity to SEQ ID NO:3, and a VL having at least 90%, 95%, 96%, 97%, 98%, 99%, or 100% sequence identity to SEQ ID NO:35; (h) a VH having at least 90%, 95%, 96%, 97%, 98%, 99%, or 100% sequence identity to SEQ ID NO:3, and a VL having at least 90%, 95%, 96%, 97%, 98%, 99%, or 100% sequence identity to SEQ ID NO:36; (i) a VH having at least 90%, 95%, 96%, 97%, 98%, 99%, or 100% sequence identity to SEQ ID NO:3, and a VL having at least 90%, 95%, 96%, 97%, 98%, 99%, or 100% sequence identity to SEQ ID NO:38; (j) a VH having at least 90%, 95%, 96%, 97%, 98%, 99%, or 100% sequence identity to SEQ ID NO:3, and a VL having at least 90%, 95%, 96%, 97%, 98%, 99%, or 100% sequence identity to SEQ ID NO: 39; (k) a VH having at least 90%, 95%, 96%, 97%, 98%, 99%, or 100% sequence identity to SEQ ID NO: 4, and a VL having at least 90%, 95%, 96%, 97%, 98%, 99%, or 100% sequence identity to SEQ ID NO:39; (l) a VH having at least 90%, 95%, 96%, 97%, 98%, 99%, or 100% sequence identity to SEQ ID NO:5, and a VL having at least 90%, 95%, 96%, 97%, 98%, 99%, or 100% sequence identity to SEQ ID NO:35; (m) a VH having at least 90%, 95%, 96%, 97%, 98%, 99%, or 100% sequence identity to SEQ ID NO:5, and a VL having at least 90%, 95%, 96%, 97%, 98%, 99%, or 100% sequence identity to SEQ ID NO:36; (n) a VH having at least 90%, 95%, 96%, 97%, 98%, 99%, or 100% sequence identity to SEQ ID NO:5, and a VL having at least 90%, 95%, 96%, 97%, 98%, 99%, or 100% sequence identity to SEQ ID NO:38; (o) a VH having at least 90%, 95%, 96%, 97%, 98%, 99%, or 100% sequence identity to SEQ ID NO:5, and a VL having at least 90%, 95%, 96%, 97%, 98%, 99%, or 100% sequence identity to SEQ ID NO:39; (p) a VH having at least 90%, 95%, 96%, 97%, 98%, 99%, or 100% sequence identity to SEQ ID NO:6, and a VL having at least 90%, 95%, 96%, 97%, 98%, 99%, or 100% sequence identity to SEQ ID NO:35; (q) a VH having at least 90%, 95%, 96%, 97%, 98%, 99%, or 100% sequence identity to SEQ ID NO:6, and a VL having at least 90%, 95%, 96%, 97%, 98%, 99%, or 100% sequence identity to SEQ ID NO:36; (r) a VH having at least 90%, 95%, 96%, 97%, 98%, 99%, or 100% sequence identity to SEQ ID NO:6, and a VL having at least 90%, 95%, 96%, 97%, 98%, 99%, or 100% sequence identity to SEQ ID NO:38; (s) a VH having at least 90%, 95%, 96%, 97%, 98%, 99%, or 100% sequence identity to SEQ ID NO:7, and a VL having at least 90%, 95%, 96%, 97%, 98%, 99%, or 100% sequence identity to SEQ ID NO: 34; (t) a VH having at least 90%, 95%, 96%, 97%, 98%, 99%, or 100% sequence identity to SEQ ID NO: 7, and a VL having at least 90%, 95%, 96%, 97%, 98%, 99%, or 100% sequence identity to SEQ ID NO:35; (u) a VH having at least 90%, 95%, 96%, 97%, 98%, 99%, or 100% sequence identity to SEQ ID NO:8, and a VL having at least 90%, 95%, 96%, 97%, 98%, 99%, or 100% sequence identity to SEQ ID NO:33; (v) a VH having at least 90%, 95%, 96%, 97%, 98%, 99%, or 100% sequence identity to SEQ ID NO:8, and a VL having at least 90%, 95%, 96%, 97%, 98%, 99%, or 100% sequence identity to SEQ ID NO:34; (w) a VH having at least 90%, 95%, 96%, 97%, 98%, 99%, or 100% sequence identity to SEQ ID NO:8, and a VL having at least 90%, 95%, 96%, 97%, 98%, 99%, or 100% sequence identity to SEQ ID NO:35; (x) a VH having at least 90%, 95%, 96%, 97%, 98%, 99%, or 100% sequence identity to SEQ ID NO:8, and a VL having at least 90%, 95%, 96%, 97%, 98%, 99%, or 100% sequence identity to SEQ ID NO:36; (y) a VH having at least 90%, 95%, 96%, 97%, 98%, 99%, or 100% sequence identity to SEQ ID NO:8, and a VL having at least 90%, 95%, 96%, 97%, 98%, 99%, or 100% sequence identity to SEQ ID NO:37; (z) a VH having at least 90%, 95%, 96%, 97%, 98%, 99%, or 100% sequence identity to SEQ ID NO:8, and a VL having at least 90%, 95%, 96%, 97%, 98%, 99%, or 100% sequence identity to SEQ ID NO:38; and (aa) a VH having at least 90%, 95%, 96%, 97%, 98%, 99%, or 100% sequence identity to SEQ ID NO:8, and a VL having at least 90%, 95%, 96%, 97%, 98%, 99%, or 100% sequence identity to SEQ ID NO: 40; and wherein the Gmd-binding antibody does not comprise the VH sequence of SEQ ID NO:9, the VL sequence of SEQ ID NO:41, or the VH sequence of SEQ ID NO:9 and the VL sequence of SEQ ID NO:41. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody has at least 2, at least 3, or at least 4, of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%.

In some embodiments, the VH and/or VL amino acid sequence(s) that specifically binds a Gmd fragment consisting of the amino acid sequence of SEQ ID NO:72 (e.g., a fragment consisting of amino acid residues 59-91 of SEQ ID NO:72) comprises 8, 7, 6, 5, 4, 3, 2, 1 amino acid additions, substitutions (e.g., conservative substitutions) or deletions relative to a VH or a VL sequence set forth in Table 1, but does not comprise the VH sequence of SEQ ID NO:9, the VL sequence of SEQ ID NO: 41, or the VH sequence of SEQ ID NO:9 and the VL sequence of SEQ ID NO:41. In additional embodiments, the VH and VL amino acid sequence that specifically binds a Gmd fragment consisting of the amino acid sequence of SEQ ID NO:72 (e.g., a fragment consisting of amino acid residues 59-91 of SEQ ID NO:72) and comprises 1, 2, 3, 4, 5 or more amino acid additions, substitutions (e.g., conservative substitutions) or deletions relative to a VH and VL sequence set forth in Table 1, but do not comprise the VH sequence of SEQ ID NO:9, the VL sequence of SEQ ID NO:41, or the VH sequence of SEQ ID NO:9 and the VL sequence of SEQ ID NO:41. Gmd-binding antibody containing VH and VL regions having one or more substitutions, deletions and/or insertions (e.g., conservative substitutions) compared to a reference VH region or VL region can routinely be obtained by mutagenesis (e.g., site-directed or PCR-mediated mutagenesis) of nucleic acid molecules encoding VH and/or VL regions, followed by testing of the encoded altered antibody for binding to Gmd and optionally testing for retained function using the functional assays described herein or otherwise known in the art.

In some embodiments, a Gmd-binding antibody (e.g., a full length antibody or a Gmd-binding antibody fragment) comprises a VH or a VL which has a total of 1, 2, 3, 4, 5, 6, 7, 8, 9, 10, fewer than 15, or zero, amino acid substitutions, deletions, and/or insertions compared to a reference VH or VL disclosed in Table 1, but do not comprise the VH sequence of SEQ ID NO:9, the VL sequence of SEQ ID NO:41, or the VH sequence of SEQ ID NO:9 and the VL sequence of SEQ ID NO: 41. In further embodiments, the Gmd-binding antibody comprises a VH and VL pair which has a total of 1, 2, 3, 4, 5, 6, 7, 8, 9, 10, fewer than 15, or zero, amino acid substitutions, deletions, and/or insertions compared to a reference VH and VL pair disclosed in Table 1, but do not comprise the VH sequence of SEQ ID NO:9, the VL sequence of SEQ ID NO:41, or the VH sequence of SEQ ID NO:9 and the VL sequence of SEQ ID NO:41.

In some embodiments, the Gmd-binding antibody (e.g., a full length antibody or a Gmd-binding antibody fragment) specifically binds a Gmd fragment consisting of the amino acid sequence of SEQ ID NO:72 (e.g., a fragment consisting of amino acid residues 59-91 of SEQ ID NO:72) and comprises a VH and a VL pair selected from the group consisting of: (a) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:1, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:33-39, or 40; (b) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:2, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; (c) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:3, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; (d) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:4, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; (e) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:5, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; (f) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:6, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; (g) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:7, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; and (h) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:8, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-39, or 40; and wherein the Gmd-binding antibody does not comprise the VH sequence of SEQ ID NO:9, the VL sequence of SEQ ID NO:41, or the VH sequence of SEQ ID NO:9 and the VL sequence of SEQ ID NO:41.

In some embodiments, the Gmd-binding antibody (e.g., a full length antibody or a Gmd-binding antibody fragment) comprises a VH sequence having a total of 1, 2, 3, 4, 5, 6, 7, 8, 9, 10, fewer than 15, or zero, amino acid substitutions, deletions, and/or insertions from a reference VH sequence selected from the group consisting of (a) SEQ ID NO:1, (b) SEQ ID NO:2, (c) SEQ ID NO:3, (d) SEQ ID NO:4, (e) SEQ ID NO:5, (f) SEQ ID NO:6, (g) SEQ ID NO:7, and (h) SEQ ID NO:8, but does not have the sequence of SEQ ID NO:9. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody has at least 2, at least 3, or at least 4, of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%.

In some embodiments, the Gmd-binding antibody (e.g., a full length antibody or a Gmd-binding antibody fragment) comprises a VL sequence having a total of 1, 2, 3, 4, 5, 6, 7, 8, 9, 10, fewer than 15, or zero, amino acid substitutions, deletions, and/or insertions from a reference VL sequence selected from the group consisting of (a) SEQ ID NO:33, (b) SEQ ID NO:34, (c) SEQ ID NO:35, (d) SEQ ID NO:36, (e) SEQ ID NO:37, (f) SEQ ID NO:38, (g) SEQ ID NO:39, and (h) SEQ ID NO:40, but does not have the sequence of SEQ ID NO:41. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody has at least 2, at least 3, or at least 4, of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%.

In some embodiments, the Gmd-binding antibody (e.g., a full length antibody or a Gmd-binding antibody fragment) comprises a VH and a VL pair selected from the group consisting of: (a) (i) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:1, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:34 or 35; (b) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:2, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:34, 35, or 37; (c) a VH having sequence having a total of 1, 2, 3, 4, 5, 6, 7, 8, 9, 10, fewer than 15, or zero, amino acid substitutions, deletions, and/or insertions from a reference VH sequence of SEQ ID NO:3, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:33, 35, 36, 38, or 39; (d) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:4, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:39; (e) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:5, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:35, 36, 38, or 39; (f) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:6, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:35, 36, or 38; (g) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:7, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:34 or 35; and (h) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:8, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO:33-38 or 40; and wherein the Gmd-binding antibody does not comprise the VH sequence of SEQ ID NO:9, the VL sequence of SEQ ID NO:41, or the VH sequence of SEQ ID NO:9 and the VL sequence of SEQ ID NO: 41. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody has at least 2, at least 3, or at least 4, of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%.

In a further embodiment, the Gmd-binding antibody (e.g., a full length antibody or a Gmd-binding antibody fragment) comprises a VH and a VL pair wherein the VH sequence has a total of 1, 2, 3, 4, 5, 6, 7, 8, 9, 10, fewer than 15, or zero, amino acid substitutions, deletions, and/or insertions from a reference VH sequence of SEQ ID NO:4; and the VL sequence has a total of 1, 2, 3, 4, 5, 6, 7, 8, 9, 10, fewer than 15, or zero, amino acid substitutions, deletions, and/or insertions from a reference VL sequence of SEQ ID NO:13. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%. In some embodiments, the Gmd-binding antibody has 2, 3, or 4 of the above characteristics. In some embodiments, the Gmd-binding antibody has at least 2, at least 3, or at least 4, of the above characteristics.

In a further embodiment, the Gmd-binding antibody (e.g., a full length antibody or a Gmd-binding antibody fragment) comprises a VH and a VL pair wherein the VH sequence has a total of 1, 2, 3, 4, 5, 6, 7, 8, 9, 10, fewer than 15, or zero, amino acid substitutions, deletions, and/or insertions from a reference VH sequence of SEQ ID NO:9; and the VL sequence has a total of 1, 2, 3, 4, 5, 6, 7, 8, 9, 10, fewer than 15, or zero, amino acid substitutions, deletions, and/or insertions from a reference VL sequence of SEQ ID NO:41. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%. In some embodiments, the Gmd-binding antibody has 2, 3, or 4 of the above characteristics. In some embodiments, the Gmd-binding antibody has at least 2, at least 3, or at least 4, of the above characteristics.

In some embodiments, the Gmd-binding antibody (e.g., a full length antibody or a Gmd-binding antibody fragment) comprises a VH sequence of SEQ ID NO:8 and a VL sequence of SEQ ID NO: 40. In some embodiments, a Gmd-binding antibody cross-blocks or competes for binding to Gmd with an antibody comprising a VH sequence of SEQ ID NO:8 and a VL sequence of SEQ ID NO: 40. In further embodiments, the Gmd-binding antibody binds the same epitope of Gmd as an antibody comprising a VH sequence of SEQ ID NO:8 and a VL sequence of SEQ ID NO:40. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%. In some embodiments, the Gmd-binding antibody has 2, 3, or 4 of the above characteristics. In some embodiments, the Gmd-binding antibody has at least 2, at least 3, or at least 4, of the above characteristics.

In some embodiments, the Gmd-binding antibody (e.g., a full length antibody or a Gmd-binding antibody fragment) is an antibody that specifically binds a Gmd fragment consisting of the amino acid sequence of SEQ ID NO:72 (e.g., a fragment consisting of amino acid residues 59-91 of SEQ ID NO:72). In some embodiments, the Gmd-binding antibody is a murine antibody, a humanized antibody, a chimeric antibody, a monoclonal antibody, a polyclonal antibody, a recombinant antibody, a multispecific antibody, or any combination thereof. In some embodiments, the Gmd-binding antibody is an Fv fragment, an Fab fragment, an F(ab′)₂ fragment, an Fab′ fragment, a dsFv fragment, an scFv fragment, or an sc (Fv) 2 fragment.

In additional embodiments, the Gmd-binding antibody (e.g., a full length antibody or a Gmd-binding antibody fragment) cross-blocks or competes for binding to Gmd with an antibody comprising a VH and a VL pair disclosed in Table 1 and wherein the Gmd-binding antibody does not comprise the VH sequence of SEQ ID NO:9, the VL sequence of SEQ ID NO:41, or the VH sequence of SEQ ID NO:9 and the VL sequence of SEQ ID NO:41. In additional embodiments, the Gmd-binding antibody cross-blocks or competes for binding to Gmd with an antibody comprising a VH having the amino acid sequence of SEQ ID NO:3 and a VL having the amino acid sequence of SEQ ID NO:36, wherein the Gmd-binding antibody does not comprise the VH sequence of SEQ ID NO:9, the VL sequence of SEQ ID NO:41, or the VH sequence of SEQ ID NO:9 and the VL sequence of SEQ ID NO: 41.

In some embodiments, the Gmd-binding antibody comprises a VH of SEQ ID NO:1-7 or 8. In additional embodiments, the Gmd-binding antibody comprises a VL of SEQ ID NO:33-39, or 40. In further embodiments, the Gmd-binding antibody comprises a VH of SEQ ID NO: 1-7 or 8; and a VL of 33-39, or 40. In additional embodiments, a Gmd-binding antibody binds to the same epitope as a Gmd-binding antibody disclosed in Table 1 and does not comprise the VH sequence of SEQ ID NO: 9, the VL sequence of SEQ ID NO:41, or the VH sequence of SEQ ID NO:9 and the VL sequence of SEQ ID NO:41. The ability of a Gmd-binding antibody to compete for binding with and/or bind the same epitope of Gmd as a reference Gmd-binding antibody can readily be determined using techniques disclosed herein or otherwise known in the art. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%. In some embodiments, the Gmd-binding antibody has 2, 3, or 4 of the above characteristics. In some embodiments, the Gmd-binding antibody has at least 2, at least 3, or at least 4, of the above characteristics.

In one embodiment, the Gmd-binding antibody (e.g., a full length antibody or a Gmd-binding antibody fragment) comprises a VH and a VL pair disclosed in Table 1. In one embodiment, the Gmd-binding comprises a VH and a VL pair having a VH of SEQ ID NO:1-7 or 8 and a VL of SEQ ID NO:33-39, or 40. In further embodiments, the Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%. In some embodiments, the Gmd-binding antibody has 2, 3, or 4 of the above characteristics. In some embodiments, the Gmd-binding antibody has at least 2, at least 3, or at least 4, of the above characteristics.

In one embodiment, the Gmd-binding antibody (e.g., a full length antibody or a Gmd-binding antibody fragment) comprises a VH and a VL selected from the group consisting of: (a) a VH sequence of SEQ ID NO: 1 and a VL sequence of SEQ ID NO:34; (b) a VH sequence of SEQ ID NO:1 and a VL sequence of SEQ ID NO:35; (c) a VH sequence of SEQ ID NO:2 and a VL sequence of SEQ ID NO: 34; (d) a VH sequence of SEQ ID NO:2 and a VL sequence of SEQ ID NO:35; (e) a VH sequence of SEQ ID NO:3 and a VL sequence of SEQ ID NO:33; (f) a VH sequence of SEQ ID NO:3 and a VL sequence of SEQ ID NO:35; (g) a VH sequence of SEQ ID NO:3, and a VL sequence of SEQ ID NO:36; and (h) a VH sequence of SEQ ID NO:3 and a VL sequence of SEQ ID NO:38; (i) a VH sequence of SEQ ID NO:3 and a VL sequence of SEQ ID NO:39; (j) a VH sequence of SEQ ID NO: 4 and a VL sequence of SEQ ID NO:39; (k) a VH sequence of SEQ ID NO:5 and a VL sequence of SEQ ID NO:35; (l) a VH sequence of SEQ ID NO:5 and a VL sequence of SEQ ID NO:36; (m) a VH sequence of SEQ ID NO:5 and a VL sequence of SEQ ID NO:38; (n) a VH sequence of SEQ ID NO: 5 and a VL sequence of SEQ ID NO:39; (o) a VH sequence of SEQ ID NO:6, and a VL sequence of SEQ ID NO:35; (p) a VH sequence of SEQ ID NO:6 and a VL sequence of SEQ ID NO:36; (q) a VH sequence of SEQ ID NO:6 and a VL sequence of SEQ ID NO:38; (r) a VH sequence of SEQ ID NO: 7 and a VL sequence of SEQ ID NO:34; (s) a VH sequence of SEQ ID NO:7 and a VL sequence of SEQ ID NO:35; (t) a VH sequence of SEQ ID NO:8 and a VL sequence of SEQ ID NO:33; (u) a VH sequence of SEQ ID NO:8 and a VL sequence of SEQ ID NO:34; (v) a VH sequence of SEQ ID NO: 8 and a VL sequence of SEQ ID NO:35; (w) a VH sequence of SEQ ID NO:8, and a VL sequence of SEQ ID NO:36; (x) a VH sequence of SEQ ID NO:8 and a VL sequence of SEQ ID NO:37; (y) a VH sequence of SEQ ID NO:8 and a VL sequence of SEQ ID NO:38; and (2) a VH sequence of SEQ ID NO: 8 and a VL sequence of SEQ ID NO:40.

In one embodiment, the Gmd-binding antibody (e.g., a full length antibody or a Gmd-binding antibody fragment) comprises a VH and a VL containing a VH of SEQ ID NO:1, and a VL of SEQ ID NO:33-39, or 40. In one embodiment, the Gmd-binding antibody comprises a VH of SEQ ID NO: 1 and a VL of SEQ ID NO:34 or 35. In one embodiment, the Gmd-binding antibody comprises a VH of SEQ ID NO: 1 and a VL of SEQ ID NO:34. In a further embodiment, the Gmd-binding antibody comprises a VH of SEQ ID NO: 1 and a VL sequence of SEQ ID NO:35.

In another embodiment, the Gmd-binding antibody comprises a VH of SEQ ID NO:2 and a VL of SEQ ID NO:33-39, or 40. In one embodiment, the Gmd-binding antibody comprises a VH of SEQ ID NO:2 and a VL of SEQ ID NO:34, 35, or 37. In one embodiment, the Gmd-binding antibody comprises a VH of SEQ ID NO:2 and a VL of SEQ ID NO:34. In another embodiment, the Gmd-binding antibody comprises a VH of SEQ ID NO:2 and a VL of SEQ ID NO:35. In a further embodiment, the Gmd-binding antibody comprises a VH of SEQ ID NO:2 and a VL of SEQ ID NO:37.

In an additional further embodiment, the Gmd-binding antibody (e.g., a full length antibody or a Gmd-binding antibody fragment) comprises a VH of SEQ ID NO:3 and a VL of SEQ ID NO:33-39, or 40. In one embodiment, the Gmd-binding antibody comprises a VH of SEQ ID NO:3 and a VL of SEQ ID NO:33, 35, 36, 38, or 39. In one embodiment, the Gmd-binding antibody comprises a VH of SEQ ID NO:3 and a VL of SEQ ID NO:33. In another embodiment, the Gmd-binding antibody comprises a VH of SEQ ID NO:3 and a VL of SEQ ID NO:35. In another embodiment, the Gmd-binding antibody comprises a VH of SEQ ID NO:3 and a VL of SEQ ID NO:36. In another embodiment, the Gmd-binding antibody comprises a VH of SEQ ID NO:3 and a VL of SEQ ID NO:38. In a further embodiment, the Gmd-binding antibody comprises a VH of SEQ ID NO:3 and a VL of SEQ ID NO:39.

In a further embodiment, the Gmd-binding antibody (e.g., a full length antibody or a Gmd-binding antibody fragment) comprises a VH of SEQ ID NO:4 and a VL of SEQ ID NO:33-39, or 40. In a further embodiment, the Gmd-binding antibody (e.g., a full length antibody or a Gmd-binding antibody fragment) comprises a VH and a VL containing a VH of SEQ ID NO:4, and a VL of SEQ ID NO: 39.

In a further embodiment, the Gmd-binding antibody (e.g., a full length antibody or a Gmd-binding antibody fragment) comprises a VH of SEQ ID NO:5 and a VL of SEQ ID NO:33-39, or 40. In one embodiment, the Gmd-binding antibody comprises a VH of SEQ ID NO:5 and a VL of SEQ ID NO: 35, 36, 38, or 39. In one embodiment, the Gmd-binding antibody comprises a VH of SEQ ID NO: 5 and a VL of SEQ ID NO:35. In another embodiment, the Gmd-binding antibody comprises a VH of SEQ ID NO:5 and a VL of SEQ ID NO:36. In another embodiment, the Gmd-binding antibody comprises a VH of SEQ ID NO:5 and a VL of SEQ ID NO:38. In a further embodiment, the Gmd-binding antibody comprises a VH of SEQ ID NO:5 and a VL of SEQ ID NO:39.

In an additional embodiment, the Gmd-binding antibody (e.g., a full length antibody or a Gmd-binding antibody fragment) comprises a VH of SEQ ID NO:6 and a VL of SEQ ID NO:33-39, or 40. In one embodiment, the Gmd-binding antibody comprises a VH of SEQ ID NO:6 and a VL of SEQ ID NO:35, 36, or 38. In one embodiment, the Gmd-binding antibody comprises a VH of SEQ ID NO: 6 and a VL of SEQ ID NO:35. In another embodiment, the Gmd-binding antibody comprises a VH of SEQ ID NO:6 and a VL of SEQ ID NO:36. In a further embodiment, the Gmd-binding antibody comprises a VH of SEQ ID NO:6 and a VL of SEQ ID NO:38.

In a further embodiment, the Gmd-binding antibody (e.g., a full length antibody or a Gmd-binding antibody fragment) comprises a VH of SEQ ID NO:7 and a VL of SEQ ID NO:33-39, or 40. In one embodiment, the Gmd-binding antibody comprises a VH of SEQ ID NO:7 and a VL of SEQ ID NO: 34 or 35. In one embodiment, the Gmd-binding antibody comprises a VH of SEQ ID NO:7 and a VL of SEQ ID NO:34. In a further embodiment, the Gmd-binding antibody comprises a VH of SEQ ID NO:7 and a VL of SEQ ID NO:35.

In another embodiment, the Gmd-binding antibody (e.g., a full length antibody or a Gmd-binding antibody fragment) comprises a VH of SEQ ID NO:8 and a VL of SEQ ID NO:33-39, or 40. In one embodiment, the Gmd-binding antibody comprises a VH of SEQ ID NO:3 and a VL of SEQ ID NO: 33-38, or 40. In one embodiment, the Gmd-binding antibody comprises a VH of SEQ ID NO:8 and a VL of SEQ ID NO:33. In another embodiment, the Gmd-binding antibody comprises a VH of SEQ ID NO:8 and a VL of SEQ ID NO:34. In another embodiment, the Gmd-binding antibody comprises a VH of SEQ ID NO:8 and a VL of SEQ ID NO:35. In another embodiment, the Gmd-binding antibody comprises a VH of SEQ ID NO:8 and a VL of SEQ ID NO:36. In another embodiment, the Gmd-binding antibody comprises a VH of SEQ ID NO:8 and a VL of SEQ ID NO:37. In another embodiment, the Gmd-binding antibody comprises a VH of SEQ ID NO:8 and a VL of SEQ ID NO:38. In a further embodiment, the Gmd-binding antibody comprises a VH of SEQ ID NO:8 and a VL of SEQ ID NO:40.

In some embodiments, the Gmd-binding antibody (e.g., a full length antibody or a Gmd-binding antibody fragment) comprises a VH sequence of SEQ ID NO:1 and a VL sequence of SEQ ID NO: 34.

In some embodiments, the Gmd-binding antibody (e.g., a full length antibody or a Gmd-binding antibody fragment) comprises a VH sequence of SEQ ID NO:1 and a VL sequence of SEQ ID NO: 35.

In some embodiments, the Gmd-binding antibody (e.g., a full length antibody or a Gmd-binding antibody fragment) comprises a VH sequence of SEQ ID NO:2 and a VL sequence of SEQ ID NO: 34.

In some embodiments, the Gmd-binding antibody (e.g., a full length antibody or a Gmd-binding antibody fragment) comprises a VH sequence of SEQ ID NO:2 and a VL sequence of SEQ ID NO: 35.

In some embodiments, the Gmd-binding antibody (e.g., a full length antibody or a Gmd-binding antibody fragment) comprises a VH sequence of SEQ ID NO:3 and a VL sequence of SEQ ID NO: 33.

In some embodiments, the Gmd-binding antibody (e.g., a full length antibody or a Gmd-binding antibody fragment) comprises a VH sequence of SEQ ID NO:3 and a VL sequence of SEQ ID NO: 35.

In some embodiments, the Gmd-binding antibody (e.g., a full length antibody or a Gmd-binding antibody fragment) comprises a VH sequence of SEQ ID NO:3 and a VL sequence of SEQ ID NO: 36.

In some embodiments, the Gmd-binding antibody (e.g., a full length antibody or a Gmd-binding antibody fragment) comprises a VH sequence of SEQ ID NO:3 and a VL sequence of SEQ ID NO: 38.

In some embodiments, the Gmd-binding antibody (e.g., a full length antibody or a Gmd-binding antibody fragment) comprises a VH sequence of SEQ ID NO:3 and a VL sequence of SEQ ID NO: 39.

In some embodiments, the Gmd-binding antibody (e.g., a full length antibody or a Gmd-binding antibody fragment) comprises a VH sequence of SEQ ID NO:4 and a VL sequence of SEQ ID NO: 39.

In some embodiments, the Gmd-binding antibody (e.g., a full length antibody or a Gmd-binding antibody fragment) comprises a VH sequence of SEQ ID NO:5 and a VL sequence of SEQ ID NO: 35.

In some embodiments, the Gmd-binding antibody (e.g., a full length antibody or a Gmd-binding antibody fragment) comprises a VH sequence of SEQ ID NO:5 and a VL sequence of SEQ ID NO: 36.

In some embodiments, the Gmd-binding antibody (e.g., a full length antibody or a Gmd-binding antibody fragment) comprises a VH sequence of SEQ ID NO:5 and a VL sequence of SEQ ID NO: 38.

In some embodiments, the Gmd-binding antibody (e.g., a full length antibody or a Gmd-binding antibody fragment) comprises a VH sequence of SEQ ID NO:5 and a VL sequence of SEQ ID NO: 39.

In some embodiments, the Gmd-binding antibody (e.g., a full length antibody or a Gmd-binding antibody fragment) comprises a VH sequence of SEQ ID NO:6 and a VL sequence of SEQ ID NO: 35.

In some embodiments, the Gmd-binding antibody (e.g., a full length antibody or a Gmd-binding antibody fragment) comprises a VH sequence of SEQ ID NO:6 and a VL sequence of SEQ ID NO: 36.

In some embodiments, the Gmd-binding antibody (e.g., a full length antibody or a Gmd-binding antibody fragment) comprises a VH sequence of SEQ ID NO:6 and a VL sequence of SEQ ID NO: 38.

In some embodiments, the Gmd-binding antibody (e.g., a full length antibody or a Gmd-binding antibody fragment) comprises a VH sequence of SEQ ID NO:7 and a VL sequence of SEQ ID NO: 34.

In some embodiments, the Gmd-binding antibody (e.g., a full length antibody or a Gmd-binding antibody fragment) comprises a VH sequence of SEQ ID NO:7 and a VL sequence of SEQ ID NO: 35.

In some embodiments, the Gmd-binding antibody comprises a VH sequence of SEQ ID NO: 8 and a VL sequence of SEQ ID NO:33.

In some embodiments, the Gmd-binding antibody (e.g., a full length antibody or a Gmd-binding antibody fragment) comprises a VH sequence of SEQ ID NO:8 and a VL sequence of SEQ ID NO: 34.

In some embodiments, the Gmd-binding antibody (e.g., a full length antibody or a Gmd-binding antibody fragment) comprises a VH sequence of SEQ ID NO:8 and a VL sequence of SEQ ID NO: 35.

In some embodiments, the Gmd-binding antibody (e.g., a full length antibody or a Gmd-binding antibody fragment) comprises a VH sequence of SEQ ID NO:8 and a VL sequence of SEQ ID NO: 36.

In some embodiments, the Gmd-binding antibody is a full length antibody. In some embodiments, the Gmd-binding antibody is a monoclonal antibody, a recombinant antibody, a human antibody, a humanized antibody, a chimeric antibody, a bi-specific antibody, or a multi-specific antibody. In some embodiments, the Gmd-binding antibody is a Gmd-binding antibody fragment. In some embodiments, the Gmd-binding antibody fragment is selected from the group consisting of a Fab, Fab′, F(ab′)₂. Fv, diabody, DART, and a single chain antibody molecule (e.g., a BiTE).

The disclosure further provides Gmd-binding antibodies (e.g., full length antibodies or a Gmd-binding antibody fragments) that are conjugated to a heterologous agent. In certain embodiments, the heterologous agent is an antimicrobial agent, a therapeutic agent, a prodrug, a peptide, a protein, an enzyme, a lipid, a biological response modifier, a pharmaceutical agent, a lymphokine, a heterologous antibody or antibody fragment, a detectable label, or a polyethylene glycol (PEG). Heteroconjugate Gmd-binding antibodies (are discussed in more detail elsewhere herein.

### Preparation of Gmd-Binding Antibodies

Gmd-binding antibodies (e.g., full length antibodies and Gmd-binding antibody fragments) can be readily prepared using known techniques. For example, monoclonal Gmd-binding antibodies can be prepared using techniques known in the art, including hybridoma methods, such as those described by Kohler and Milstein, Nature 256:495-497 (1975). Using the hybridoma method, a mouse, hamster, or other appropriate host animal, is immunized as described above to elicit the production by lymphocytes of antibodies that will specifically bind to an immunizing antigen. Lymphocytes can also be immunized in vitro. Following immunization, the lymphocytes are isolated and fused with a suitable myeloma cell line to form hybridoma cells that can then be selected away from unfused lymphocytes and myeloma cells. Hybridomas that produce monoclonal antibodies directed specifically against a Gmd fragment consisting of the amino acid sequence of SEQ ID NO:72 (e.g., a fragment consisting of amino acid residues 59-91 of SEQ ID NO:72), as determined by immunoprecipitation, immunoblotting, or by an in vitro binding assay (e.g., radioimmunoassay (RIA); enzyme-linked immunosorbent assay (ELISA)) can then be propagated either in in vitro culture using standard methods (see, e.g., Goding, Monoclonal Antibodies: principles and Practice, Academic Press, 1986) or in vivo as ascites tumors in an animal. The monoclonal antibodies can then be purified from the culture medium or ascites fluid as described for polyclonal antibodies above.

The disclosed Gmd-binding antibodies can also be made using recombinant DNA methods as described in U.S. Pat. No. 4,816,567, wherein the polynucleotides encoding a monoclonal antibody are isolated from mature B-cells or a hybridoma cell, such as by RT-PCR using oligonucleotide primers that specifically amplify the genes encoding the heavy and light chains of the Gmd-binding antibody, and their sequence is determined using known procedures. The isolated polynucleotides encoding the heavy and light chains are then cloned into suitable expression vectors, which when transfected into host cells such as E. coli cells, simian COS cells, Chinese hamster ovary (CHO) cells, Per.C6 cells, or myeloma cells (e.g., NS0 cells) that do not otherwise produce immunoglobulin protein, monoclonal antibodies are generated by the host cells. Recombinant anti-Gmd monoclonal antibodies can also readily be isolated from phage display libraries expressing CDRs of the desired species using known techniques (see, e.g., McCafferty et al., Nature 348:552-554 (1990); Clackson et al., Nature 352:624-628 (1991); and Marks et al., J. Mol. Biol. 222:581-597 (1991)).

The Gmd-binding antibodies can optionally be humanized, resurfaced, and engineered to display high affinity for the Gmd antigen and other favorable biological properties. For example, a humanized (or human) Gmd-binding antibody, can readily be designed and prepared using commonly available three-dimensional immunoglobulin modeling and known procedures for selecting framework (FW) residues, consensus sequences, and germline sequences to provide a desired antibody characteristic, such as increased affinity for Gmd.

Affinity maturation strategies and chain shuffling strategies are known in the art and can be employed to generate high affinity Gmd-binding antibodies as well as derivatives and variants of the Gmd-binding antibodies disclosed herein. See, e.g., Marks et al., Bio/Technology 10:779-783 (1992), which is herein incorporated by reference in its entirety. An additional strategy for generating high affinity Gmd-binding antibodies as well as derivatives and variants of the Gmd-binding antibodies disclosed herein is to generate novel VH or VL regions carrying CDR-derived sequences of the disclosure using random mutagenesis of one or more selected VH and/or VL coding sequences to generate mutations within the entire variable domain. Such a technique that uses error-prone PCR is described by Gram et al. (PNAS USA 89:3576-3580 (1992)). In some embodiments, one or two amino acid substitutions are made within a set of VH CDRs and/or VL CDRs. A further strategy uses direct mutagenesis to CDR regions of VH or VL nucleic acids that encode Gmd-binding antibodies disclosed herein. Examples of such techniques are disclosed by Barbas et al. (PNAS USA 91:3809-3813 (1994)) and Schier et al. (J. Mol. Biol. 263:551-567 (1996)).

Humanization, resurfacing or engineering of Gmd-binding antibodies of the disclosure can be performed using any known method including, but not limited to, those described in Jones et al., Nature 321:522 (1986); Riechmann et al., Nature 332:323 (1988); Verhoeyen et al., Science 239:1534 (1988); Sims et al., J. Immunol. 151:2296 (1993); Chothia et al., J. Mol. Biol. 196:901 (1987); Carter et al., PNAS USA 89:4285 (1992); Presta et al., J. Immunol. 151:2623 (1993); U.S. Pat. Nos. 5,639,641, 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539, 4,816,567, 7,557,189, 7,538,195, and 7,342,110; Intl. Appl. Nos. PCT/US98/16280; PCT/US96/18978; PCT/US91/09630; PCT/US91/05939; PCT/US94/01234; PCT/GB89/01334; PCT/GB91/01134; PCT/GB92/01755; Intl. Appl. Publ. Nos. WO90/14443, WO90/14424, and WO90/14430; and EP Pat. Publ. No. EP 229246; each of which is herein incorporated by reference in is entirely. Likewise, known assays are available for readily selecting anti-Gmd-antibodies displaying desirable features (e.g., assays for determining binding affinity to Gmd; cross-blocking assays such as the BIACORE®-based Gmd-binding antibody competition binding assays described herein).

Methods for engineering, humanizing or resurfacing non-human or human antibodies can also be used and are known in the art. A humanized, resurfaced or similarly engineered antibody can have one or more amino acid residues from a source that is non-human, e.g., but not limited to, mouse, rat, rabbit, non-human primate or other mammal. These non-human amino acid residues are replaced by residues that are often referred to as “import” residues, which are typically taken from an “import” variable, constant or other domain of a known human sequence. Such imported sequences can be used to reduce immunogenicity or reduce, enhance or modify binding, affinity, on-rate, off-rate, avidity, specificity, half-life, or any other suitable characteristic, as known in the art. Preferably, part or all of the non-human or human CDR sequences are maintained while the non-human sequences of the variable and constant regions can be replaced with human or other amino acids.

Nucleic acid(s) encoding a Gmd-binding antibody (e.g., a full length antibody or a Gmd-binding antibody fragment) can further be modified in a number of different manners using recombinant DNA technology to generate alternative antibodies. In some embodiments, nucleic acid(s) encoding the constant domains of the light and heavy chains of, for example, a mouse monoclonal antibody can be substituted (a) for those coding regions of, for example, a human antibody to generate a chimeric antibody or (b) for non-immunoglobulin encoding nucleic acid(s) to generate a fusion antibody. In some embodiments, the constant regions are truncated or removed to generate the desired antibody fragment of a monoclonal antibody. Site-directed or high-density mutagenesis of the variable region coding sequence can be used to optimize specificity, affinity, etc. of a monoclonal antibody.

Anti-Gmd human antibodies can be directly prepared using any of the numerous techniques known in the art. (See, e.g., Cole et al., Monoclonal Antibodies and Cancer Therapy, Alan R. Liss, p. 77 (1985); Boemer et al., J. Immunol. 147(1):86-95 (1991); and U.S. Pat. No. 5,750,373). Similarly, human Gmd-binding antibodies can readily be obtained from immortalized human B lymphocyte immunized in vitro or isolated from an immunized individual that produces an antibody directed against Gmd.

Human Gmd-binding antibodies can also be selected from a phage library that expresses human antibodies, as described, for example, in Vaughan et al., Nat. Biotech. 14:309-314 (1996), Sheets et al., PNAS 95:6157-6162 (1998), Hoogenboom and Winter, J. Mol. Biol. 227:381 (1991), and Marks et al., J. Mol. Biol. 222:581 (1991). Techniques for the generation and screening of antibody phage libraries are also described in U.S. Pat. Nos. 5,969,108, 6,172,197, 5,885,793, 6,521,404, 6,544,731, 6,555,313, 6,582,915, 6,593,081, 6,300,064, 6,653,068, 6,706,484, and 7,264,963; and Rothe et al., J. Mol. Biol. 376(4):1182-1200 (2008), each of which is herein incorporated by reference in its entirety.

Human Gmd-binding antibodies can also be made in transgenic mice containing human immunoglobulin loci that are capable upon immunization of producing human antibodies in the absence of endogenous immunoglobulin production. This approach is described for example, in U.S. Pat. Nos. 5,545,807, 5,545,806, 5,569,825, 5,625,126, 5,633,425, and 5,661,016.

Human Gmd-binding antibodies can also be selected and/or isolated from yeast-based antibody presentation libraries, as disclosed in, for example, Intl. Appl. Publ. No. WO012/009568, WO09/036379, WO10/105256, and WO03/074679; and U.S. Appl. Publ. No. US2002/0177170, the contents of each of which is herein incorporated by reference in its entirety. Such libraries are designed in silico to be reflective of the diversity afforded by the human preimmune repertoire.

Alternatively, Gmd-binding antibodies may be selected from a yeast-displayed antibody library see, for example: Blaise et al., Gene 342(2):211-218 (2004); Boder et al., Nat Biotechnol. 15(6):553-557 (1997); Kuroda et al., Biotechnol. Lett. 33(1):1-9 (2011). Review; Lauer et al., J. Pharm. Sci. 101(1):102-15 (2012); Orcutt K. D. and Wittrup K. D. Antibody Engineering, yeast display and selection 207-233 (2010); Rakestraw et al., Protein Eng. Des. Sel. 24(6):525-30 (2011); and U.S. Pat. Nos. 6,423,538, 6,696,251, and 6,699,658.

Various techniques are known for the production of antigen-binding antibody fragments. Traditionally, these fragments are derived via proteolytic digestion of intact antibodies (see, e.g., Morimoto et al., J. Biochem. Biophys. Meth. 24:107-117 (1993); and Brennan et al., Science 229:81 (1985)). In certain embodiments, a Gmd-binding antibody fragments produced recombinantly. Fab, Fv, and scFv antibody fragments can all be expressed in and secreted from E. coli or other host cells, thus allowing the production of large amounts of these fragments. Such a Gmd-binding antibody fragments can additionally be isolated from the Gmd-binding antibody phage libraries discussed above. In some embodiments, the Gmd-binding antibody fragment is a linear antibody as described in U.S. Pat. No. 5,641,870. Other techniques for the production of antigen-binding antibody fragments are known in the art.

Known techniques can be readily adapted for the production of single-chain antibodies that bind Gmd (see, e.g., U.S. Pat. No. 4,946,778). In addition, known methods can routinely be adapted for the construction of Fab expression libraries (see, e.g., Huse et al., Science 246:1275-1281 (1989)) to allow rapid and effective identification of monoclonal Fab fragments with the desired specificity for Gmd. Gmd-binding antibody fragments can be produced by techniques known in the art including, but not limited to: (a) a F(ab′)₂ fragment produced by pepsin digestion of an antibody; (b) a Fab fragment generated by reducing the disulfide bridges of an F(ab′)₂ fragment, (c) a Fab fragment generated by the treatment of the Gmd-binding antibody with papain and a reducing agent, and (d) Fv fragments.

In certain embodiments, a Gmd-binding antibody (e.g., a full length antibody or a Gmd-binding antibody fragment) can be modified in order to increase its serum half-life. This can be achieved, for example, by incorporation of a salvage receptor binding epitope into the Gmd-binding antibody by mutation of an appropriate region in the Gmd-binding antibody or by incorporating the salvage receptor epitope into a peptide tag that is then fused to the Gmd-binding antibody at either end or in the middle (e.g., by DNA or peptide synthesis). Other methods to increase the serum half-life of a Gmd-binding antibody, e.g., conjugation to a heterologous molecule such as PEG are known in the art.

Heteroconjugate Gmd-binding antibodies (e.g., full length antibodies and Gmd-binding antibody fragments) are also within the scope of the disclosure. Heteroconjugate Gmd-binding antibodies are composed of two covalently joined proteins. It is contemplated that the heteroconjugate Gmd-binding antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

Gmd-binding antibodies (e.g., full length antibodies and Gmd-binding antibody fragments) can comprise any type of variable region that provides for the association of the Gmd-binding antibody with Gmd. Such variable region can comprise or be derived from any mammal that can be induced to mount a humoral response and generate immunoglobulins against the Gmd antigen. The variable region of a Gmd-binding antibody can be, for example, of human, murine, non-human primate (e.g., cynomolgus monkeys, macaques, etc.) or lupine origin. In some embodiments, both the variable and constant regions of the modified Gmd-binding antibodies are human. In other embodiments, the variable regions of compatible antibodies (usually derived from a non-human source) can be engineered or specifically tailored to improve the binding properties or reduce the immunogenicity of the molecule. In this respect, variable regions useful according to the disclosure can be humanized or otherwise altered through the inclusion of imported amino acid sequences using affinity maturation, mutagenesis procedures, chain shuffling strategies and/or other methods described herein or otherwise know in the art.

In certain embodiments, the variable domains in both the heavy and light chains of a Gmd-binding antibody are altered by at least partial replacement of one or more CDRs and/or by partial framework region replacement and sequence changing. Although the CDRs can be derived from an antibody of the same class or even subclass as the Gmd-binding antibody from which the framework regions are derived, it is envisaged that the CDRs will be derived from an antibody of different class and in certain embodiments, from an antibody from a different species. It is not necessary to replace all of the CDRs with the complete CDRs from the donor variable region to transfer the antigen-binding capacity of one variable domain to another. Rather, it is only necessary to transfer those residues that are necessary to maintain the activity of the antigen-binding site. It is well within the competence of those of ordinary skill in the art, to routinely obtain a functional antibody with reduced immunogenicity. See, e.g., U.S. Pat. Nos. 5,585,089, 5,693,761, and 5,693,762.

Alterations to the variable region notwithstanding, those of ordinary skill in the art will appreciate that the modified Gmd-binding antibody of the disclosure will comprise antibodies in which at least a fraction of one or more of the constant region domains has been deleted or otherwise altered so as to provide desired biochemical characteristics such as decreased ADCC or increased serum half-life when compared with an antibody of approximately the same immunogenicity comprising a native or unaltered constant region. In some embodiments, the constant region of the modified Gmd-binding antibody comprises a human constant region. Modifications to the constant region can include additions, deletions or substitutions of one or more amino acids in one or more domains. The modified Gmd-binding antibodies disclosed herein can comprise alterations or modifications to one or more of the three heavy chain constant domains (CH1, CH2 or CH3) and/or to the light chain constant domain (CL). In some embodiments, the modified Gmd-binding antibodies comprise constant regions wherein one or more domains are partially or entirely deleted are contemplated. In some embodiments, the modified Gmd-binding antibodies comprise domain deleted constructs or variants wherein the entire CH2 domain has been removed (ΔCH2 constructs). In some embodiments, the omitted constant region domain can be replaced by a short amino acid spacer (e.g., 10 residues) that provides some of the molecular flexibility typically imparted by the absent constant region.

It is generally understood that the constant region mediates several effector functions. For example, binding of the C1 component of complement to antibodies activates the complement system. Activation of complement is important in the opsonization and lysis of cell pathogens. The activation of complement also stimulates the inflammatory response and can also be involved in autoimmune hypersensitivity. Further, antibodies bind to cells via the Fc region, with a Fc receptor site on the Gmd-binding antibody Fc region binding to a Fc receptor (FcR) on a cell. There are a number of Fc receptors that are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production.

In certain embodiments, a Gmd-binding antibody has an altered effector function that, in turn, affects the biological profile of the administered Gmd-binding antibody. For example, the deletion or inactivation (through point mutations or other means) of a constant region domain can reduce Fc receptor binding of the circulating modified antibody. In other cases the constant region modifications, can moderate complement binding and thus reduce the serum half-life and nonspecific association of a conjugated cytotoxin. Yet other modifications of the constant region can be used to eliminate disulfide linkages or oligosaccharide moieties that allow for enhanced localization due to increased antigen specificity or antibody flexibility. Similarly, modifications to the constant region in accordance with this disclosure can easily be made using biochemical or molecular engineering techniques known to those of ordinary skill in the art.

In some embodiments, a Gmd-binding antibody (e.g., a full length antibody or a Gmd-binding antibody fragment) provided herein is a Gmd antibody that does not have one or more effector functions. For instance, in some embodiments, the Gmd-binding antibody has no antibody-dependent cellular cytoxicity (ADCC) activity and/or no complement-dependent cytoxicity (CDC) activity. In certain embodiments, the Gmd-binding antibody does not bind to an Fc receptor and/or complement factors. In certain embodiments, the Gmd-binding antibody has no effector function. Examples of Fc sequence engineering modifications that reduce or eliminate ADCC and/or CDC activity and Fc receptor and/or complement factor binding are described herein or otherwise know in the art, as are assays and procedures for testing the same.

In some embodiments, a Gmd-binding antibody is engineered to fuse the CH3 domain directly to the hinge region of the respective modified antibody. In other constructs a peptide spacer is inserted between the hinge region and the modified CH2 and/or CH3 domains. For example, compatible constructs can be expressed in which the CH2 domain has been deleted and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. Amino acid spacers can, in some cases, prove to be immunogenic and elicit an unwanted immune response against the construct. Accordingly, in certain embodiments, any spacer added to the construct can be relatively non-immunogenic, or even omitted altogether, so as to maintain the desired biochemical qualities of the modified Gmd-binding antibody.

In additional embodiments, Gmd-binding antibodies are modified by the partial deletion or substitution of a few or even a single amino acid in a constant region. For example, the mutation of a single amino acid in selected areas of the CH2 domain can be enough to substantially reduce Fc binding and thereby. Similarly one or more constant region domains that control the effector function (e.g., complement C1Q binding) can be fully or partially deleted. Such partial deletions of the constant regions can improve selected characteristics of the Gmd-binding antibody (e.g., serum half-life) while leaving other desirable functions associated with the corresponding constant region domain intact. In some embodiments, the constant region of the Gmd-binding antibody is modified through the mutation or substitution of one or more amino acids that enhances the profile of the resulting construct. In this respect it is possible to disrupt the activity provided by a conserved binding site (e.g., Fc binding) while substantially maintaining the configuration and immunogenic profile of the modified Gmd-binding antibody. The disclosure also provides a Gmd-binding antibody that contains the addition of one or more amino acids to the constant region to enhance desirable characteristics such, as decreasing or increasing effector function or providing attachments sites for one or more cytotoxin, labeling or carbohydrate moieties. In such embodiments, it can be desirable to insert or replicate specific sequences derived from selected constant region domains.

The disclosure also provides a Gmd-binding antibody (e.g., a full length antibody or a Gmd-binding antibody fragment) that is a variant to a Gmd-binding antibody disclosed in Table 1, but wherein the Gmd-binding antibody does not comprise the VH sequence of SEQ ID NO:9, the VL sequence of SEQ ID NO:41, or the VH sequence of SEQ ID NO:9 and the VL sequence of SEQ ID NO: 41. In particular embodiments, the variant Gmd-binding antibody has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody has at least 2, at least 3, or at least 4, of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%.

The provided Gmd-binding antibodies (e.g., full length antibodies and Gmd-binding antibody fragments), can be derivatized to contain additional chemical moieties known in the art for improving for example, the solubility, biological half-life, bioavailability, and to otherwise improve the stability, formulation and/or therapeutic properties of the Gmd-binding antibody. A non-exhaustive overview for such moieties can be found for example, in Remington's Pharmaceutical Sciences, 20th ed., Mack Publishing Co., Easton, PA (2000).

Nucleic Acids Encoding Gmd-Binding Antibodies and their Expression

Nucleic acid molecules and combinations of nucleic acid molecules that encode a Gmd-binding antibody (e.g., a full length antibody or a Gmd-binding antibody fragments) are also provided. In some embodiments, the nucleic acids molecules encode a Gmd-binding antibody, such as a full-length Gmd-binding antibody or a Gmd-binding antibody fragment. In further embodiments, the disclosure provides nucleic acid molecules that encode a variant or derivative of a full-length antibody or a Gmd-binding antibody fragment provided herein.

The nucleic acid molecules disclosed herein can be in the form of RNA or in the form of DNA. DNA includes cDNA, genomic DNA, and synthetic DNA; and can be double-stranded or single-stranded, and if single stranded can be the coding strand/or non-coding (anti-sense) strand. In certain embodiments, the nucleic acid molecule is isolated. In additional embodiments, a nucleic acid molecule is substantially pure. In some embodiments, the nucleic acid is cDNA or is derived from cDNA. In some embodiments, the nucleic acid is recombinantly produced.

In some embodiments, the nucleic acid molecule(s) comprise Gmd-binding protein (e.g., a full length antibody or a Gmd-binding antibody fragment) coding sequence(s) operably linked to sequence(s) that controls the expression of the coding sequence in a host cell or in vitro. In particular embodiments, the coding sequence is cDNA. The disclosure also relates to vectors containing nucleic acid molecules that comprise a Gmd-binding antibody coding sequence operably linked to a control sequence that controls the expression of the coding sequence in a host cell or in vitro.

In some embodiments, the nucleic acid molecule comprises a coding sequence for a Gmd-binding antibody (e.g., a full length antibody or a Gmd-binding antibody fragment) that is fused in the same reading frame to a heterologous polynucleotide sequence. In some embodiments, the heterologous polynucleotide sequence encodes a leader peptide sequence that facilitates the secretion of the expressed protein from the host cell transformed with the Gmd-binding antibody encoding nucleic acid molecule(s). A protein containing a leader sequence is referred to as a preprotein and can have the leader sequence cleaved by the host cell to form the mature form of the Gmd-binding antibody. Such leader peptide sequences and their use facilitating the secretion of recombinant proteins in host cells is generally known in the art. In additional embodiments, the heterologous polynucleotide sequence encodes additional 5′ amino acid residues that can function for example, to facilitate purification, and to add or improve protein stability and/or therapeutic or diagnostic properties of the recombinantly expressed Gmd-binding antibody.

In some embodiments, the disclosure provides isolated nucleic acids such as a Gmd-binding antibody encoding cDNA fragments, sufficient for use as a hybridization probe, PCR primer or sequencing primer.

In some embodiments, the nucleic acid molecules encode a Gmd-binding antibody (e.g., a full length antibody or a Gmd-binding antibody fragment) specifically binds a Gmd fragment consisting of the amino acid sequence of SEQ ID NO:72 (e.g., a fragment consisting of amino acid residues 59-91 of SEQ ID NO:72) and has at least one characteristic selected from the group consisting of: (a) inhibits in vitro growth of S. aureus; (b) inhibits in vivo growth of S. aureus; (c) promotes clumping (clustering) or inhibits cell division of S. aureus (e.g., Xen29); (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody has at least 2, at least 3, or at least 4, of the above characteristics. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In further embodiments, the Gmd-binding antibody inhibits in vivo growth of S. aureus by 20% to 100%, 30% to 100%, 40% to 100%, 50% to 100%, 60% to 100%, 70% to 100%, or 80% to 100%. In some embodiments, the encoded Gmd-binding antibody cross-blocks or competes for binding to Gmd with an antibody having a Gmd-binding VH and VL pair disclosed herein. In additional embodiments, the encoded Gmd-binding antibody binds to the same epitope of Gmd as an antibody disclosed herein. In some embodiments, the encoded Gmd-binding antibody cross-blocks or competes for binding to Gmd with a Gmd-binding antibody having a VH and VL pair disclosed herein. In additional embodiments, the encoded Gmd-binding antibody binds to the same epitope of Gmd as an antibody disclosed herein. In further embodiments, the nucleic acid molecules encode a Gmd-binding antibody that comprises a VH and a VL pair disclosed herein.

In some embodiments, the disclosure provides vectors and sets of vectors containing nucleic acids and sets of nucleic acids encoding a Gmd-binding antibody (e.g., a full length antibody or a Gmd-binding antibody fragment) provided herein. Host cells transformed with these nucleic acids, sets of nucleic acids, vectors, and sets of vectors are also provided, as are methods of making an using the Gmd-binding antibodies.

In certain embodiments, the nucleic acid(s) encoding the Gmd-binding antibodies comprise the coding sequence(s) for the mature Gmd-binding antibody (e.g., a full length antibody or a Gmd-binding antibody fragment) fused in the same reading frame to a marker sequence that allows, for example, for purification of the encoded polypeptide. For example, the marker sequence can be a hexahistidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used.

Nucleic acid variants encoding Gmd-binding antibodies (e.g., a full length antibodies or a Gmd-binding antibody fragments) are also provided. Nucleic acid variants can contain alterations in the coding regions, non-coding regions, or both. In some embodiments, the nucleic acid variants contain alterations that produce silent substitutions, additions, or deletions, but do not alter the properties or activities of the encoded polypeptide. In some embodiments, the nucleic acid variants are produced by silent substitutions due to the degeneracy of the genetic code. Nucleic acid variants can be produced for a variety of reasons, e.g., to optimize codon expression for a particular host (change codons in the human mRNA to those preferred by a bacterial host such as E. coli). Vectors and cells comprising the nucleic acids described herein are also provided.

In some embodiments, a nucleic acid sequence encoding a Gmd-binding antibody (e.g., a full length antibody or a Gmd-binding antibody fragment) is constructed by chemical synthesis using an oligonucleotide synthesizer. Such oligonucleotides can be designed based on the amino acid sequence of the desired polypeptide and codon optimization based on the host cell preferences. Standard methods can routinely be applied to synthesize an isolate polynucleotide sequences encoding Gmd-binding antibodies.

Once assembled (by synthesis, site-directed mutagenesis or another method), the nucleic acid sequences encoding Gmd-binding antibodies (e.g., full length antibodies and Gmd-binding antibody fragments) can routinely be operably linked to a control sequence appropriate for expression of the Gmd-binding antibody in a desired host. In some embodiments, the nucleic acid sequence(s) encoding a Gmd-binding antibody is inserted into one or more expression vectors and operably linked to a control sequence(s) appropriate for expression of the protein in a desired host. In order to obtain high expression levels of a transfected coding sequence in a host, the coding sequence can be operably linked to or associated with transcriptional and translational expression control sequences that are functional in the chosen expression host.

In certain embodiments, recombinant expression vectors are used to amplify and express DNA encoding a Gmd-binding antibody (e.g., a full length antibody and a Gmd-binding antibody fragment). Recombinant expression vectors are replicable DNA constructs which have synthetic or cDNA-derived DNA fragments encoding a polypeptide chain of a Gmd-binding antibody operably linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail below. Such regulatory elements can include an operator sequence to control transcription. The ability to replicate in a host, usually conferred by an origin of replication, and a selection gene to facilitate recognition of transformants can additionally be incorporated. DNA regions are operably linked when they are functionally related to each other. For example, DNA for a signal peptide (secretory leader) is operably linked to DNA for a polypeptide if it is expressed as a precursor which participates in the secretion of the polypeptide; a promoter is operably linked to a coding sequence if it controls the transcription of the sequence; or a ribosome binding site is operably linked to a coding sequence if it is positioned so as to permit translation. Structural elements intended for use in yeast expression systems include a leader sequence enabling extracellular secretion of translated protein by a host cell. Alternatively, where a recombinant protein is expressed without a leader or transport sequence, the protein can include an N-terminal methionine residue. This residue can optionally be subsequently cleaved from the expressed recombinant protein to provide a final protein. In certain embodiments, the disclosure provides a composition, e.g., a pharmaceutical composition, comprising a nucleic acid or vector as described above or elsewhere herein, optionally further comprising one or more carriers, diluents, excipients, or other additives.

In some embodiments, the disclosure provides a host cell comprising a nucleic acid molecule or combination of nucleic acid molecules or a vector as provided above, where the host cell can, in some instances express a Gmd-binding antibody (e.g., a full length antibody or a Gmd-binding antibody fragment) that binds to Gmd. In further embodiments, the disclosure provides a host cell transformed with a nucleic acid molecule or combination of nucleic acid molecules or a vector as provided above, where the host cell can, in some instances express a Gmd-binding antibody that binds to Gmd. Such host cells can be utilized in a method of making a Gmd-binding antibody as provided herein, where the method includes (a) culturing the host cell and (b) isolating the Gmd-binding antibodies expressed by the host cell.

Also provided is a host cell transformed with the nucleic acid molecule(s) (e.g., cDNAs) and/or the vectors disclosed herein. The disclosure also provides host cells transformed with the disclosed nucleic acid molecule or molecules operably linked to a control sequence(s) and optionally inserted into a vector. In some embodiments, the host cell is a mammalian host cell. In further embodiments, the mammalian host cell is a NS0 murine myeloma cell, a PER.C6® human cell, or a Chinese hamster ovary (CHO) cell. In other embodiments, the host cell is a hybridoma.

In additional embodiments, the disclosure provides a method of making a Gmd-binding antibody (e.g., a full length antibody or a Gmd-binding antibody fragment) provided herein comprising culturing a transformed host cell or a hybridoma disclosed herein under suitable conditions for producing the Gmd-binding antibody. The disclosure optionally provides isolating the Gmd-binding antibody secreted from the host cell. The disclosure also optionally provides the Gmd-binding antibody produced using this method and pharmaceutical compositions comprising the Gmd-binding antibody and a pharmaceutically acceptable carrier.

The choice of expression control sequence and expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR1, pBR322, pMB9 and their derivatives, and also wider host range plasmids, such as M13 and filamentous single-stranded DNA phages.

Suitable host cells for expression of a Gmd-binding antibody (e.g., a full length antibody and a Gmd-binding antibody fragment), include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin as described below. Cell-free translation systems could also be employed. Additional information regarding methods of protein production, including antibody production, can be found, e.g., in U.S. Appl. Publ. No. 2008/0187954, U.S. Pat. Nos. 6,413,746 and 6,660,501, and Intl. Appl. Publ. No. WO04/009823, each of which is herein incorporated by reference in its entirety.

Various mammalian or insect cell culture systems can also be advantageously employed to express recombinant Gmd-binding antibodies (e.g., full length antibodies and Gmd-binding antibody fragments). Expression of recombinant Gmd-binding antibodies in mammalian cells can be performed because such proteins are generally correctly folded, appropriately modified and completely functional. Examples of suitable mammalian host cell lines include HEK-293 and HEK-293T, the COS-7 lines of monkey kidney cells, described by Gluzman (Cell 23:175 (1981)), and other cell lines including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, BioTechnology 6:47 (1988).

Gmd-binding antibodies (e.g., full length antibodies and Gmd-binding antibody fragments) produced by a transformed host cell or hybridoma can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Gmd-binding antibodies can also be physically characterized using such techniques as proteolysis, nuclear magnetic resonance and x-ray crystallography.

For example, supernatants from systems that secrete recombinant Gmd-binding antibodies (e.g., full length antibodies and Gmd-binding antibody fragments) into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a Gmd-binding antibody. Some or all of the foregoing purification steps, in various combinations, can also routinely be employed to provide a homogeneous recombinant Gmd-binding antibody.

A recombinant Gmd-binding antibody (e.g., a full length antibody or a Gmd-binding antibody fragment) produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying target Gmd-binding antibodies such as full-length antibodies and antigen-binding antibody fragments also include, for example, those described in U.S. Appl. Publ. Nos. 2008/0312425, 2008/0177048, and 2009/0187005, each of which is incorporated herein by reference herein in its entirety.

### Methods of Use

The Gmd-binding antibodies (e.g., full length antibodies and Gmd-binding antibody fragments) of the present disclosure have applications in in vitro and in vivo diagnostic and therapeutic utilities. For example, the Gmd-binding antibodies can be administered to cells in culture, e.g., in vitro or in vivo, or in a subject, to treat, prevent or diagnose a variety of diseases or conditions. In some embodiments, the Gmd-binding antibodies (e.g., full length antibodies and Gmd-binding antibody fragments) are human antibodies, murine antibodies, or humanized antibodies.

The provided Gmd-binding antibodies (e.g., full length antibodies and Gmd-binding antibody fragments) are useful in a variety of applications including, but not limited to, diagnostic methods, prognostic methods, and methods for treating a Staphylococcus (e.g., S. aureus) infection, including infection-associated conditions.

In some embodiments, the disclosure provides a method of diagnosing or prognosing a Staphylococcus (e.g., S. aureus) infection that comprises contacting a patient sample with a Gmd-binding antibody disclosed herein, and detecting the presence of an immune complex between the Gmd-binding antibody and S. aureus or Gmd present in the sample, whereby the detection of the immune complex indicates the presence of Staphylococcus in the patient sample. In further embodiments, the patient sample is from the blood, serum, or plasma of a patient.

In another embodiment, the disclosure provides a method of determining the presence of Staphylococcus (e.g., S. aureus) in a sample that comprises contacting a biological sample with a Gmd-binding antibody disclosed herein, and detecting the presence of an immune complex formed between the Gmd-binding antibody and Staphylococcus or Gmd protein in the sample, whereby the detection of the immune complex indicates the presence of Staphylococcus in the sample. In further embodiments, the sample is a biological sample (e.g., a sample containing or derived from the blood, serum, or plasma of a subject). By “biological sample” is intended any biological sample obtained from an individual, cell line, tissue culture, or other source of cells potentially containing or expressing Gmd. Methods for obtaining tissue biopsies and body fluids from mammals are known in the art. The Gmd-binding antibodies (e.g., full length antibodies and Gmd-binding antibody fragments, and variants and derivatives thereof) can also be used to assay Gmd levels in a biological sample using classical immunohistological methods known to those of skill in the art (see, e.g., Jalkanen, et al., J. Cell. Biol. 101:976-985 (1985); Jalkanen et al., J. Cell Biol. 105:3087-3096 (1987)). Other antibody-based methods useful for detecting Gmd protein expression include immunoassays, such as the enzyme linked immunosorbent assay (ELISA), immunoprecipitation, or Western blotting. As used herein, “to assay Gmd levels in a biological sample” is intended qualitatively or quantitatively measuring or estimating the level of Gmd protein in a first biological sample either directly (e.g., by determining or estimating absolute protein level) or relatively (e.g., by comparing to the disease associated polypeptide level in a second (control) biological sample). The Gmd protein expression level in the first biological sample can be measured or estimated and compared to a standard Gmd protein level, the standard being taken from a second biological sample obtained from an individual not having the disorder or being determined by averaging levels from a population of individuals not having the disorder. As will be appreciated in the art, once the “standard” Gmd protein level is known, it can be used repeatedly as a standard for comparison.

In additional embodiments, the disclosure provides a pharmaceutical composition containing a Gmd-binding antibody provided herein and a pharmaceutically acceptable carrier. In some embodiments, the disclosure provides a pharmaceutical composition containing a Gmd-binding antibody and a pharmaceutically acceptable carrier, for use as a medicament. The disclosure also provides for the use of the pharmaceutical compositions disclosed herein for treating an infection and conditions associated with the infection of the microorganism that expresses Gmd such as, S. aureus. In some embodiments, the condition associated with the infection is fever, bacteremia, endocarditis, osteomyelitis, pneumonia, sepsis, dermonecrosis, or mastitis.

In some embodiments, the disclosure provides a method for treating a Staphylococcus (e.g., S. aureus) infection that comprises administering an effective amount of a disclosed Gmd-binding antibody or pharmaceutical composition to a subject having or at risk of having a Staphylococcus infection. In further embodiments, the Staphylococcus is S. aureus. In some embodiments, the method decreases the Staphylococcus titer in the subject. In further embodiments, the disclosure provides a method for treating a condition associated with a Staphylococcus infection in a patient. Conditions that may be treated in a subject using the provided methods include but are not limited to fever, fever, bacteremia, endocarditis, osteomyelitis, pneumonia, sepsis, dermonecrosis, and mastitis.

The disclosure also provides a method of inhibiting the growth of Staphylococcus (e.g., S. aureus) that comprises contacting a Gmd-binding antibody (e.g., a full length antibody or a Gmd-binding antibody fragment) and Staphylococcus. In some embodiments, Staphylococcus is S. aureus. In some embodiments, the method of inhibiting the growth of Staphylococcus is performed in vitro. In some embodiments, the Gmd-binding antibody inhibits in vitro growth of S. aureus by 20% to 100%, 30% to 100%, 40% to 100%, 50% to 100%, 60% to 100%, 70% to 100%, or 80% to 100%. In some embodiments, the method is performed in vivo. In some embodiments, the Gmd-binding antibody inhibits in vivo growth of S. aureus by 20% to 100%, 30% to 100%, 40% to 100%, 50% to 100%, 60% to 100%, 70% to 100%, or 80% to 100%. In some embodiments, the method comprises contacting a Gmd-binding antibody and an antibiotic-resistant Staphylococcus (e.g., S. aureus). In some embodiments, the method comprises contacting a Gmd-binding antibody and a methicillin-susceptible S. aureus. In some instances the method comprises contacting a Gmd-binding antibody and a methicillin-susceptible S. aureus (MSSA). In some instances the method comprises contacting a Gmd-binding antibody and a methicillin-resistant S. aureus (MRSA). In some instances the method comprises contacting a Gmd-binding antibody and a vancomycin resistant S. aureus (VRSA). In some instances the method comprises contacting a Gmd-binding antibody and a daptomycin-resistant S. aureus (DRSA). In some instances the method comprises contacting a Gmd-binding antibody and a linezolid-resistant S. aureus (LRSA). In some instances the method comprises contacting a Gmd-binding antibody and a bacteria with altered antibiotic sensitivity such as vancomycin intermediate-sensitivity S. aureus (VISA). In some embodiments, the disclosure provides a method of inhibiting growth of Staphylococcus in a subject that comprises administering an effective amount of a Gmd-binding antibody to a subject in need thereof.

In some embodiments, the disclosure provides a method for treating a Staphylococcus infection that comprises administering an effective amount of a Gmd-binding antibody (e.g., a full length antibody or a Gmd-binding antibody comprising the sequence of a VH and VL pair disclosed in Table 1) to a patient having or at risk of having a Staphylococcus infection. In further embodiments, the disclosure provides a method for treating a condition associated with the Staphylococcus infection. In some embodiments, the condition associated with the infection is selected from the group consisting of fever, bacteremia, endocarditis, osteomyelitis, pneumonia, sepsis, dermonecrosis, and maspatientis.

In further embodiments, the disclosure provides a method for treating an S. aureus infection that comprises administering an effective amount of a Gmd-binding antibody (e.g., a full length antibody or a Gmd-binding antibody fragment comprising the sequence of a VH and VL pair disclosed in Table 1) to a patient having or at risk of having a S. aureus infection. In further embodiments, the disclosure provides a method for treating a condition associated with the S. aureus infection. In some embodiments, the condition associated with the infection is selected from the group consisting of fever, bacteremia, endocarditis, osteomyelitis, pneumonia, sepsis, dermonecrosis, and maspatientis. In some embodiments, the S. aureus is resistant to one or more β-lactam antimicrobials. In some embodiments, the S. aureus is a member selected from the group consisting of: VRSA, DRSA, LRSA, and VISA.

In some embodiments, the disclosure provides a method for treating a Staphylococcus (e.g., S. aureus) bone or joint infection that comprises administering to a patient having or at risk of having a bone or joint infection, an effective amount of a Gmd-binding antibody disclosed herein (e.g., a full length antibody and a Gmd-binding antibody fragment having a sequence disclosed in Table 1). In further embodiments, the disclosure provides a method for treating osteomyelitis that comprises administering to a patient having or at risk of having a Staphylococcus bone or joint infection an effective amount of a Gmd-binding antibody a Gmd-binding antibody (e.g., a full length antibody or a Gmd-binding antibody comprising the sequence of a VH and VL pair disclosed in Table 1). In some embodiments, the infection is an S. aureus infection. In some embodiments, the S. aureus is MSSA. In additional embodiments, the S. aureus is resistant to an antibiotic. In some embodiments, the S. aureus is MRSA. In some embodiments, the S. aureus is resistant to one or more-lactam antimicrobials. In some embodiments, the S. aureus is a member selected from the group consisting of: VRSA, DRSA, LRSA, and VISA.

In additional embodiments, the disclosure provides a method of introducing a medical device (e.g., an orthopedic implant, dental implant, or ENT implant) or tissue graft into a patient that comprises administering to a patient in need of an medical device an effective amount of a Gmd-binding antibody disclosed herein (e.g., a full length antibody and a Gmd-binding antibody fragment), and introducing the orthopedic implant, tissue graft, or medical device into the patient. In some embodiments, the Gmd-binding antibody is administered to the patient before introducing the medical device (e.g., orthopedic implant). In some embodiments, the Gmd-binding antibody is administered to the patient at the same time as introducing the medical device. In some embodiments, the Gmd-binding antibody is administered to the patient after the medical device is introduced. In some embodiments, the Gmd-binding antibody is administered to the patient before and after the medical device is introduced into the patient.

In another aspect, the disclosure provides a method of screening a sample for the presence of Staphylococcus (e.g., S. aureus). In some embodiments, the sample is a blood sample, a serum sample, or a plasma sample. In some embodiments, the sample is a mucosa-associated lymphoid tissue (MALT) sample, a cerebrospinal fluid sample, an articular liquid sample, a pleural liquid sample, a saliva sample, a urine sample, or a tissue biopsy sample. In some embodiments, the method comprises contacting the sample with a Gmd-binding antibody (e.g., a full length antibody or a Gmd-binding antibody fragment) disclosed herein and detecting whether an immune complex forms between the Gmd-binding antibody and Staphylococcus or Gmd, wherein the detection of an immune complex indicates the presence of Staphylococcus in the sample. Methods for detecting the formation of an immune complex are known in the art and can be applied or routinely modified to detect the presence of Staphylococcus in a sample. In some embodiments, an immunoassay that uses one or more of the Gmd-binding antibodies disclosed herein is used to detect the presence of Staphylococcus in the sample. Various immunoassays described herein or otherwise known in the art may be used in performing the provided methods, including for example, common immunoassay methods such as latex agglutination methods, turbidimetric methods, radioimmunoassay methods (for example, RIA and RIMA), enzyme immunoassay methods (for example, ELISA and EIA), gel diffusion precipitation reaction, flow cytometry, Immunoelectrophoresis (for example Western blotting), dot blot methods, immunodiffusion assay, protein A immunoassay, fluorescent immunoassay (for example, FIA and IFMA), immunochromatography methods and antibody array methods. Various methods for labeling proteins are known in the art and may be used in performing the provided methods.

In some embodiments, a pharmaceutical composition contains a Gmd-binding antibody (e.g., a full length antibody or a Gmd-binding antibody fragment) and a pharmaceutically acceptable carrier, and the Gmd-binding antibody further comprises a labeling group or an effector group. A “label” refers to one or more elements, isotopes, or chemical compounds attached to enable the detection in a screen. Labels generally fall into three classes: (a) isotopic labels, which may be radioactive or heavy isotopes, (b) small molecule labels, which may include fluorescent and colorimetric dyes, or molecules such as biotin that enable other labeling methods, and (c) immune labels, which may be an epitope incorporated as a fusion partner that is recognized by an antibody, “Labeling group” refers to any detectable label. In some embodiments, the labeling group is coupled to the Gmd-binding antibody via a spacer (e.g., a peptide spacer) to reduce potential steric hindrance. Labels may be incorporated into the compound at any position and may be incorporated in vitro or in vivo during protein expression. Various methods for labeling proteins are known in the art and may be used in performing the provided methods. In additional embodiments, the labeling group is selected from the group consisting of: isotopic labels, magnetic labels, redox active moieties, optical dyes, biotinylated groups and polypeptide epitopes recognized by a secondary reporter. In some embodiments, the labeling group is a fluorescent protein such as a Green Fluorescent Protein or derivative thereof (e.g., enhanced GFP, blue fluorescent protein or derivative thereof (e.g., EBFP (Enhanced Blue Fluorescent Protein), EBFP2, Azurite, mKalama1, cyan fluorescent protein or derivative thereof (e.g., ECFP (Enhanced Cyan Fluorescent Protein), Cerulean, CyPet), yellow fluorescent protein or derivative thereof (e.g., YFP, Citrine, Venus, YPet). In some embodiments, the polypeptide epitope is a member selected from a biotin peptide, histidine peptide (his), hemagglutinin (HA), Flag, gold binding peptide. In additional embodiments, the effector group is selected from the group consisting of a radioisotope, radionucleotide, a toxin, a therapeutic and a chemotherapeutic agent.

Also provided are methods of inhibiting Gmd activity. In some embodiments, the method comprises contacting Gmd with a Gmd-binding antibody (e.g., a full length antibody and a Gmd-binding antibody fragment). In some instances the method is performed in vivo. In other instances, the method is performed in vitro. In some embodiments, the blocked Gmd activity is selected from (a) inhibits in vitro growth of S. aureus; (b) inhibits in vivo growth of S. aureus; (c) promotes clumping (clustering) of S. aureus (e.g., Xen29): (d) promotes cell-independent lysis of S. aureus; (e) decreases binary fission of S. aureus; and (f) binds to a polypeptide consisting of the amino acid sequence of SEQ ID NO:72 with a KD of ≤1 nM and ≥1 pM (e.g., as determined by BIACORE® analysis). In some embodiments, the Gmd-binding antibody has 2, 3, or 4 of the above characteristics. In some embodiments, the Gmd-binding antibody has at least 2, at least 3, or at least 4, of the above characteristics. In some embodiments, the Gmd-binding antibody inhibits in vitro growth of S. aureus by 5% to 100%, 10% to 80%, or 10% to 60.

In one embodiment, the disclosure provides for the treatment of an infection or infection-associated condition that comprises administering a Gmd-binding antibody (e.g., a full length antibody or a Gmd-binding antibody fragment) to a subject that has a infection or condition, or is at risk of having an infection or condition, that is associated with Gmd or with a microorganism that expresses Gmd such as, S. aureus. In another embodiment, the treatment includes the administration of a Gmd-binding antibody to an isolated tissue or cells from a subject, where the subject has an infection or condition or is at risk of having an infection or condition associated with Gmd or a microorganism that expresses Gmd, such as S. aureus.

The disclosure provides pharmaceutical compositions comprising a Gmd-binding antibody (e.g., a full length antibody or a Gmd-binding antibody fragment) and a pharmaceutically acceptable carrier. Also provided are methods for treating and/or ameliorating conditions associated with a Gmd-mediated activity or the presence of an organism that expresses Gmd (e.g., S. aureus) in a subject, comprising administering to a subject in need thereof an effective amount of a pharmaceutical composition comprising a Gmd-binding antibody provided herein. In some embodiments, the Gmd-binding antibody is administered alone. In other embodiments, the Gmd-binding antibody is administered as a combination therapy. Also provided are methods of reducing Gmd activity or the presence of an organism that expresses Gmd (e.g., S. aureus) in a subject comprising administering an effective amount of a Gmd-binding antibody to a subject in need thereof.

As provided herein, an effective amount of a Gmd-binding antibody (e.g., a full length antibody or a Gmd-binding antibody fragment) can be administered to treat a Staphylococcus infection or associated condition. In some embodiments, the administered anti-Gmd-antibody cross-blocks or competes for binding Gmd with an antibody having a VH and a VL pair disclosed in Table 1. In some embodiments, the administered anti-Gmd-antibody binds to the same epitope of Gmd as an antibody having a VH and a VL pair disclosed in Table 1. In some instances, the subject has or is at risk of having a Staphylococcus (e.g., S. aureus) infection.

In another aspect, the disclosure provides a method for treating a Staphylococcus infection that comprises administering to a subject having or at risk of having a Staphylococcus infection an effective amount of a Gmd-binding antibody (e.g., a full length antibody and a Gmd-binding antibody fragment). In some embodiments, the administration of the Gmd-binding antibody is repeated. The initial and repeated administrations can be concurrent with or in sequence relative to other therapies and carried out systemically or carried out directly to a site of the Staphylococcus infection, or both. In some embodiments, the method for treating Staphylococcus infection is used to treat Staphylococcus infection at a site that includes, but is not limited to infection of the skin, muscle, cardiac, respiratory tract, gastrointestinal tract, eye, kidney and urinary tract, and bone or joint infections. The methods of treatment as disclosed herein can be used to treat any patient in need, including humans and non-human mammals, however, the methods are particularly useful for immuno-compromised patients of any age, as well as patients that are older than 50 years of age.

In another embodiment, the disclosure provides a method for treating a S. aureus infection that comprises administering to a subject having or at risk of having a S. aureus infection an effective amount of a Gmd-binding antibody (e.g., a full length antibody and a Gmd-binding antibody fragment). In some embodiments, the administration of the Gmd-binding antibody is repeated. The initial and repeated administrations can be concurrent with or in sequence relative to other therapies and carried out systemically or carried out directly to a site of the Staphylococcus infection, or both. In some embodiments, the method is used to treat an Staphylococcus infection at a site selected from the skin, muscle, cardiac, respiratory tract, gastrointestinal tract, eye, kidney and urinary tract, and bone or joint.

In some embodiments, the disclosure provides a method for treating endocarditis that comprises administering to a subject having or at risk of having endocarditis an effective amount of a Gmd-binding antibody (e.g., a full length antibody and a Gmd-binding antibody fragment) disclosed herein. In further embodiments, the subject has or is at risk of having S. aureus associated endocarditis.

In some embodiments, the disclosure provides a method for treating osteomyelitis that comprises administering to a subject having or at risk of having a osteomyelitis an effective amount of a Gmd-binding antibody (e.g., a full length antibody and a Gmd-binding antibody fragment) disclosed herein. In some embodiments, the Gmd-binding antibody is administered to a subject having or at risk of having a bone or joint infection. In some embodiments, the subject has or is at risk of having a Staphylococcus bone or joint infection. In further embodiments, the subject has or is at risk of having a S. aureus bone or joint infection. Administration of these agents or compositions can be carried out using any of the routes described supra or otherwise known in the art; however, in particular embodiments, the agent is administered directly to the site of the bone or joint infection.

In some embodiments, the disclosure provides a method for treating device/implant associated infection that comprises administering to a subject having or at risk of having a device/implant associated infection, an effective amount of a Gmd-binding antibody (e.g., a full length antibody and a Gmd-binding antibody fragment) disclosed herein. In further embodiments, the subject has or is at risk of having S. aureus associated device/implant associated infection. In some embodiments, the implant is an orthopedic or dental implant, such as implants that replace bone or provide fixation to bone, replace articulating surfaces of a joint, provide abutment for a prosthetic, or combinations thereof. In other embodiments, the implant is for car, nose, and/or throat (“ENT”) applications. In some embodiments, the implant is a prosthesis. In other embodiments, the implant is a cardiovascular device (e.g., a cardiac valve, an alloplastic vessel wall support, or a total artificial heart implant). In other embodiments, the implant is a member selected from the group consisting of Kirschner wire, bone plates, screws, pins, tacs, rods, nails, nuts, bolts, washers, spikes, buttons, wires, fracture plates, endo- and exoprostheses, intraosseous transcutaneous prostheses, spacers, mesh, implant abutments, anchors, barbs, clamps, suture, tubes of any geometry, and combinations thereof. In additional embodiments, the implant is an ophthalmological implant. In other embodiments, the implant is not an ophthalmological implant.

In particular embodiments, the provided methods reduce the rate of infection, the severity of infection, the duration of infection, the wound healing time in the presence of infection or other complications (e.g., open contaminated wounds and infections or closed wounds at risk for infection), or any combination thereof. In certain embodiments, the preventative or therapeutic methods of treatment can reduce or altogether eliminate the total number of SRCs or abscesses, and/or increase the number of sterile SRCs or abscesses (assuming SRCs or abscesses are present). In certain embodiments, partial or complete healing of an osteolytic lesion is contemplated, as indicated by a reduction in lesion size or volume.

Surgical Site Infections (SSIs) involving medical devices (e.g., orthopedic implants) are a well-known, widespread and severe problem leading to significant patient morbidity and mortality. Medical devices often serve as a nidus for bacterial colonization and biofilms that trigger the formation of fibrous tissue around infected devices instead of bone. This scenario further complicates patient outcomes by degrading bone and decreasing the device fixation required to stabilize the segment (which is often the primary objective of the original surgery). Yet, the need to maintain the stability of the implant-bone interface makes leaving the device in place and attempting to treat the infection with, e.g., irrigation, debridement(s) and/or antibiotics the standard of care for many procedures, such as common spinal fusions. In some embodiments, the disclosure provides a method for treating SSI associated infection that comprises administering to a subject having or at risk of having a SSI, an effective amount of a Gmd-binding antibody (e.g., a full length antibody and a Gmd-binding antibody fragment) disclosed herein.

The treatment methods provided according to the disclosure can be used to treat any patient in need, however, the methods are particularly useful for immuno-compromised patients of any age, as well as patients that are older than 50 years of age.

In one embodiment, the method of introducing an orthopedic implant, medical device or graft includes administering to the patient in need of the orthopedic implant, medical device or graft an effective amount of a Gmd-binding antibody or Gmd-binding antibody fragment or a pharmaceutical composition containing the same, systemically or directly to the site of implantation. Alternatively, or in addition, the orthopedic implant, medical device or graft can be coated or treated with the monoclonal antibody or binding fragment or a pharmaceutical composition containing the same before, during, or immediately after implantation thereof at the implant site.

The orthopedic implant can be any type of implant that is susceptible to Staphylococcus infection, such as a joint prosthesis, graft or synthetic implant. Exemplary joint prostheses includes, without limitation, a knee prosthesis, hip prosthesis, finger prosthesis, elbow prosthesis, shoulder prosthesis, temperomandibular prosthesis, and ankle prosthesis. Other prosthetics can also be used. Exemplary grafts or synthetic implants include, without limitation, a vascular graft, a heart valve implant, an artificial intervertebral disk, meniscal implant, or a synthetic or allograft anterior cruciate ligament, medial collateral ligament, lateral collateral ligament, posterior cruciate ligament, Achilles tendon, and rotator cuff. Other grafts or implants can also be used.

The medical device can be any medical device that is susceptible to Staphylococcus infection. Exemplary medical devices include, without limitation, a cardiac pacemaker, cerebrospinal fluid shunt, dialysis catheter, or prosthetic heart valve.

In a further embodiment, the disclosure provides a method of introducing an orthopedic implant, tissue graft or medical device into a patient that includes administering to a patient in need of such an implant an effective amount of a monoclonal antibody, binding portion, or pharmaceutical composition disclosed herein, and introducing the orthopedic implant or medical device into the patient.

In one embodiment, the disclosure provides a method of introducing an orthopedic implant or medical device to a subject. The method includes administering to the subject an effective amount of a Gmd-binding antibody (e.g., a full length antibody and a Gmd-binding antibody fragment). In some embodiments, the administered Gmd-binding antibody (e.g., a full length antibody or a Gmd-binding antibody fragment) cross-blocks or competes for binding Gmd with an antibody having a VH and a VL pair disclosed in Table 1. In some embodiments, the administered Gmd-binding antibody binds to the same epitope of Gmd as an antibody having a VH and a VL pair disclosed in Table 1.

In another embodiment, the disclosure provides a method of introducing an orthopedic implant into a subject that comprises administering to a subject in need of an orthopedic implant an effective amount of a Gmd-binding antibody or pharmaceutical composition of the disclosure, and introducing the orthopedic implant into the subject. In one embodiment, the method comprises administering the Gmd-binding antibody or pharmaceutical composition directly to the site of implantation. In additional embodiments, the orthopedic implant is coated or treated with the Gmd-binding antibody or pharmaceutical composition before, during, or immediately after the implantation of the implant at the implant site. In some embodiments, the orthopedic implant is a joint prosthesis, graft or a synthetic implant. Exemplary joint prosthetics include, without limitation, a knee prosthetic, hip prosthetic, finger prosthetic, elbow prosthetic, shoulder prosthetic, temperomandibular prosthetic, and ankle prosthetic. Other prosthetics described herein or otherwise known in the art can also be used. Exemplary grafts or synthetic implants include, without limitation, an artificial intervertebral disk, meniscal implant, or a synthetic or allograft anterior cruciate ligament, medial collateral ligament, lateral collateral ligament, posterior cruciate ligament, Achilles tendon, and rotator cuff. Other grafts or implants are described herein or otherwise known in the art and can also be used.

In some embodiment, the disclosure provides a method of introducing an orthopedic implant into a subject wherein the method encompass the process of installing a revision total joint replacement. Where infection, particularly Staph infection of an original joint replacement occurs, the only viable treatment is a revision total joint replacement. The treatment involves the removal of the infected joint prosthesis and subsequent treatment of the subject for the underlying infection. This treatment typically takes place over an extended period of time (e.g., 6 months), during which time the subject is immobile (or has limited mobility) and receives high doses of antibiotics to treat the underlying infection. Upon treatment of the underlying infection, the new joint prosthesis is installed. In some embodiments of the present disclosure, the Gmd-binding antibodies or pharmaceutical compositions provided herein are administered to the subject after removal; of the infected joint prosthesis. In additional embodiments provided herein, immediately prior (i.e., within the two weeks preceding new joint prosthesis installation) and optionally subsequent to the installation of the new joint prosthesis, the subject is administered a Gmd-binding antibody or pharmaceutical composition disclosed herein. This treatment can be repeated one or more times during the post-installation period. Antibiotic treatment may be administered in combination with or concurrently with the Gmd-binding antibodies or pharmaceutical compositions. These treatments are effective to prevent infection or reinfection during the revision total joint replacement.

A critical step in the pathogenesis of device infections is bacterial adherence to the foreign body surface and the formation of a bacterial biofilm. A biofilm is defined as a structured community of bacterial cells enclosed in a self-produced polymeric matrix and adherent to an inert or living surface. In orthopedic trauma for example, some 5-10% of orthopedic hardware facilitates host infection with increasing incidences for open fractures, combat related injuries, and revision joint replacements. Biofilms can be formed by a single bacterial strain, although most natural biofilms are formed by multiple bacterial species (Yang et al., Int. J. Oral Sci., 3:74-81 (2011)). Applications of antibiotics are often ineffective for biofilm populations due to their unique physiology and physical matrix barrier.

In some embodiments, Gmd-binding antibody (e.g., a full length antibody or a Gmd-binding antibody fragment) provided herein is used to prevent or reduce the formation of biofilm in, for example, the context of a surgical implant, a stent, a catheter, and another indwelling medical device.

In some embodiments, the disclosure provides a method for treating a biofilm infection which comprises contacting the biofilm with a Gmd-binding antibody (e.g., a full length antibody and a Gmd-binding antibody fragment) provided herein.

In some embodiments, the disclosure provides a method for treating a biofilm infection which comprises administering an effective amount of a Gmd-binding antibody (e.g., a full length antibody or a Gmd-binding antibody fragment) to a subject having or at risk of having a biofilm infection. In some embodiments, the biofilm comprises antibiotic resistant bacteria. In further embodiments, the biofilm bacteria is methicillin-resistant S. aureus (MRSA), vancomycin resistant S. aureus (VRSA), daptomycin-resistant S. aureus (DRSA), or linezolid-resistant S. aureus (LRSA). In some embodiments, the bacteria have altered antibiotic sensitivity. In further embodiments, the bacteria have vancomycin intermediate-sensitivity S. aureus (VISA).

In some embodiments, the disclosure provides a method for treating or disrupting a biofilm and/or biofilm associated infection that comprises Gram-positive bacteria, (e.g., Staphylococcus, such as S. aureus), said method comprises contacting the biofilm with an amount of a Gmd-binding antibody (e.g., a full length antibody or a Gmd-binding antibody fragment) effective to disperse or kill gram-positive bacteria in the biofilm, including Staphylococcus (e.g., S. aureus). In some embodiments, the methods, the contacting step is performed in vitro or ex vivo so as to sterilize or decontaminate a solution, material or device, particularly intended for use by or in a human. In some embodiments, bacteria associated with the biofilm are antibiotic resistant bacteria. In further embodiments, bacteria associated with the biofilm are methicillin-resistant S. aureus (MRSA), vancomycin resistant S. aureus (VRSA), daptomycin-resistant S. aureus (DRSA), or linezolid-resistant S. aureus (LRSA).

In additional embodiments, the disclosure provides a method of killing gram-positive bacteria in a biofilm, that comprises the step of contacting the biofilm with an amount of a provided Gmd-binding antibody (e.g., a full length antibody or a Gmd-binding antibody fragment) that is effective to kill gram-positive bacteria in the biofilm (e.g., S. aureus), whereby the number of gram-positive bacteria in the human is reduced. In a particular aspect of this method, wherein the subject is exposed to or at risk of being exposed to Staphylococcus (such as S. aureus) bacteria. In one embodiment, method is performed in vivo. In one embodiment, method is performed in vitro or ex vivo. In one embodiment, method is performed ex vivo. In one embodiment, method is performed in vitro or ex vivo so as to sterilize or decontaminate a solution, material or device, particularly intended for use by or in a mammalian subject (e.g., a human).

In an additional embodiment, the disclosure provides a method for preventing, dispersing or treating a gram-positive bacterial biofilm in a subject (e.g., human), wherein the biofilm comprises one or more Staphylococcus or Streptococcus bacteria and wherein the method comprises the step of administering to a subject having, or at risk of having a gram-positive bacterial biofilm, an amount of Gmd-binding antibody (e.g., a full length antibody or a Gmd-binding antibody fragment) effective to prevent, disperse or treat the biofilm. In further embodiments, the number of gram-positive bacteria in the subject is reduced. In particular embodiments, the gram positive bacteria comprises S. aureus bacteria. In further embodiments, the gram-positive bacteria comprises MRSA.

In some embodiments, the disclosure provides a method for reducing the population of gram-positive bacteria in a biofilm that comprises the step of contacting the biofilm with an amount of a Gmd-binding antibody (e.g., a full length antibody or a Gmd-binding antibody fragment) effective to kill or release at least a portion of the gram-positive bacteria in the biofilm. In further embodiments, the disclosure provides a method for dispersing or treating an antibiotic-resistant S. aureus infection which involves or includes a biofilm in a human that comprises the step of administering to a human with an antibiotic-resistant S. aureus biofilm infection, an amount of a Gmd-binding antibody (e.g., a full length antibody and a Gmd-binding antibody fragment) and effective to disperse the biofilm and kill S. aureus therein and/or released therefrom, whereby the number of S. aureus in the human is reduced and/or the biofilm and attendant infection is reduced. In some embodiments, the bacteria or biofilm bacteria comprises an antibiotic resistant bacteria. In further embodiments, the bacteria comprises an antibiotic resistant bacteria selected from the group consisting of methicillin-resistant S. aureus (MRSA), vancomycin resistant S. aureus (VRSA), daptomycin-resistant S. aureus (DRSA), and linezolid-resistant S. aureus (LRSA).

In some embodiments, the disclosure provides a method of dispersing gram-positive bacteria in a biofilm that comprises the step of contacting the biofilm with an amount of Gmd-binding antibody (e.g., a full length antibody or a Gmd-binding antibody fragment) effective to disperse gram-positive bacteria in a biofilm, including S. aureus. In one embodiment, the methods are performed in vitro or ex vivo so as to sterilize or decontaminate a solution, material or device, particularly intended for use by or in a human. The bacteria may be antibiotic resistant, including methicillin-resistant S. aureus (MRSA), vancomycin resistant S. aureus (VRSA), daptomycin-resistant S. aureus (DRSA), or linezolid-resistant S. aureus (LRSA). The bacteria may have altered antibiotic sensitivity, such as for example, vancomycin intermediate-sensitivity S. aureus (VISA).

In another embodiment, the disclosure provides a method for inhibiting or reducing bacterial biofilm formation on a surface that comprises comprising the step of applying to the surface an amount of a Gmd-binding antibody (e.g., a full length antibody or a Gmd-binding antibody fragment). Coating biomedical materials with a Gmd-binding antibody (e.g., a full length antibody or a Gmd-binding antibody fragment) may also prove successful in preventing early adherence of bacteria, including staphylococci, to the implants, thus averting biofilm formation. The disclosure thus also provides a method for reducing or preventing biofilm growth on the surface of a device or implant that comprises administering or coating the device or implant with a Gmd-binding antibody disclosed herein. In some embodiments, the device or implant is an orthopedic or dental implant, such as a implant that replace bone or provide fixation to bone, replace articulating surfaces of a joint, provide abutment for a prosthetic, or combinations thereof. In another embodiment, the device or implant is for car, nose, and/or throat (“ENT”) applications (e.g., car tubes, endotracheal tubes, ventilation tubes, cochlear implants and bone anchored hearing devices). In yet another embodiment, the device or implant is a cardiovascular device such as a cardiac valve, an alloplastic vessel wall support, or a total artificial heart implant. In some embodiments, the device or implant is an ophthalmological implant. In other embodiments, the device or implant is not an ophthalmological implant.

In additional embodiments, the disclosure provides methods that comprise administering a therapeutically effective amount of a Gmd-binding antibody (e.g., a full length antibody and a Gmd-binding antibody fragment), alone or in combination with one or more additional therapies (e.g., one or more additional antimicrobials or other therapeutic agents) to a subject having, or at risk for having a Streptococcus (e.g., S. aureus) infection and/or associated-condition such as, fever, bacteremia, endocarditis, osteomyelitis, pneumonia, sepsis, dermonecrosis, and mastitis; and disorders/conditions associated with one or more of the above diseases or conditions.

The disclosure also provides the use of a Gmd-binding antibody (e.g., a full length antibody or a Gmd-binding antibody fragment) provided herein for diagnostic monitoring of protein levels (e.g., Gmd levels) in blood or tissue as part of a clinical testing procedure, e.g., to determine the efficacy of a given treatment regimen. For example, detection can be facilitated by coupling a Gmd-binding antibody to a detectable substance. Examples of detectable substances include various enzymes, prosthetic groups, fluorescent materials, luminescent materials, bioluminescent materials, and/or radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin; and examples of suitable radioactive material include ¹²⁵I, ¹³¹I, ³⁵S, or ³H.

### Pharmaceutical Compositions and Administration Methods

Methods of preparing and administering a Gmd-binding antibody (e.g., a full length antibody or a Gmd-binding antibody fragment) to a subject in need thereof are known to or are readily determined by those of ordinary skill in the art. The route of administration of the Gmd-binding antibodies can be, for example, oral, parenteral, by inhalation or topical. The term parenteral includes, e.g., intravenous, intraarterial, intraperitoneal, intramuscular, intraocular, subcutaneous, rectal, or vaginal administration. While all these forms of administration are clearly contemplated as being within the scope of the disclosure, another example of a form for administration would be a solution for injection, in particular for intravenous or intraarterial injection or drip. Usually, a suitable pharmaceutical composition can comprise a buffer (e.g., acetate, phosphate or citrate buffer), a surfactant (e.g., polysorbate), optionally a stabilizer agent (e.g., human albumin), etc. In other methods compatible with the teachings herein, Gmd-binding antibodies as provided herein can be delivered directly to the organ and/or site of a fibrosis or tumor, thereby increasing the exposure of the diseased tissue to therapeutic agent. In one embodiment, the administration is directly to the airway, e.g., by inhalation or intranasal administration.

As discussed herein, Gmd-binding antibodies (e.g., full length antibodies and Gmd-binding antibody fragments) can be administered in a pharmaceutically effective amount for the in vivo treatment of Streptococcus (e.g., S. aureus) or Gmd-mediated diseases and conditions such as, S. aureus infection, fever, bacteremia, endocarditis, osteomyelitis, pneumonia, sepsis, dermonecrosis, and mastitis. In this regard, it will be appreciated that the disclosed Gmd-binding antibodies can be formulated so as to facilitate administration and promote stability of the active agent. Pharmaceutical compositions in accordance with the disclosure can comprise a pharmaceutically acceptable, non-toxic, sterile carrier such as physiological saline, non-toxic buffers, preservatives and the like. For the purposes of the instant application, a pharmaceutically effective amount of a Gmd-binding antibody, conjugated or unconjugated, means an amount sufficient to achieve effective binding to Gmd and to achieve a benefit, e.g., to ameliorate symptoms of a infection or condition or to detect a substance or a cell. Suitable formulations for use in therapeutic methods disclosed herein are described in Remington's Pharmaceutical Sciences (Mack Publishing Co.) 16th ed. (1980).

Certain pharmaceutical compositions provided herein can be orally administered in an acceptable dosage form including, e.g., capsules, tablets, aqueous suspensions or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of a Gmd-binding antibody (e.g., a full length antibody and Gmd-binding antibody fragment) that can be combined with carrier materials to produce a single dosage form will vary depending upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

Gmd-binding antibodies (e.g., full length antibodies and Gmd-binding antibody fragments) provided herein can be administered to a human or other subject in accordance with the aforementioned methods of treatment in an amount sufficient to produce a therapeutic effect. The Gmd-binding antibodies (can be administered to such human or other subject in a conventional dosage form prepared by combining the Gmd-binding antibodies with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables. A cocktail comprising one or more different Gmd-binding antibodies can also be used.

Therapeutically effective doses of Gmd-binding compositions for treatment of a Streptococcus (e.g., S. aureus) infection or condition (e.g., infection, fever, bacteremia, endocarditis, osteomyelitis, pneumonia, sepsis, dermonecrosis, and mastitis) vary depending upon many different factors, including means of administration, target site, physiological state of the subject, whether the subject is human or an animal, other medications administered, and whether treatment is prophylactic or therapeutic. Usually, the subject is a human, but non-human mammals including transgenic mammals can also be treated. Treatment dosages can be titrated using routine methods known to those of ordinary skill in the art to optimize safety and efficacy.

Effective doses of the provided Gmd-binding antibodies and pharmaceutical compositions for the treatment of bacterial infections and their associated conditions will vary depending upon many different factors, including mode of administration, target site, physiological state of the patient, other medications administered, and whether treatment is prophylactic or therapeutic. In prophylactic applications, a relatively low dosage is administered at relatively infrequent intervals over a long period of time. Some subjects can potentially continue to receive treatment for the rest of their lives. In therapeutic applications, a relatively high dosage at relatively short intervals is sometimes required until progression of the disease is reduced or terminated, and preferably until the subject shows partial or complete amelioration of symptoms of infection or an associated condition. Thereafter, the subject can be administered a prophylactic regime. For prophylactic treatment against Staphylococcus infection, it is intended that the pharmaceutical composition(s) can be administered prior to exposure of an individual to the Staphylococcus band that the resulting immune response can inhibit or reduce the severity of the bacterial infection such that the bacteria can be eliminated from the subject. For example, the Gmd-binding antibody or the pharmaceutical composition can be administered prior to, during, and/or immediately following a surgical procedure, such as joint replacement or any surgery involving a prosthetic implant. In additional embodiments, a Gmd-binding antibody is administered to a subject before, during, and/or after a surgical excision/removal procedure.

For passive immunization with a Gmd-binding antibody or Gmd-binding antibody fragment of the disclosure, the dosage ranges from about 0.0001 to about 100 mg/kg, and more usually about 0.01 to about 5 mg/kg, of the host body weight. For example, dosages can be about 1 mg/kg body weight or about 10 mg/kg body weight, or within the range of about 1 to about 10 mg/kg. An exemplary treatment regime entails administration once per every two weeks or once a month or once every 3 to 6 months. In some methods, two or more monoclonal antibodies with different binding specificities are administered simultaneously, in which case the dosage of each antibody administered falls within the ranges indicated. Passive immunization antibody therapy is typically administered on multiple occasions. Intervals between single dosages can be daily, weekly, monthly, or yearly. In some methods, dosage is adjusted to achieve a plasma antibody concentration of 1-1000 μg/ml and in some methods 25-300 g/ml, alternatively, antibody can be administered as a sustained release formulation, in which case less frequent administration is required. Dosage and frequency vary depending on the half-life of the Gmd-binding antibody in the subject.

To ameliorate the symptoms of a particular infection or condition by administration of a Gmd-binding antibody (e.g., a full length antibody or a Gmd-binding antibody fragment) refers to any lessening, whether permanent or temporary, lasting or transient that can be attributed to or associated with administration of the Gmd-binding.

The disclosure also provides for the use of a Gmd-binding antibody, such as, a Gmd-binding antibody in the manufacture of a medicament for example, for treating, preventing or ameliorating a Staphylococcus (e.g., S. aureus) infection or a Staphylococcus (e.g., S. aureus) infection associated condition (e.g., fever, bacteremia, endocarditis, osteomyelitis, pneumonia, sepsis, dermonecrosis, or mastitis).

### Combination Therapies

In some embodiments, a Gmd-binding antibody (e.g., a full length antibody or a Gmd-binding antibody fragment) is administered in combination with one or more other therapies. Such therapies include additional therapeutic agents as well as other medical interventions. Exemplary therapies include, for example, a surgical procedure, such as joint replacement or any surgery involving a prosthetic implant.

The complementary agent may be an antibiotic, such as erythromycin, clarithromycin, azithromycin, roxithromycin, other members of the macrolide family, penicillins, cephalosporins, and any combinations thereof in amounts which are effective to synergistically enhance therapeutic effect of the lytic enzyme. Virtually any other antibiotic may be used with the altered and/or unaltered lytic enzyme. Antibiotics affecting cell wall peptidoglycan biosynthesis include: glycopeptides, which inhibit peptidoglycan synthesis by preventing the incorporation of N-acetylmuramic acid (NAM) and N-acetylglucosamine (NAG) peptide subunits into the peptidoglycan matrix. Available glycopeptides include vancomycin and teicoplanin; Penicillins, which act by inhibiting the formation of peptidoglycan cross-links. The functional group of penicillins, the beta-lactam moiety, binds and inhibits DD-transpeptidase that links the peptidoglycan molecules in bacteria. Hydrolytic enzymes continue to break down the cell wall, causing cytolysis or death due to osmotic pressure. Common penicillins include oxacillin, ampicillin and cloxacillin; and Polypeptides, which interfere with the dephosphorylation of the C₅₅-isoprenyl pyrophosphate, a molecule that carries peptidoglycan building-blocks outside of the plasma membrane. A cell wall-impacting polypeptide is bacitracin. Other useful and relevant antibiotics include vancomycin, linezolid, and daptomycin.

The disclosed Gmd-binding antibodies and pharmaceutical compositions can be administered alone or in combination with one or more additional therapeutic agents. In some embodiments, the Gmd-binding antibody (e.g., a full-length Gmd-antibody or a Gmd-binding antibody fragment) is administered in combination with one or more antimicrobial agents. In some embodiments, a Gmd-binding antibody is administered in combination with an antibiotic agent or immunotherapeutic agent. Exemplary antibiotic agents that can be administered with the provided antibodies and pharmaceutical compositions include, without limitation, vancomycin, tobramycin, cefazolin, erythromycin, clindamycin, rifampin, gentamycin, fusidic acid, minocycline, co-trimoxazole, clindamycin, linezolid, quinupristin-dalfopristin, daptomycin, tigecycline, dalbavancin, telavancin, oritavancin, ceftobiprole, ceftaroline, iclaprim, the carbapenem CS-023/RO-4908463, and combinations thereof. Exemplary immunotherapeutic agents that can be administered with the provided antibodies and pharmaceutical compositions include, without limitation, tefibazumab, BSYX-A1 10, Aurexis™, and combinations thereof. The above lists of antibiotic agents and immunotherapeutic agents are intended to be non-limiting examples; thus, other antibiotic agents or immunotherapeutic agents are also contemplated. Combinations or mixtures of the second therapeutic agent can also be used for the purposes of the disclosure. These agents can be administered contemporaneously or as a single formulation.

In further embodiments, the Gmd-binding antibody is administered in combination a member selected from the group consisting of: (a) a biguanide (e.g., buformin, metformin, or phenformin), (b) insulin, (c) somatostatin, (d) an alpha-glucosidase inhibitor (e.g., voglibose, miglitol, or acarbose), (e) a DPP-IV inhibitor (e.g., sitagliptin, vildagliptin, alogliptin, or saxagliptin (for example, as disclosed in U.S. Pat. No. 6,699,871B1)), (f) an LXR modulator, (g) an insulin secretagogue (e.g., acetohexamide, carbutamide, chlorpropamide, glibornuride, gliclazide, glimerpiride, glipizide, gliquidine, glisoxepid, glyburide, glyhexamide, glypinamide, phenbutamide, tolazamide, tolbutamide, tolcyclamide, nateglinide or repaglinide), (k) a CB1 inhibitor (e.g., rimonabant, taranabant, or other therapeutic compounds disclosed in Intl. Appl. Publ. Nos. WO03/077847A2 and WO05/000809A1, or (l) sibutramine, (m) topiramate, (n) orlistat, (o) Qnexa, (p) mevastatin, (q) simvastatin, (r) ezetimibe, (s) atorvastatin, (t) naltrexone, (u) bupriopion, (v) phentermine, (w) hydrochlorothiazide, or (x) losartan.

### Kits Comprising Gmd-Binding Antibodies

This disclosure further provides kits that include a Gmd-binding antibody (e.g., a full length antibody or a Gmd-binding antibody fragment) in suitable packaging, and written material and that can be used to perform the methods described herein. The written material can include any of the following information: instructions for use, discussion of clinical studies, listing of side effects, scientific literature references, package insert materials, clinical trial results, and/or summaries of these and the like. The written material can indicate or establish the activities and/or advantages of the composition, and/or describe dosing, administration, side effects, drug interactions, or other information useful to the health care provider. Such information can be based on the results of various studies, for example, studies using experimental animals involving in vivo models and/or studies based on human clinical trials. The kit can further contain another therapy (e.g., another agent) and/or written material such as that described above that serves to provide information regarding the other therapy (e.g., the other agent).

In certain embodiments, a kit comprises at least one purified Gmd-binding antibody (e.g., a full length antibody or a Gmd-binding antibody fragment) in one or more containers. In some embodiments, the kits contain all of the components necessary and/or sufficient to perform a detection assay, including all controls, directions for performing assays, and/or any necessary software for analysis and presentation of results.

### Immunoassays

Gmd-binding antibodies (e.g., full length antibodies and Gmd-binding antibody fragments) can be assayed for immunospecific binding by any method known in the art. The immunoassays that can be used include, but are not limited to, competitive and non-competitive assay systems using techniques such as Western blots, radioimmunoassays (REA), ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, or protein A immunoassays. Such assays are routine and known in the art (see, e.g., Ausubel et al., eds, (1994) Current Protocols in Molecular Biology (John Wiley & Sons, Inc., NY) Vol. 1, which is herein incorporated by reference in its entirety).

Gmd-binding antibodies (e.g., full length antibodies and Gmd-binding antibody fragments) provided herein can be employed histologically, as in immunofluorescence, immunoelectron microscopy or non-immunological assays, for in situ detection of Gmd or conserved variants or peptide fragments thereof. In situ detection can be accomplished according to methods known in the art. Those of ordinary skill in the art will be able to determine operative and optimal assay conditions for each determination by employing routine experimentation. Methods suitable for determination of binding characteristics of a Gmd-binding antibody are described herein or otherwise known in the art. Equipment and software designed for such kinetic analyses are commercially available (e.g., BIACORE®, BIAevaluation® software, GE Healthcare; KINEXA® Software, Sapidyne Instruments).

Unless otherwise indicated, the practice of the disclosure employs conventional techniques of cell biology, cell culture, molecular biology, transgenic biology, microbiology, recombinant DNA, and immunology, which are within the skill of the art.

The following examples are offered by way of illustration and not by way of limitation.

The foregoing description of the specific embodiments, will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

The disclosure of U.S. Appl. No. 63/149,003, filed Feb. 12, 2021 is herein incorporated by reference in its entirety.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

### EXAMPLES

The examples below are intended to exemplify the practicing the claimed subject matter, but are by no means intended to limit the scope thereof.

### Example 1. Generation of Humanized Gmd-Binding Antibodies

A multi-round selection procedure was used to select for humanized human IgG antibodies that bind Gmd with high affinity.

### Example 2. Characterization of Gmd-Binding Antibodies

Exemplary optimized humanized anti-Gmd-binding antibodies generated according to the previous example were further characterized by sequence, SPR, and cell-based lipolysis inhibition assay analyses.

Sequences of exemplary humanized Gmd-binding antibodies generated according to the methods described in Example 1 are presented in Table 1 (exemplary CDR sequences are underscored). Sequences corresponding to the parental 1C11 antibody are presented in Table 2.

TABLE 1
Exemplary Humanized Antibodies
X4

| | |
|---|---|
| VH | QVQLVQSGAEVKKPGASVKVSCKASGYTFTTYGMSWIRQSPSRGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 1) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WIRQSPSRGLEWMG (SEQ ID NO: 17) |
| VH CDR2 | GIIPIFGTASYAQKFQG (SEQ ID NO: 11) |
| VH FR3 | FVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGATCAGGCAGTCCCCATCGAGAGGCCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 24) |
| VL | AIQLTQSPSSLSASVGDRVTITCRASQSISNNLHWYQQKPGQAPRLLIYYASRSISGVPSRFSGSGSGTD |
| Protein | FTFTISSLQPEDIATYYCQQSNSWPLTFGQGTKVEIK (SEQ ID NO: 34) |
| VL FR1 | AIQLTQSPSSLSASVGDRVTITC (SEQ ID NO: 46) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GVPSRFSGSGSGTDFTFTISSLQPEDIATYYC (SEQ ID NO: 54) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGGTCCCATCAAGGTTCAGTGGAAGTGGATCTGGGACAGAT |
| | TTTACTTTCACCATCAGCAGCCTGCAGCCTGAAGATATTGCAACATATTACTGTCAACAGAGTAACAGCT |
| | GGCCGCTCACGTTCGGCCAAGGGACCAAGGTGGAAATCAAA (SEQ ID NO: 63) |

X13

| | |
|---|---|
| VH | QVQLVQSGAEVKKPGASVKVSCKASGYTFTTYGMSWIRQSPSRGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 1) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WIRQSPSRGLEWMG (SEQ ID NO: 17) |
| VH CDR2 | GIIPIFGTASYAQKFQG (SEQ ID NO: 11) |
| VH FR3 | FVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGATCAGGCAGTCCCCATCGAGAGGCCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 24) |
| VL | AIQLTQSPSSLSASVGDRVTITCRASQSISNNLHWYQQKPGKAPKLLIYYASRSISGVPSRFSGSGSGTE |
| Protein | FTLTISSLQPDDFATYYCQQSNSWPLTFGQGTKVEIK (SEQ ID NO: 35) |
| VL FR1 | AIQLTQSPSSLSASVGDRVTITC (SEQ ID NO: 45) |
| VL CDR1 | RASQSISNNLH (SEQ ID NO: 42) |
| VL FR2 | WYQQKPGKAPKLLIY (SEQ ID NO: 49) |
| VL CDR2 | YASRSIS (SEQ ID NO: 43) |
| VL FR3 | GVPSRFSGSGSGTEFTLTISSLQPDDFATYYC (SEQ ID NO: 55) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTATCAGCAGAAACCAGGGAAAGCTCCTAAGCTCCT |
| | GATCTATTATGCTTCCCGGTCCATCTCTGGGGTCCCATCAAGGTTCAGCGGCAGTGGATCTGGGACAGAA |
| | TTCACTCTCACCATCAGCAGCCTGCAGCCTGATGATTTTGCAACTTATTACTGTCAACAGAGTAACAGCT |
| | GGCCGCTCACGTTCGGCCAAGGGACCAAGGTGGAAATCAAA (SEQ ID NO: 64) |

X5

| | |
|---|---|
| VH | QVQLVQSGAEVKKPGASVKVSCKASGYTFTTYGMSWIRQSPSRGLEWLGWINTYSGVPTYADDFKGRFVF |
| Protein | SLDTSVSTAYLQICSLKAEDTAVYYCAREEYSSGYAAWFPYWGQGTLVTVSS (SEQ ID NO: 2) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WIRQSPSRGLEWLG (SEQ ID NO: 16) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGATCAGGCAGTCCCCATCGAGAGGCCTTGAGTG |
| | GCTGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCAGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 25) |
| VL | AIQLTQSPSSLSASVGDRVTITCRASQSISNNLHWYQQKPGQAPRLLIYYASRSISGVPSRFSGSGSGTD |
| Protein | FTFTISSLQPEDIATYYCQQSNSWPLTFGQGTKVEIK (SEQ ID NO: 34) |
| VL FR1 | AIQLTQSPSSLSASVGDRVTITC (SEQ ID NO: 46) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GVPSRFSGSGSGTDFTFTISSLQPEDIATYYC (SEQ ID NO: 54) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGGTCCCATCAAGGTTCAGTGGAAGTGGATCTGGGACAGAT |
| | TTTACTTTCACCATCAGCAGCCTGCAGCCTGAAGATATTGCAACATATTACTGTCAACAGAGTAACAGCT |
| | GGCCGCTCACGTTCGGCCAAGGGACCAAGGTGGAAATCAAA (SEQ ID NO: 63) |

X6

| | |
|---|---|
| VH | QVQLVQSGAEVKKPGASVKVSCKASGYTFTTYGMSWIRQSPSRGLEWLGWINTYSGVPTYADDFKGRFVF |
| Protein | SLDTSVSTAYLQICSLKAEDTAVYYCAREEYSSGYAAWFPYWGQGTLVTVSS (SEQ ID NO: 2) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WIRQSPSRGLEWLG (SEQ ID NO: 16) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGATCAGGCAGTCCCCATCGAGAGGCCTTGAGTG |
| | GCTGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCAGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 25) |
| VL | EIVLTQSPATLSLSPGERATLSCRASQSISNNLHWYQQKPGQAPRLLIYYASRSISGVPSRFSGSGSGTD |
| Protein | FTFTISSLEAEDAATYYCQQSNSWPLTFGQGTKVEIK (SEQ ID NO: 37) |
| VL FR1 | EIVLTQSPATLSLSPGERATLSC (SEQ ID NO: 47) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GVPSRFSGSGSGTDFTFTISSLEAEDAATYYC (SEQ ID NO: 56) |
| VL CDR3 | QQSNSWPLT (SEQ ID NO: 44) |
| VL FR4 | FGQGTKVEIK (SEQ ID NO: 61) |
| VL DNA | GAAATTGTGTTGACACAGTCTCCAGCCACCCTGTCTTTGTCTCCAGGGGAAAGAGCCACCCTCTCCTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGGTCCCCTCGAGGTTCAGTGGCAGTGGATCTGGGACAGAT |
| | TTCACCTTTACCATCAGTAGCCTGGAAGCTGAAGATGCTGCAACATATTACTGTCAACAGAGTAACAGCT |
| | GGCCGCTCACGTTCGGCCAAGGGACCAAGGTGGAAATCAAA (SEQ ID NO: 66) |

X14

| | |
|---|---|
| VH | QVQLVQSGAEVKKPGASVKVSCKASGYTFTTYGMSWIRQSPSRGLEWLGWINTYSGVPTYADDFKGRFVF |
| Protein | SLDTSVSTAYLQICSLKAEDTAVYYCAREEYSSGYAAWFPYWGQGTLVTVSS (SEQ ID NO: 2) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WIRQSPSRGLEWLG (SEQ ID NO: 16) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGATCAGGCAGTCCCCATCGAGAGGCCTTGAGTG |
| | GCTGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCAGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 25) |
| VL | AIQLTQSPSSLSASVGDRVTITCRASQSISNNLHWYQQKPGKAPKLLIYYASRSISGVPSRFSGSGSGTE |
| Protein | FTLTISSLQPDDFATYYCQQSNSWPLTFGQGTKVEIK (SEQ ID NO: 35) |
| VL FR1 | AIQLTQSPSSLSASVGDRVTITC (SEQ ID NO: 45) |
| VL CDR1 | RASQSISNNLH (SEQ ID NO: 42) |
| VL FR2 | WYQQKPGKAPKLLIY (SEQ ID NO: 49) |
| VL CDR2 | YASRSIS (SEQ ID NO: 43) |
| VL FR3 | GVPSRFSGSGSGTEFTLTISSLQPDDFATYYC (SEQ ID NO: 55) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTATCAGCAGAAACCAGGGAAAGCTCCTAAGCTCCT |
| | GATCTATTATGCTTCCCGGTCCATCTCTGGGGTCCCATCAAGGTTCAGCGGCAGTGGATCTGGGACAGAA |
| | TTCACTCTCACCATCAGCAGCCTGCAGCCTGATGATTTTGCAACTTATTACTGTCAACAGAGTAACAGCT |
| | GGCCGCTCACGTTCGGCCAAGGGACCAAGGTGGAAATCAAA (SEQ ID NO: 64) |

X7

| | |
|---|---|
| VH | EVQLVQSGAEVKKPGESLRISCKGSGYTFTTYGMSWIRQSPSRGLEWLGWINTYSGVPTYADDFKGRFVF |
| Protein | SLDTSVSTAYLQICSLKAEDTAVYYCAREEYSSGYAAWFPYWGQGTLVTVSS (SEQ ID NO: 3) |
| VH FR1 | EVQLVQSGAEVKKPGESLRISCKGSGYTFT (SEQ ID NO: 14) |
| VH CDR1 | SYAIS (SEQ ID NO: 10) |
| VH FR2 | WIRQSPSRGLEWLG (SEQ ID NO: 16) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | GAAGTGCAGCTGGTGCAGTCTGGAGCAGAGGTGAAAAAGCCCGGGGAGTCTCTGAGGATCTCCTGTAAGG |
| | GTTCTGGGTATACCTTCACAACGTATGGAATGAGCTGGATCAGGCAGTCCCCATCGAGAGGCCTTGAGTG |
| | GCTGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 26) |
| VL | EIVLTQSPDFQSVTPKEKVTITCRASQSISNNLHWYQQKPGKAPKLLIYYASRSISGIPPRFSGSGYGTD |
| Protein | FTLTINNIESEDAAYYFCQQSNSWPLTFGQGTKVEIK (SEQ ID NO: 33) |
| VL FR1 | EIVLTQSPDFQSVTPKEKVTITC (SEQ ID NO: 45) |
| VL CDR1 | RASQSISNNLH (SEQ ID NO: 42) |
| VL FR2 | WYQQKPGKAPKLLIY (SEQ ID NO: 49) |
| VL CDR2 | YASRSIS (SEQ ID NO: 43) |
| VL FR3 | GIPPRFSGSGYGTDFTLTINNIESEDAAYYFC (SEQ ID NO: 53) |
| VL CDR3 | QQSNSWPLT (SEQ ID NO: 44) |
| VL FR4 | FGQGTKVEIK (SEQ ID NO: 61) |
| VL DNA | GAAATTGTGCTGACTCAGTCTCCAGACTTTCAGTCTGTGACTCCAAAGGAGAAAGTCACCATCACCTGCA |
| | GGGCCAGCCAAAGTATTAGCAACAACCTACACTGGTATCAGCAGAAACCAGGGAAAGCTCCTAAGCTCCT |
| | GATCTATTATGCTTCCCGGTCCATCTCTGGGATCCCACCTCGATTCAGTGGCAGCGGGTATGGAACAGAT |
| | TTTACCCTCACAATTAATAACATAGAATCTGAGGATGCTGCATATTACTTCTGTCAACAGAGTAACAGCT |
| | GGCCGCTCACGTTCGGCCAAGGGACCAAGGTGGAAATCAAA (SEQ ID NO: 62) |

X8

| | |
|---|---|
| VH | EVQLVQSGAEVKKPGESLRISCKGSGYTFTTYGMSWIRQSPSRGLEWLGWINTYSGVPTYADDFKGRFVF |
| Protein | SLDTSVSTAYLQICSLKAEDTAVYYCAREEYSSGYAAWFPYWGQGTLVTVSS (SEQ ID NO: 3) |
| VH FR1 | EVQLVQSGAEVKKPGESLRISCKGSGYTFT (SEQ ID NO: 14) |
| VH CDR1 | SYAIS (SEQ ID NO: 10) |
| VH FR2 | WIRQSPSRGLEWLG (SEQ ID NO: 16) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | GAAGTGCAGCTGGTGCAGTCTGGAGCAGAGGTGAAAAAGCCCGGGGAGTCTCTGAGGATCTCCTGTAAGG |
| | GTTCTGGGTATACCTTCACAACGTATGGAATGAGCTGGATCAGGCAGTCCCCATCGAGAGGCCTTGAGTG |
| | GCTGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 26) |
| VL | EIVLTQSPATLSLSPGERATLSCRASQSISNNLHWYLQKPGQSPQLLIYYASRSISGVPDRFSGSGSGTD |
| Protein | FTLKISRVEAEDVGVYYCQQSNSWPLTFGQGTKVEIK (SEQ ID NO: 39) |
| VL FR1 | EIVLTQSPATLSLSPGERATLSC (SEQ ID NO: 47) |
| VL CDR1 | RASQSISNNLH (SEQ ID NO: 42) |
| VL FR2 | WYLQKPGQSPQLLIY (SEQ ID NO: 51) |
| VL CDR2 | YASRSIS (SEQ ID NO: 43) |
| VL FR3 | GVPDRFSGSGSGTDFTLKISRVEAEDVGVYYC (SEQ ID NO: 58) |
| VL CDR3 | QQSNSWPLT (SEQ ID NO: 44) |
| VL FR4 | FGQGTKVEIK (SEQ ID NO: 61) |
| VL DNA | GAAATTGTGTTGACACAGTCTCCAGCCACCCTGTCTTTGTCTCCAGGGGAAAGAGCCACCCTCTCCTGCA |
| | GGGCCAGCCAAAGTATTAGCAACAACCTACACTGGTACCTGCAGAAGCCAGGGCAGTCTCCACAGCTCCT |
| | GATCTATTATGCTTCCCGGTCCATCTCIGGGGTCCCAGACAGGTTCAGTGGCAGTGGGTCAGGCACTGAT |
| | TTCACACTGAAAATCAGCAGGGTGGAGGCTGAGGATGTTGGAGTTTATTACTGTCAACAGAGTAACAGCT |
| | GGCCGCTCACGTTCGGCCAAGGGACCAAGGTGGAAATCAAA (SEQ ID NO: 68) |

X12

| | |
|---|---|
| VH | EVQLVQSGAEVKKPGESLRISCKGSGYTFTTYGMSWIRQSPSRGLEWLGWINTYSGVPTYADDFKGRFVF |
| Protein | SLDTSVSTAYLQICSLKAEDTAVYYCAREEYSSGYAAWFPYWGQGTLVTVSS (SEQ ID NO: 3) |
| VH FR1 | EVQLVQSGAEVKKPGESLRISCKGSGYTFT (SEQ ID NO: 14) |
| VH CDR1 | SYAIS (SEQ ID NO: 10) |
| VH FR2 | WIRQSPSRGLEWLG (SEQ ID NO: 16) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | GAAGTGCAGCTGGTGCAGTCTGGAGCAGAGGTGAAAAAGCCCGGGGAGTCTCTGAGGATCTCCTGTAAGG |
| | GTTCTGGGTATACCTTCACAACGTATGGAATGAGCTGGATCAGGCAGTCCCCATCGAGAGGCCTTGAGTG |
| | GCTGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 26) |
| VL | EIVLTQSPDFQSVTPKEKVTITCRASQSISNNLHWYQQKPGKAPKLLIYYASRSISGIPPRFSGSGYGTD |
| Protein | FTLTINNIESEDAAYYFCQQSNSWPLTFGQGTKVEIK (SEQ ID NO: 33) |
| VL FR1 | EIVLTQSPDFQSVTPKEKVTITC (SEQ ID NO: 44) |
| VL CDR1 | RASQSISNNLH (SEQ ID NO: 42) |
| VL FR2 | WYQQKPGKAPKLLIY (SEQ ID NO: 49) |
| VL CDR2 | YASRSIS (SEQ ID NO: 43) |
| VL FR3 | GIPPRFSGSGYGTDFTLTINNIESEDAAYYFC (SEQ ID NO: 53) |
| VL CDR3 | QQSNSWPLT (SEQ ID NO: 44) |
| VL FR4 | FGQGTKVEIK (SEQ ID NO: 61) |
| VL DNA | GAAATTGTGCTGACTCAGTCTCCAGACTTTCAGTCTGTGACTCCAAAGGAGAAAGTCACCATCACCTGCA |
| | GGGCCAGCCAAAGTATTAGCAACAACCTACACTGGTATCAGCAGAAACCAGGGAAAGCTCCTAAGCTCCT |
| | GATCTATTATGCTTCCCGGTCCATCTCTGGGATCCCACCTCGATTCAGTGGCAGCGGGTATGGAACAGAT |
| | TTTACCCTCACAATTAATAACATAGAATCTGAGGATGCTGCATATTACTTCTGTCAACAGAGTAACAGCT |
| | GGCCGCTCACGTTCGGCCAAGGGACCAAGGTGGAAATCAAA (SEQ ID NO: 62) |

X28

| | |
|---|---|
| VH | EVQLVQSGAEVKKPGESLRISCKGSGYTFTTYGMSWIRQSPSRGLEWLGWINTYSGVPTYADDFKGRFVF |
| Protein | SLDTSVSTAYLQICSLKAEDTAVYYCAREEYSSGYAAWFPYWGQGTLVTVSS (SEQ ID NO: 3) |
| VH FR1 | EVQLVQSGAEVKKPGESLRISCKGSGYTFT (SEQ ID NO: 14) |
| VH CDR1 | SYAIS (SEQ ID NO: 10) |
| VH FR2 | WIRQSPSRGLEWLG (SEQ ID NO: 16) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | GAAGTGCAGCTGGTGCAGTCTGGAGCAGAGGTGAAAAAGCCCGGGGAGTCTCTGAGGATCTCCTGTAAGG |
| | GTTCTGGGTATACCTTCACAACGTATGGAATGAGCTGGATCAGGCAGTCCCCATCGAGAGGCCTTGAGTG |
| | GCTGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 26) |
| VL | EIVLTQSPATLSLSPGERATLSCRASQSISNNLHWYQQKPGQAPRLLIYYASRSISGIPARFSGSGSGTE |
| Protein | FTLTISSLQSEDFAVYYCQQSNSWPLTFGQGTKVEIK (SEQ ID NO: 38) |
| VL FR1 | EIVLTQSPATLSLSPGERATLSC (SEQ ID NO: 47) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GIPARFSGSGSGTEFTLTISSLQSEDFAVYYC (SEQ ID NO: 57) |
| VL CDR3 | QQSNSWPLT (SEQ ID NO: 44) |
| VL FR4 | FGQGTKVEIK (SEQ ID NO: 61) |
| VL DNA | GAAATTGTGTTGACACAGTCTCCAGCCACCCTGTCTTTGTCTCCAGGGGAAAGAGCCACCCTCTCCTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGATCCCAGCCAGGTTCAGTGGCAGTGGGTCTGGGACAGAG |
| | TTCACTCTCACCATCAGCAGCCTGCAGTCTGAAGATTTTGCAGTTTATTACTGTCAACAGAGTAACAGCT |
| | GGCCGCTCACGTTCGGCCAAGGGACCAAGGTGGAAATCAAA (SEQ ID NO: 67) |

X29

| | |
|---|---|
| VH | EVQLVQSGAEVKKPGESLRISCKGSGYTFTTYGMSWIRQSPSRGLEWLGWINTYSGVPTYADDFKGRFVF |
| Protein | SLDTSVSTAYLQICSLKAEDTAVYYCAREEYSSGYAAWFPYWGQGTLVTVSS (SEQ ID NO: 3) |
| VH FR1 | EVQLVQSGAEVKKPGESLRISCKGSGYTFT (SEQ ID NO: 14) |
| VH CDR1 | SYAIS (SEQ ID NO: 10) |
| VH FR2 | WIRQSPSRGLEWLG (SEQ ID NO: 16) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | GAAGTGCAGCTGGTGCAGTCTGGAGCAGAGGTGAAAAAGCCCGGGGAGTCTCTGAGGATCTCCTGTAAGG |
| | GTTCTGGGTATACCTTCACAACGTATGGAATGAGCTGGATCAGGCAGTCCCCATCGAGAGGCCTTGAGTG |
| | GCTGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 26) |
| VL | AIQLTQSPSSLSASVGDRVTITCRASQSISNNLHWYQQKPGQAPRLLIYYASRSISGVPSRFSGSGSGTE |
| Protein | FTLTISSLQPDDFATYYCQQSNSWPLTFGQGTKVEIK (SEQ ID NO: 36) |
| VL FR1 | AIQLTQSPSSLSASVGDRVTITC (SEQ ID NO: 46) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GVPSRFSGSGSGTEFTLTISSLQPDDFATYYC (SEQ ID NO: 55) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGGTCCCATCAAGGTTCAGCGGCAGTGGATCTGGGACAGAA |
| | TTCACTCTCACCATCAGCAGCCTGCAGCCTGATGATTTTGCAACTTATTACTGTCAACAGAGTAACAGCT |
| | GGCCGCTCACGTTCGGCCAAGGGACCAAGGTGGAAATCAAA (SEQ ID NO: 65) |

X9

| | |
|---|---|
| VH | EVQLVQSGAEVKKPGESLRISCKGSGYTFTTYGMSWVRQARGQRLEWIGWINTYSGVPTYADDFKGRFVF |
| Protein | SLDTSVSTAYLQICSLKAEDTAVYYCAREEYSSGYAAWFPYWGQGTLVTVSS (SEQ ID NO: 4) |
| VH FR1 | EVQLVQSGAEVKKPGESLRISCKGSGYTFT (SEQ ID NO: 14) |
| VH CDR1 | SYAIS (SEQ ID NO: 10) |
| VH FR2 | WVRQARGQRLEWIG (SEQ ID NO: 17) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | GAAGTGCAGCTGGTGCAGTCTGGAGCAGAGGTGAAAAAGCCCGGGGAGTCTCTGAGGATCTCCTGTAAGG |
| | GTTCTGGGTATACCTTCACAACGTATGGAATGAGCTGGGTGCGACAGGCTCGTGGACAACGCCTTGAGTG |
| | GATAGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 27) |
| VL | EIVLTQSPATLSLSPGERATLSCRASQSISNNLHWYLQKPGQSPQLLIYYASRSISGVPDRFSGSGSGTD |
| Protein | FTLKISRVEAEDVGVYYCQQSNSWPLTFGQGTKVEIK (SEQ ID NO: 39) |
| VL FR1 | EIVLTQSPATLSLSPGERATLSC (SEQ ID NO: 47) |
| VL CDR1 | RASQSISNNLH (SEQ ID NO: 42) |
| VL FR2 | WYLQKPGQSPQLLIY (SEQ ID NO: 51) |
| VL CDR2 | YASRSIS (SEQ ID NO: 43) |
| VL FR3 | GVPDRFSGSGSGTDFTLKISRVEAEDVGVYYC (SEQ ID NO: 58) |
| VL CDR3 | QQSNSWPLT (SEQ ID NO: 44) |
| VL FR4 | FGQGTKVEIK (SEQ ID NO: 61) |
| VL DNA | GAAATTGTGTTGACACAGTCTCCAGCCACCCTGTCTTTGTCTCCAGGGGAAAGAGCCACCCTCTCCTGCA |
| | GGGCCAGCCAAAGTATTAGCAACAACCTACACTGGTACCTGCAGAAGCCAGGGCAGTCTCCACAGCTCCT |
| | GATCTATTATGCTTCCCGGTCCATCTCTGGGGTCCCAGACAGGTTCAGTGGCAGTGGGTCAGGCACTGAT |
| | TTCACACTGAAAATCAGCAGGGTGGAGGCTGAGGATGTTGGAGTTTATTACTGTCAACAGAGTAACAGCT |
| | GGCCGCTCACGTTCGGCCAAGGGACCAAGGTGGAAATCAAA (SEQ ID NO: 68) |

X10

| | |
|---|---|
| VH | EVQLVQSGAEVKKPGESLRISCKGSGYTFTTYGMSWVRQATGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 5) |
| VH FR1 | EVQLVQSGAEVKKPGESLRISCKGSGYTFT (SEQ ID NO: 14) |
| VH CDR1 | SYAIS (SEQ ID NO: 10) |
| VH FR2 | WVRQATGQGLEWMG (SEQ ID NO: 18) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | GAAGTGCAGCTGGTGCAGTCTGGAGCAGAGGTGAAAAAGCCCGGGGAGTCTCTGAGGATCTCCTGTAAGG |
| | GTTCTGGGTATACCTTCACAACGTATGGAATGAGCTGGGTGCGACAGGCCACTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 28) |
| VL | EIVLTQSPATLSLSPGERATLSCRASQSISNNLHWYLQKPGQSPQLLIYYASRSISGVPDRFSGSGSGTD |
| Protein | FTLKISRVEAEDVGVYYCQQSNSWPLTFGQGTKVEIK (SEQ ID NO: 39) |
| VL FR1 | EIVLTQSPATLSLSPGERATLSC (SEQ ID NO: 47) |
| VL CDR1 | RASQSISNNLH (SEQ ID NO: 42) |
| VL FR2 | WYLQKPGQSPQLLIY (SEQ ID NO: 51) |
| VL CDR2 | YASRSIS (SEQ ID NO: 43) |
| VL FR3 | GVPDRFSGSGSGTDFTLKISRVEAEDVGVYYC (SEQ ID NO: 58) |
| VL CDR3 | QQSNSWPLT (SEQ ID NO: 44) |
| VL FR4 | FGQGTKVEIK (SEQ ID NO: 61) |
| VL DNA | GAAATTGTGTTGACACAGTCTCCAGCCACCCTGTCTTTGTCTCCAGGGGAAAGAGCCACCCTCTCCTGCA |
| | GGGCCAGCCAAAGTATTAGCAACAACCTACACTGGTACCTGCAGAAGCCAGGGCAGTCTCCACAGCTCCT |
| | GATCTATTATGCTTCCCGGTCCATCTCTGGGGTCCCAGACAGGTTCAGTGGCAGTGGGTCAGGCACTGAT |
| | TTCACACTGAAAATCAGCAGGGTGGAGGCTGAGGATGTTGGAGTTTATTACTGTCAACAGAGTAACAGCT |
| | GGCCGCTCACGTTCGGCCAAGGGACCAAGGTGGAAATCAAA (SEQ ID NO: 68) |

X11

| | |
|---|---|
| VH | EVQLVQSGAEVKKPGESLRISCKGSGYTFTTYGMSWVRQATGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 5) |
| VH FR1 | EVQLVQSGAEVKKPGESLRISCKGSGYTFT (SEQ ID NO: 14) |
| VH CDR1 | SYAIS (SEQ ID NO: 10) |
| VH FR2 | WVRQATGQGLEWMG (SEQ ID NO: 18) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | GAAGTGCAGCTGGTGCAGTCTGGAGCAGAGGTGAAAAAGCCCGGGGAGTCTCTGAGGATCTCCTGTAAGG |
| | GTTCTGGGTATACCTTCACAACGTATGGAATGAGCTGGGTGCGACAGGCCACTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 28) |
| VL | AIQLTQSPSSLSASVGDRVTITCRASQSISNNLHWYQQKPGKAPKLLIYYASRSISGVPSRFSGSGSGTE |
| Protein | FTLTISSLQPDDFATYYCQQSNSWPLTFGQGTKVEIK (SEQ ID NO: 35) |
| VL FR1 | AIQLTQSPSSLSASVGDRVTITC (SEQ ID NO: 45) |
| VL CDR1 | RASQSISNNLH (SEQ ID NO: 42) |
| VL FR2 | WYQQKPGKAPKLLIY (SEQ ID NO: 49) |
| VL CDR2 | YASRSIS (SEQ ID NO: 43) |
| VL FR3 | GVPSRFSGSGSGTEFTLTISSLQPDDFATYYC (SEQ ID NO: 55) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTATCAGCAGAAACCAGGGAAAGCTCCTAAGCTCCT |
| | GATCTATTATGCTTCCCGGTCCATCTCTGGGGTCCCATCAAGGTTCAGCGGCAGTGGATCTGGGACAGAA |
| | TTCACTCTCACCATCAGCAGCCTGCAGCCTGATGATTTTGCAACTTATTACTGTCAACAGAGTAACAGCT |
| | GGCCGCTCACGTTCGGCCAAGGGACCAAGGTGGAAATCAAA (SEQ ID NO: 64) |

X24

| | |
|---|---|
| VH | EVQLVQSGAEVKKPGESLRISCKGSGYTFTTYGMSWVRQATGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 5) |
| VH FR1 | EVQLVQSGAEVKKPGESLRISCKGSGYTFT (SEQ ID NO: 14) |
| VH CDR1 | SYAIS (SEQ ID NO: 10) |
| VH FR2 | WVRQATGQGLEWMG (SEQ ID NO: 18) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | GAAGTGCAGCTGGTGCAGTCTGGAGCAGAGGTGAAAAAGCCCGGGGAGTCTCTGAGGATCTCCTGTAAGG |
| | GTTCTGGGTATACCTTCACAACGTATGGAATGAGCTGGGTGCGACAGGCCACTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 28) |
| VL | EIVLTQSPATLSLSPGERATLSCRASQSISNNLHWYQQKPGQAPRLLIYYASRSISGIPARFSGSGSGTE |
| Protein | FTLTISSLQSEDFAVYYCQQSNSWPLTFGQGTKVEIK (SEQ ID NO: 38) |
| VL FR1 | EIVLTQSPATLSLSPGERATLSC (SEQ ID NO: 47) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GIPARFSGSGSGTEFTLTISSLQSEDFAVYYC (SEQ ID NO: 57) |
| VL CDR3 | QQSNSWPLT (SEQ ID NO: 44) |
| VL FR4 | FGQGTKVEIK (SEQ ID NO: 61) |
| VL DNA | GAAATTGTGTTGACACAGTCTCCAGCCACCCTGTCTTTGTCTCCAGGGGAAAGAGCCACCCTCTCCTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGATCCCAGCCAGGTTCAGTGGCAGTGGGTCTGGGACAGAG |
| | TTCACTCTCACCATCAGCAGCCTGCAGTCTGAAGATTTTGCAGTTTATTACTGTCAACAGAGTAACAGCT |
| | GGCCGCTCACGTTCGGCCAAGGGACCAAGGTGGAAATCAAA (SEQ ID NO: 67) |

X25

| | |
|---|---|
| VH | EVQLVQSGAEVKKPGESLRISCKGSGYTFTTYGMSWVRQATGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 5) |
| VH FR1 | EVQLVQSGAEVKKPGESLRISCKGSGYTFT (SEQ ID NO: 14) |
| VH CDR1 | SYAIS (SEQ ID NO: 10) |
| VH FR2 | WVRQATGQGLEWMG (SEQ ID NO: 18) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | GAAGTGCAGCTGGTGCAGTCTGGAGCAGAGGTGAAAAAGCCCGGGGAGTCTCTGAGGATCTCCTGTAAGG |
| | GTTCTGGGTATACCTTCACAACGTATGGAATGAGCTGGGTGCGACAGGCCACTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 28) |
| VL | AIQLTQSPSSLSASVGDRVTITCRASQSISNNLHWYQQKPGQAPRLLIYYASRSISGVPSRFSGSGSGTE |
| Protein | FTLTISSLQPDDFATYYCQQSNSWPLTFGQGTKVEIK (SEQ ID NO: 36) |
| VL FR1 | AIQLTQSPSSLSASVGDRVTITC (SEQ ID NO: 46) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GVPSRFSGSGSGTEFTLTISSLQPDDFATYYC (SEQ ID NO: 55) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGGTCCCATCAAGGTTCAGCGGCAGTGGATCTGGGACAGAA |
| | TTCACTCTCACCATCAGCAGCCTGCAGCCTGATGATTTTGCAACTTATTACTGTCAACAGAGTAACAGCT |
| | GGCCGCTCACGTTCGGCCAAGGGACCAAGGTGGAAATCAAA (SEQ ID NO: 65) |

X15

| | |
|---|---|
| VH | QVQLVQSGAEVKKPGASVKVSCKASGYTFTTYGMSWVRQARGQRLEWIGWINTYSGVPTYADDFKGRFVF |
| Protein | SLDTSVSTAYLQICSLKAEDTAVYYCAREEYSSGYAAWFPYWGQGTLVTVSS (SEQ ID NO: 6) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WVRQARGQRLEWIG (SEQ ID NO: 17) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGGTGCGACAGGCCCGTGGACAACGCCTTGAGTG |
| | GATAGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 29) |
| VL | AIQLTQSPSSLSASVGDRVTITCRASQSISNNLHWYQQKPGKAPKLLIYYASRSISGVPSRFSGSGSGTE |
| Protein | FTLTISSLQPDDFATYYCQQSNSWPLTFGQGTKVEIK (SEQ ID NO: 35) |
| VL FR1 | AIQLTQSPSSLSASVGDRVTITC (SEQ ID NO: 45) |
| VL CDR1 | RASQSISNNLH (SEQ ID NO: 42) |
| VL FR2 | WYQQKPGKAPKLLIY (SEQ ID NO: 49) |
| VL CDR2 | YASRSIS (SEQ ID NO: 43) |
| VL FR3 | GVPSRFSGSGSGTEFTLTISSLQPDDFATYYC (SEQ ID NO: 55) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTATCAGCAGAAACCAGGGAAAGCTCCTAAGCTCCT |
| | GATCTATTATGCTTCCCGGTCCATCTCTGGGGTCCCATCAAGGTTCAGCGGCAGTGGATCTGGGACAGAA |
| | TTCACTCTCACCATCAGCAGCCTGCAGCCTGATGATTTTGCAACTTATTACTGTCAACAGAGTAACAGCT |
| | GGCCGCTCACGTTCGGCCAAGGGACCAAGGTGGAAATCAAA (SEQ ID NO: 64) |

X26

| | |
|---|---|
| VH | QVQLVQSGAEVKKPGASVKVSCKASGYTFTTYGMSWVRQARGQRLEWIGWINTYSGVPTYADDFKGRFVF |
| Protein | SLDTSVSTAYLQICSLKAEDTAVYYCAREEYSSGYAAWFPYWGQGTLVTVSS (SEQ ID NO: 6) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WVRQARGQRLEWIG (SEQ ID NO: 17) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGGTGCGACAGGCCCGTGGACAACGCCTTGAGTG |
| | GATAGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 29) |
| VL | EIVLTQSPATLSLSPGERATLSCRASQSISNNLHWYQQKPGQAPRLLIYYASRSISGIPARFSGSGSGTE |
| Protein | FTLTISSLQSEDFAVYYCQQSNSWPLTFGQGTKVEIK (SEQ ID NO: 38) |
| VL FR1 | EIVLTQSPATLSLSPGERATLSC (SEQ ID NO: 47) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GIPARFSGSGSGTEFTLTISSLQSEDFAVYYC (SEQ ID NO: 57) |
| VL CDR3 | QQSNSWPLT (SEQ ID NO: 44) |
| VL FR4 | FGQGTKVEIK (SEQ ID NO: 61) |
| VL DNA | GAAATTGTGTTGACACAGTCTCCAGCCACCCTGTCTTTGTCTCCAGGGGAAAGAGCCACCCTCTCCTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGATCCCAGCCAGGTTCAGTGGCAGTGGGTCTGGGACAGAG |
| | TTCACTCTCACCATCAGCAGCCTGCAGTCTGAAGATTTTGCAGTTTATTACTGTCAACAGAGTAACAGCT |
| | GGCCGCTCACGTTCGGCCAAGGGACCAAGGTGGAAATCAAA (SEQ ID NO: 67) |

X27

| | |
|---|---|
| VH | QVQLVQSGAEVKKPGASVKVSCKASGYTFTTYGMSWVRQARGQRLEWIGWINTYSGVPTYADDFKGRFVF |
| Protein | SLDTSVSTAYLQICSLKAEDTAVYYCAREEYSSGYAAWFPYWGQGTLVTVSS (SEQ ID NO: 6) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WVRQARGQRLEWIG (SEQ ID NO: 17) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGGTGCGACAGGCCCGTGGACAACGCCTTGAGTG |
| | GATAGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 29) |
| VL | AIQLTQSPSSLSASVGDRVTITCRASQSISNNLHWYQQKPGQAPRLLIYYASRSISGVPSRFSGSGSGTE |
| Protein | FTLTISSLQPDDFATYYCQQSNSWPLTFGQGTKVEIK (SEQ ID NO: 36) |
| VL FR1 | AIQLTQSPSSLSASVGDRVTITC (SEQ ID NO: 46) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GVPSRFSGSGSGTEFTLTISSLQPDDFATYYC (SEQ ID NO: 55) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGGTCCCATCAAGGTTCAGCGGCAGTGGATCTGGGACAGAA |
| | TTCACTCTCACCATCAGCAGCCTGCAGCCTGATGATTTTGCAACTTATTACTGTCAACAGAGTAACAGCT |
| | GGCCGCTCACGTTCGGCCAAGGGACCAAGGTGGAAATCAAA (SEQ ID NO: 65) |

X16

| | |
|---|---|
| VH | QVQLVQSGAEVKKPGASVKVSCKASGYTFTTYGMSWVRQATGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 7) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WVRQATGQGLEWMG (SEQ ID NO: 18) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGGTGCGACAGGCCACTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 30) |
| VL | AIQLTQSPSSLSASVGDRVTITCRASQSISNNLHWYQQKPGKAPKLLIYYASRSISGVPSRFSGSGSGTE |
| Protein | FTLTISSLQPDDFATYYCQQSNSWPLTFGQGTKVEIK (SEQ ID NO: 35) |
| VL FR1 | AIQLTQSPSSLSASVGDRVTITC (SEQ ID NO: 45) |
| VL CDR1 | RASQSISNNLH (SEQ ID NO: 42) |
| VL FR2 | WYQQKPGKAPKLLIY (SEQ ID NO: 49) |
| VL CDR2 | YASRSIS (SEQ ID NO: 43) |
| VL FR3 | GVPSRFSGSGSGTEFTLTISSLQPDDFATYYC (SEQ ID NO: 55) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTATCAGCAGAAACCAGGGAAAGCTCCTAAGCTCCT |
| | GATCTATTATGCTTCCCGGTCCATCTCTGGGGTCCCATCAAGGTTCAGCGGCAGTGGATCTGGGACAGAA |
| | TTCACTCTCACCATCAGCAGCCTGCAGCCTGATGATTTTGCAACTTATTACTGTCAACAGAGTAACAGCT |
| | GGCCGCTCACGTTCGGCCAAGGGACCAAGGTGGAAATCAAA (SEQ ID NO: 64) |

X17

| | |
|---|---|
| VH | QVQLVQSGAEVKKPGASVKVSCKASGYTFTTYGMSWVRQATGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 7) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WVRQATGQGLEWMG (SEQ ID NO: 18) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGGTGCGACAGGCCACTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCTGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 30) |
| VL | AIQLTQSPSSLSASVGDRVTITCRASQSISNNLHWYQQKPGQAPRLLIYYASRSISGVPSRFSGSGSGTD |
| Protein | FTFTISSLQPEDIATYYCQQSNSWPLTFGQGTKVEIK (SEQ ID NO: 34) |
| VL FR1 | AIQLTQSPSSLSASVGDRVTITC (SEQ ID NO: 46) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GVPSRFSGSGSGTDFTFTISSLQPEDIATYYC (SEQ ID NO: 54) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGGTCCCATCAAGGTTCAGTGGAAGTGGATCTGGGACAGAT |
| | TTTACTTTCACCATCAGCAGCCTGCAGCCTGAAGATATTGCAACATATTACTGTCAACAGAGTAACAGCT |
| | GGCCGCTCACGTTCGGCCAAGGGACCAAGGTGGAAATCAAA (SEQ ID NO: 63) |

X18

| | |
|---|---|
| VH | QVQLVQSGAEVKKPGASVKVSCKASGYTFTTYGMSWVRQAPGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 8) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WVRQAPGQGLEWMG (SEQ ID NO: 19) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGGTGCGACAGGCCCCTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCAGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 31) |
| VL | AIQLTQSPSSLSASVGDRVTITCRASQSISNNLHWYQQKPGKAPKLLIYYASRSISGVPSRFSGSGSGTE |
| Protein | FTLTISSLQPDDFATYYCQQSNSWPLTFGQGTKVEIK (SEQ ID NO: 35) |
| VL FR1 | AIQLTQSPSSLSASVGDRVTITC (SEQ ID NO: 45) |
| VL CDR1 | RASQSISNNLH (SEQ ID NO: 42) |
| VL FR2 | WYQQKPGKAPKLLIY (SEQ ID NO: 49) |
| VL CDR2 | YASRSIS (SEQ ID NO: 43) |
| VL FR3 | GVPSRFSGSGSGTEFTLTISSLQPDDFATYYC (SEQ ID NO: 55) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTATCAGCAGAAACCAGGGAAAGCTCCTAAGCTCCT |
| | GATCTATTATGCTTCCCGGTCCATCTCTGGGGTCCCATCAAGGTTCAGCGGCAGTGGATCTGGGACAGAA |
| | TTCACTCTCACCATCAGCAGCCTGCAGCCTGATGATTTTGCAACTTATTACTGTCAACAGAGTAACAGCT |
| | GGCCGCTCACGTTCGGCCAAGGGACCAAGGTGGAAATCAAA (SEQ ID NO: 64) |

X19

| | |
|---|---|
| VH | QVQLVQSGAEVKKPGASVKVSCKASGYTFTTYGMSWVRQAPGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 8) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WVRQAPGQGLEWMG (SEQ ID NO: 19) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGGTGCGACAGGCCCCTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCAGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 31) |
| VL | EIVLTQSPDFQSVTPKEKVTITCRASQSISNNLHWYQQKPGKAPKLLIYYASRSISGIPPRFSGSGYGTD |
| Protein | FTLTINNIESEDAAYYFCQQSNSWPLTFGQGTKVEIK (SEQ ID NO: 33) |
| VL FR1 | EIVLTQSPDFQSVTPKEKVTITC (SEQ ID NO: 45) |
| VL CDR1 | RASQSISNNLH (SEQ ID NO: 42) |
| VL FR2 | WYQQKPGKAPKLLIY (SEQ ID NO: 49) |
| VL CDR2 | YASRSIS (SEQ ID NO: 43) |
| VL FR3 | GIPPRFSGSGYGTDFTLTINNIESEDAAYYFC (SEQ ID NO: 53) |
| VL CDR3 | QQSNSWPLT (SEQ ID NO: 44) |
| VL FR4 | FGQGTKVEIK (SEQ ID NO: 61) |
| VL DNA | GAAATTGTGCTGACTCAGTCTCCAGACTTTCAGTCTGTGACTCCAAAGGAGAAAGTCACCATCACCTGCA |
| | GGGCCAGCCAAAGTATTAGCAACAACCTACACTGGTATCAGCAGAAACCAGGGAAAGCTCCTAAGCTCCT |
| | GATCTATTATGCTTCCCGGTCCATCTCTGGGATCCCACCTCGATTCAGTGGCAGCGGGTATGGAACAGAT |
| | TTTACCCTCACAATTAATAACATAGAATCTGAGGATGCTGCATATTACTTCTGTCAACAGAGTAACAGCT |
| | GGCCGCTCACGTTCGGCCAAGGGACCAAGGTGGAAATCAAA (SEQ ID NO: 62) |

X21

| | |
|---|---|
| VH | QVQLVQSGAEVKKPGASVKVSCKASGYTFTTYGMSWVRQAPGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 8) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WVRQAPGQGLEWMG (SEQ ID NO: 19) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGGTGCGACAGGCCCCTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCAGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 31) |
| VL | EIVLTQSPATLSLSPGERATLSCRASQSISNNLHWYQQKPGQAPRLLIYYASRSISGVPSRFSGSGSGTD |
| Protein | FTFTISSLEAEDAATYYCQQSNSWPLTFGQGTKVEIK (SEQ ID NO: 37) |
| VL FR1 | EIVLTQSPATLSLSPGERATLSC (SEQ ID NO: 47) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GVPSRFSGSGSGTDFTFTISSLEAEDAATYYC (SEQ ID NO: 56) |
| VL CDR3 | QQSNSWPLT (SEQ ID NO: 44) |
| VL FR4 | FGQGTKVEIK (SEQ ID NO: 61) |
| VL DNA | GAAATTGTGTTGACACAGTCTCCAGCCACCCTGTCTTTGTCTCCAGGGGAAAGAGCCACCCTCTCCTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGGTCCCCTCGAGGTTCAGTGGCAGTGGATCTGGGACAGAT |
| | TTCACCTTTACCATCAGTAGCCTGGAAGCTGAAGATGCTGCAACATATTACTGTCAACAGAGTAACAGCT |
| | GGCCGCTCACGTTCGGCCAAGGGACCAAGGTGGAAATCAAA (SEQ ID NO: 66) |

X22

| | |
|---|---|
| VH | QVQLVQSGAEVKKPGASVKVSCKASGYTFTTYGMSWVRQAPGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 8) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WVRQAPGQGLEWMG (SEQ ID NO: 19) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGGTGCGACAGGCCCCTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCAGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 31) |
| VL | AIQLTQSPSSLSASVGDRVTITCRASQSISNNLHWYQQKPGQAPRLLIYYASRSISGVPSRFSGSGSGTD |
| Protein | FTFTISSLQPEDIATYYCQQSNSWPLTFGQGTKVEIK (SEQ ID NO: 34) |
| VL FR1 | AIQLTQSPSSLSASVGDRVTITC (SEQ ID NO: 46) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GVPSRFSGSGSGTDFTFTISSLQPEDIATYYC (SEQ ID NO: 54) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGGTCCCATCAAGGTTCAGTGGAAGTGGATCTGGGACAGAT |
| | TTTACTTTCACCATCAGCAGCCTGCAGCCTGAAGATATTGCAACATATTACTGTCAACAGAGTAACAGCT |
| | GGCCGCTCACGTTCGGCCAAGGGACCAAGGTGGAAATCAAA (SEQ ID NO: 63) |

X23

| | |
|---|---|
| VH | QVQLVQSGAEVKKPGASVKVSCKASGYTFTTYGMSWVRQAPGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 8) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WVRQAPGQGLEWMG (SEQ ID NO: 19) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGGTGCGACAGGCCCCTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCAGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 31) |
| VL | EIVLTQSPDFQSVTPKEKVTITCRASQSISNNLHWYLQKPGQSPQLLIYYASRSISGVPDRFSGSGSGTD |
| Protein | FTLKISRVEAEDVGVYYCQQSNSWPLTFGQGTKVEIK (SEQ ID NO: 40) |
| VL FR1 | EIVLTQSPDFQSVTPKEKVTITC (SEQ ID NO: 45) |
| VL CDR1 | RASQSISNNLH (SEQ ID NO: 42) |
| VL FR2 | WYLQKPGQSPQLLIY (SEQ ID NO: 57) |
| VL CDR2 | YASRSIS (SEQ ID NO: 43) |
| VL FR3 | GVPDRFSGSGSGTDFTLKISRVEAEDVGVYYC (SEQ ID NO: 59) |
| VL CDR3 | QQSNSWPLT (SEQ ID NO: 44) |
| VL FR4 | FGQGTKVEIK (SEQ ID NO: 61) |
| VL DNA | GAAATTGTGCTGACTCAGTCTCCAGACTTTCAGTCTGTGACTCCAAAGGAGAAAGTCACCATCACCTGCA |
| | GGGCCAGCCAAAGTATTAGCAACAACCTACACTGGTACCTGCAGAAGCCAGGGCAGTCTCCACAGCTCCT |
| | GATCTATTATGCTTCCCGGTCCATCTCTGGGGTCCCAGACAGGTTCAGTGGCAGTGGGTCAGGCACTGAT |
| | TTCACACTGAAAATCAGCAGGGTGGAGGCTGAGGATGTTGGAGTTTATTACTGTCAACAGAGTAACAGCT |
| | GGCCGCTCACGTTCGGCCAAGGGACCAAGGTGGAAATCAAA (SEQ ID NO: 69) |

X30

| | |
|---|---|
| VH | QVQLVQSGAEVKKPGASVKVSCKASGYTFTTYGMSWVRQAPGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 8) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WVRQAPGQGLEWMG (SEQ ID NO: 19) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGGTGCGACAGGCCCCTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCAGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 31) |
| VL | EIVLTQSPATLSLSPGERATLSCRASQSISNNLHWYQQKPGQAPRLLIYYASRSISGIPARFSGSGSGTE |
| Protein | FTLTISSLQSEDFAVYYCQQSNSWPLTFGQGTKVEIK (SEQ ID NO: 38) |
| VL FR1 | EIVLTQSPATLSLSPGERATLSC (SEQ ID NO: 47) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GIPARFSGSGSGTEFTLTISSLQSEDFAVYYC (SEQ ID NO: 57) |
| VL CDR3 | QQSNSWPLT (SEQ ID NO: 44) |
| VL FR4 | FGQGTKVEIK (SEQ ID NO: 61) |
| VL DNA | GAAATTGTGTTGACACAGTCTCCAGCCACCCTGTCTTTGTCTCCAGGGGAAAGAGCCACCCTCTCCTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGATCCCAGCCAGGTTCAGTGGCAGTGGGTCTGGGACAGAG |
| | TTCACTCTCACCATCAGCAGCCTGCAGTCTGAAGATTTTGCAGTTTATTACTGTCAACAGAGTAACAGCT |
| | GGCCGCTCACGTTCGGCCAAGGGACCAAGGTGGAAATCAAA (SEQ ID NO: 67) |

X31

| | |
|---|---|
| VH | QVQLVQSGAEVKKPGASVKVSCKASGYTFTTYGMSWVRQAPGQGLEWMGWINTYSGVPTYADDFKGRFVF |
| Protein | SLDTSVSTAYLQICSLKAEDTAVYYCAREEYSSGYAAWFPYWGQGTLVTVSS (SEQ ID NO: 8) |
| VH FR1 | QVQLVQSGAEVKKPGASVKVSCKASGYTFT (SEQ ID NO: 13) |
| VH CDR1 | SYAIS (SEQ ID NO: 10) |
| VH FR2 | WVRQAPGQGLEWMG (SEQ ID NO: 19) |
| VH CDR2 | GIIPIFGTASYAQKFQG (SEQ ID NO: 11) |
| VH FR3 | RFVFSLDTSVSTAYLQICSLKAEDTAVYYCAR (SEQ ID NO: 21) |
| VH CDR3 | TPYYDSSGYLDV (SEQ ID NO: 12) |
| VH FR4 | WGQGTLVTVSS (SEQ ID NO: 23) |
| VH DNA | CAGGTTCAGCTGGTGCAGTCTGGAGCTGAGGTGAAGAAGCCTGGGGCCTCAGTGAAGGTCTCCTGCAAGG |
| | CTTCTGGGTATACCTTCACAACGTATGGAATGAGCTGGGTGCGACAGGCCCCTGGACAAGGGCTTGAGTG |
| | GATGGGTTGGATAAACACCTACTCTGGAGTGCCAACATATGCTGATGACTTCAAGGGAAGATTTGTCTTC |
| | TCCTTGGACACCTCTGTCAGCACGGCATATCTGCAGATCTGCAGCCTAAAGGCAGAGGACACTGCCGTGT |
| | ATTACTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 31) |
| VL | AIQLTQSPSSLSASVGDRVTITCRASQSISNNLHWYQQKPGQAPRLLIYYASRSISGVPSRFSGSGSGTE |
| Protein | FTLTISSLQPDDFATYYCQQSNSWPLTFGQGTKVEIK (SEQ ID NO: 36) |
| VL FR1 | AIQLTQSPSSLSASVGDRVTITC (SEQ ID NO: 46) |
| VL CDR1 | RASQSISNNLH (SEQ ID NO: 42) |
| VL FR2 | WYQQKPGQAPRLLIY (SEQ ID NO: 50) |
| VL CDR2 | YASRSIS (SEQ ID NO: 43) |
| VL FR3 | GVPSRFSGSGSGTEFTLTISSLQPDDFATYYC (SEQ ID NO: 55) |
| VL CDR3 | QQSNSWPLT (SEQ ID NO: 44) |
| VL FR4 | FGQGTKVEIK (SEQ ID NO: 61) |
| VL DNA | GCCATCCAGTTGACCCAGTCTCCATCCTCCCTGTCTGCATCTGTAGGAGACAGAGTCACCATCACTTGCA |
| | GGGCCAGCCAAAGTATTAGCAACAACCTACACTGGTACCAGCAGAAACCTGGCCAGGCTCCCAGGCTCCT |
| | CATCTATTATGCTTCCCGGTCCATCTCTGGGGTCCCATCAAGGTTCAGCGGCAGTGGATCTGGGACAGAA |
| | TTCACTCTCACCATCAGCAGCCTGCAGCCTGATGATTTTGCAACTTATTACTGTCAACAGAGTAACAGCT |
| | GGCCGCTCACGTTCGGCCAAGGGACCAAGGTGGAAATCAAA (SEQ ID NO: 65) |

TABLE 2
Exemplary 1C11 mAb Optimization

| | |
|---|---|
| VH | QIQLVQSGPELKKPGETVKISCKASGYTFTTYGMSWVNQAPGKGLKWMGWINTYSGVPTYADDFKGRFVF |
| Protein | SLETSASTAYLQINNLKNEDTATYFCAREEYSSGYAAWFPYWGQGTLVTVSS (SEQ ID NO: 9) |
| | MDMRVPAQLLGLLLLWLPGAKCQIQLVQSGPELKKPGETVKISCKASGYTFTTYGMSWVNQAPGKGLKWM |
| | GWINTYSGVPTYADDFKGRFVFSLETSASTAYLQINNLKNEDTATYFCAREEYSSGYAAWFPYWGQGTLV |
| | TVSSASTKGPSVFPLAPSSKSTSGGTAALGCLVKDYFPEPVTVSWNSGALTSGVHTFPAVLQSSGLYSLS |
| | SVVTVPSSSLGTQTYICNVNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSLSPGK (SEQ ID NO: 76) |
| VH FR1 | QIQLVQSGPELKKPGETVKISCKASGYTFT (SEQ ID NO: 15) |
| VH CDR1 | SYAIS (SEQ ID NO: 10) |
| VH FR2 | WVNQAPGKGLKWMG (SEQ ID NO: 20) |
| VH CDR2 | GIIPIFGTASYAQKFQG (SEQ ID NO: 11) |
| VH FR3 | RFVFSLETSASTAYLQINNLKNEDTATYFCAR (SEQ ID NO: 22) |
| VH CDR3 | TPYYDSSGYLDV (SEQ ID NO: 12) |
| VH FR4 | WGQGTLVTVSS (SEQ ID NO: 23) |
| VH DNA | CAGATCCAGTTGGTACAGTCTGGACCTGAGCTGAAGAAGCCTGGAGAGACAGTCAAGATCTCCTGCAAGG |
| | CTTCTGGGTATACCTTCACAACGTATGGAATGAGCTGGGTGAATCAGGCTCCAGGAAAGGGTTTAAAGTG |
| | GATGGGCTGGATAAACACCTACTCTGGAGTGCCAACATATGCTGATGACTTCAAGGGACGGTTTGTCTTC |
| | TCTTTGGAAACCTCTGCCAGCACTGCCTATTTGCAGATCAACAACCTCAAAAATGAGGACACGGCTACAT |
| | ATTTCTGTGCAAGAGAGGAGTACAGCTCAGGCTACGCGGCCTGGTTTCCTTACTGGGGCCAGGGAACGCT |
| | GGTCACCGTCAGCTCA (SEQ ID NO: 32) |
| | ATGGACATGAGGGTCCCCGCTCAGCTCCTGGGGCTCCTGCTGCTCTGGCTCCCAGGTGCCAAGTGTCAGA |
| | TCCAGTTGGTACAGTCTGGACCTGAGCTGAAGAAGCCTGGAGAGACAGTCAAGATCTCCTGCAAGGCTTC |
| | TGGGTATACCTTCACAACGTATGGAATGAGCTGGGTGAATCAGGCTCCAGGAAAGGGTTTAAAGTGGATG |
| | GGCTGGATAAACACCTACTCTGGAGTGCCAACATATGCTGATGACTTCAAGGGACGGTTTGTCTTCTCTT |
| | TGGAAACCTCTGCCAGCACTGCCTATTTGCAGATCAACAACCTCAAAAATGAGGACACGGCTACATATTT |
| | CTGTGCAAGAGAGGAGTACAGCTCAGGCTACGCGGCCTGGTTTCCTTACTGGGGCCAGGGAACGCTGGTC |
| | ACCGTCAGCTCAGCCTCCACCAAGGGCCCATCGGTCTTCCCCCTGGCACCCTCCTCCAAGAGCACCTCTG |
| | GGGGCACAGCGGCCCTGGGCTGCCTGGTCAAGGACTACTTCCCCGAACCGGTGACGGTGTCGTGGAACTC |
| | AGGCGCCCTGACCAGCGGCGTGCACACCTTCCCGGCTGTCCTACAGTCCTCAGGACTCTACTCCCTCAGC |
| | AGCGTGGTGACCGTGCCCTCCAGCAGCTTGGGCACCCAGACCTACATCTGCAACGTGAATCACAAGCCCA |
| | GCAACACCAAGGTGGACAAGAAAGTTGAGCCCAAATCTTGTGACAAAACTCACACATGCCCACCGTGCCC |
| | AGCACCTGAACTCCTGGGGGGACCGTCAGTCTTCCTCTTCCCCCCAAAACCCAAGGACACCCTCATGATC |
| | TCCCGGACCCCTGAGGTCACATGCGTGGTGGTGGACGTGAGCCACGAAGACCCTGAGGTCAAGTTCAACT |
| | GGTACGTGGACGGCGTGGAGGTGCATAATGCCAAGACAAAGCCGCGGGAGGAGCAGTACAACAGCACGTA |
| | CCGTGTGGTCAGCGTCCTCACCGTCCTGCACCAGGACTGGCTGAATGGCAAGGAGTACAAGTGCAAGGTC |
| | AGCAACAAAGCCCTCCCAGCCCCCATCGAGAAAACCATCTCCAAAGCCAAAGGGCAGCCCCGAGAACCAC |
| | AGGTGTACACCCTGCCCCCATCCCGGGATGAGCTGACCAAGAACCAGGTCAGCCTGACCTGCCTGGTCAA |
| | AGGCTTCTATCCCAGCGACATCGCCGTGGAGTGGGAGAGCAATGGGCAGCCGGAGAACAACTACAAGACC |
| | ACGCCTCCCGTGCTGGACTCCGACGGCTCCTTCTTCCTCTACAGCAAGCTCACCGIGGACAAGAGCAGGT |
| | GGCAGCAGGGGAACGTCTTCTCATGCTCCGTGATGCATGAGGCTCTGCACAACCACTACACGCAGAAGAG |
| | CCTCTCCCTGTCTCCGGGTAAATGA (SEQ ID NO: 73) |
| VL | DIVLTQSPATLSVTPGDSVSLSCRASQSISNNLHWYQQKSHESPRLLIEYASRSISGIPSRFSGGGSGTD |
| Protein | FTLSINSVESEDFGLYFCQQSNSWPLTFGQGTKVEIK (SEQ ID NO: 41) |
| | MDMRVPAQLLGLLLLWLPGAKCDIVLTQSPATLSVTPGDSVSLSCRASQSISNNLHWYQQKSHESPRLLI |
| | EYASRSISGIPSRFSGGGSGTDFTLSINSVESEDFGLYFCQQSNSWPLTFGQGTKVEIKRTVAAPSVFIF |
| | PPSDEQLKSGTASVVCLLNNFYPREAKVQWKVDNALQSGNSQESVTEQDSKDSTYSLSSTLTLSKADYEK |
| | HKVYACEVTHQGLSSPVTKSFNRGEC (SEQ ID NO: 75) |
| VL FR1 | IVLTQSPATLSVTPGDSVSLSC (SEQ ID NO: 48) |
| VL CDR1 | RASQSISNNLH (SEQ ID NO: 42) |
| VL FR2 | WYQQKSHESPRLLIE (SEQ ID NO: 52) |
| VL CDR2 | YASRSIS (SEQ ID NO: 43) |
| VL FR3 | GIPSRFSGGGSGTDFTLSINSVESEDFGLYFC (SEQ ID NO: 60) |
| VL CDR3 | QQSNSWPLT (SEQ ID NO: 44) |
| VL FR4 | FGQGTKVEIK (SEQ ID NO: 61) |
| VL DNA | GATATTGTGCTAACTCAGTCTCCAGCCACCCTGTCTGTGACTCCAGGAGATAGCGTCAGTCTTTCCTGCA |
| | GGGCCAGCCAAAGTATTAGCAACAACCTACACTGGTATCAACAAAAATCACATGAGTCTCCAAGGCTTCT |
| | CATCGAATATGCTTCCCGGTCCATCTCTGGGATCCCCTCTAGGTTCAGTGGCGGTGGATCAGGGACAGAT |
| | TTCACTCTCAGTATCAACAGTGTGGAGTCTGAAGATTTTGGATTGTATTTCTGTCAACAGAGTAACAGCT |
| | GGCCGCTCACGTTCGGCCAAGGGACCAAGGTGGAAATCAAA (SEQ ID NO: 70) |
| | ATGGACATGAGGGTCCCCGCTCAGCTCCTGGGGCTCCTGCTGCTCTGGCTCCCAGGTGCCAAGTGTGATA |
| | TTGTGCTAACTCAGTCTCCAGCCACCCTGTCTGTGACTCCAGGAGATAGCGTCAGTCTTTCCTGCAGGGC |
| | CAGCCAAAGTATTAGCAACAACCTACACTGGTATCAACAAAAATCACATGAGTCTCCAAGGCTTCTCATC |
| | GAATATGCTTCCCGGTCCATCTCTGGGATCCCCTCTAGGTTCAGTGGCGGTGGATCAGGGACAGATTTCA |
| | CTCTCAGTATCAACAGTGTGGAGTCTGAAGATTTTGGATTGTATTTCTGTCAACAGAGTAACAGCTGGCC |
| | GCTCACGTTCGGCCAAGGGACCAAGGTGGAAATCAAACGTACGGTGGCTGCACCATCTGTCTTCATCTTC |
| | CCGCCATCTGATGAGCAGTTGAAATCTGGAACTGCCTCTGTTGTGTGCCTGCTGAATAACTTCTATCCCA |
| | GAGAGGCCAAAGTACAGTGGAAGGTGGATAACGCCCTCCAATCGGGTAACTCCCAGGAGAGTGTCACAGA |
| | GCAGGACAGCAAGGACAGCACCTACAGCCTCAGCAGCACCCTGACGCTGAGCAAAGCAGACTACGAGAAA |
| | CACAAAGTCTACGCCTGCGAAGTCACCCATCAGGGCCTGAGCTCGCCCGTCACAAAGAGCTTCAACAGGG |
| | GAGAGTGTTGA (SEQ ID NO: 74) |

1. An isolated Gmd-binding antibody which comprises a VH comprising a VH-CDR1 of SEQ ID NO:10, a VH-CDR2 of SEQ ID NO:11, and a VH-CDR3 of SEQ ID NO: 12, and VL comprising a VH-CDR1 of SEQ ID NO:42, a VH-CDR2 of SEQ ID NO: 43, and a VH-CDR3 of SEQ ID NO:44, and comprises a framework sequence selected from the group consisting of
(a) a VH framework 1 sequence of SEQ ID NO:13 or 14;
(b) a VH framework 2 sequence of SEQ ID NO:16-18, or 19;
(c) a VH framework 3 sequence of SEQ ID NO:21;
(d) a VL framework 1 sequence of SEQ ID NO:45, 46, or 47;
(e) a VL framework 2 sequence of SEQ ID NO:49, 50, or 51; and
(f) a VL framework 3 sequence of SEQ ID NO:53-58, or 59;
or a VL framework sequences selected from the group consisting of:
(a) a framework 1 sequence of SEQ ID NO:45 and a framework 2 sequence of SEQ ID NO:49;
(b) a framework 1 sequence of SEQ ID NO:46 and a framework 2 sequence of SEQ ID NO:50;
(c) a framework 1 sequence of SEQ ID NO:47 and a framework 2 sequence of SEQ ID NO:50;
(d) a framework 1 sequence of SEQ ID NO:47 and a framework 2 sequence of SEQ ID NO:51;
(e) a framework 2 sequence of SEQ ID NO:49 and a framework 3 sequence of SEQ ID NO:53;
(f) a framework 2 sequence of SEQ ID NO:49 and a framework 3 sequence of SEQ ID NO:55;
(g) a framework 2 sequence of SEQ ID NO:50 and a framework 3 sequence of SEQ ID NO:54;
(h) a framework 2 sequence of SEQ ID NO:50 and a framework 3 sequence of SEQ ID NO:55;
(i) a framework 2 sequence of SEQ ID NO:50 and a framework 3 sequence of SEQ ID NO:56;
(j) a framework 2 sequence of SEQ ID NO:50 and a framework 3 sequence of SEQ ID NO:57; and
(k) a framework 2 sequence of SEQ ID NO:51 and a framework 3 sequence of SEQ ID NO:58.

2. The Gmd-binding antibody of claim 1 which comprises VH framework sequences selected from the group consisting of:
(a) a framework 1 sequence of SEQ ID NO:13, a framework 2 sequence of SEQ ID NO: 16, and optionally, a VH framework 3 sequence of SEQ ID NO:21;
(b) a framework 1 sequence of SEQ ID NO:13, a framework 2 sequence of SEQ ID NO: 17, and optionally, a VH framework 3 sequence of SEQ ID NO:21;
(c) a framework 1 sequence of SEQ ID NO: 13, a framework 2 sequence of SEQ ID NO: 18, and optionally, a VH framework 3 sequence of SEQ ID NO:21;
(d) a framework 1 sequence of SEQ ID NO:13, a framework 2 sequence of SEQ ID NO: 19, and optionally, a VH framework 3 sequence of SEQ ID NO:21;
(e) a framework 1 sequence of SEQ ID NO:14, a framework 2 sequence of SEQ ID NO: 16, and optionally, a VH framework 3 sequence of SEQ ID NO:21;
(f) a framework 1 sequence of SEQ ID NO: 14, a framework 2 sequence of SEQ ID NO: 17, and optionally, a VH framework 3 sequence of SEQ ID NO:21; and
(g) a framework 1 sequence of SEQ ID NO: 14, a framework 2 sequence of SEQ ID NO: 18, and optionally, a VH framework 3 sequence of SEQ ID NO:21.

3. (canceled)

4. The Gmd-binding antibody of claim 1 which comprises a VH of SEQ ID NO: 1-7, or 8, or a VH and a VL pair having a VH of SEQ ID NO: 1-7 or 8 and a VL of SEQ ID NO:33-39, or 40.

5. The Gmd-binding antibody of claim 1 which comprises VL framework sequences selected from the group consisting of:
(a) a framework 1 sequence of SEQ ID NO:45 and a framework 2 sequence of SEQ ID NO:49;
(b) a framework 1 sequence of SEQ ID NO:46 and a framework 2 sequence of SEQ ID NO:50;
(c) a framework 1 sequence of SEQ ID NO:47 and a framework 2 sequence of SEQ ID NO:50;
(d) a framework 1 sequence of SEQ ID NO:47 and a framework 2 sequence of SEQ ID NO:51;
(e) a framework 2 sequence of SEQ ID NO:49 and a framework 3 sequence of SEQ ID NO:53;
(f) a framework 2 sequence of SEQ ID NO:49 and a framework 3 sequence of SEQ ID NO:55;
(g) a framework 2 sequence of SEQ ID NO:50 and a framework 3 sequence of SEQ ID NO:54;
(h) a framework 2 sequence of SEQ ID NO:50 and a framework 3 sequence of SEQ ID NO:55;
(i) a framework 2 sequence of SEQ ID NO:50 and a framework 3 sequence of SEQ ID NO:56;
(j) a framework 2 sequence of SEQ ID NO:50 and a framework 3 sequence of SEQ ID NO:57; and
(k) a framework 2 sequence of SEQ ID NO:51 and a framework 3 sequence of SEQ ID NO:58.

6. (canceled)

7. A Gmd-binding antibody which comprises,
a VH having at least 90%, 95%, 96%, 97%, 98%, 99%, or 100% sequence identity to SEQ ID NO: 1-7 or 8 and a VL having at least 90%, 95%, 96%, 97%, 98%, 99%, or 100% sequence identity to SEQ ID NO:33-39, or 40; and wherein the Gmd-binding antibody does not comprise the VH sequence of SEQ ID NO:9, the VL sequence of SEQ ID NO:41, or the VH sequence of SEQ ID NO:9 and the VL sequence of SEQ ID NO:41, or
a VH and a VL pair selected from the group consisting of:
(a) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:1, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 34 or 35;
(b) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:2, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 34, 35, or 37;
(c) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:3, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33, 35, 36, 38, or 39;
(d) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:4, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 39;
(e) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:5, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 35, 36, 38, or 39;
(f) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:6, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 35, 36, or 38;
(g) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:7, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 34 or 35; and
(h) a VH sequence having a total of 1, 2, 3, 4, 5, 6, 7, 8, 9, 10, fewer than 15, or zero, amino acid substitutions, deletions, and/or insertions from a reference VH sequence of SEQ ID NO:8, and a VL sequence having a total of 1, 2, 3, 4, 5, 6, 7, 8, 9, 10, fewer than 15, or zero, amino acid substitutions, deletions, and/or insertions from a reference VL sequence of SEQ ID NO: 33-38, or 40; and wherein the Gmd-binding antibody does not comprise the VH sequence of SEQ ID NO:9, the VL sequence of SEQ ID NO:41, or the VH sequence of SEQ ID NO:9 and the VL sequence of SEQ ID NO:41.

8. (canceled)

9. (canceled)

10. The Gmd-binding antibody of claim 4 which comprises a VH and a VL pair selected from the group consisting of
(a) a VH sequence of SEQ ID NO: 1 and a VL sequence of SEQ ID NO:34;
(b) a VH sequence of SEQ ID NO:1 and a VL sequence of SEQ ID NO:35;
(c) a VH sequence of SEQ ID NO:2 and a VL sequence of SEQ ID NO:34;
(d) a VH sequence of SEQ ID NO:2 and a VL sequence of SEQ ID NO:35;
(e) a VH sequence of SEQ ID NO:3 and a VL sequence of SEQ ID NO:33;
(f) a VH sequence of SEQ ID NO:3 and a VL sequence of SEQ ID NO:35;
(g) a VH sequence of SEQ ID NO:3, and a VL sequence of SEQ ID NO:36;
(h) a VH sequence of SEQ ID NO:3 and a VL sequence of SEQ ID NO:38;
(i) a VH sequence of SEQ ID NO:3 and a VL sequence of SEQ ID NO:39;
(j) a VH sequence of SEQ ID NO:4 and a VL sequence of SEQ ID NO:39;
(k) a VH sequence of SEQ ID NO:5 and a VL sequence of SEQ ID NO:35;
(l) a VH sequence of SEQ ID NO:5 and a VL sequence of SEQ ID NO:36;
(m) a VH sequence of SEQ ID NO:5 and a VL sequence of SEQ ID NO:38;
(n) a VH sequence of SEQ ID NO:5 and a VL sequence of SEQ ID NO:39;
(o) a VH sequence of SEQ ID NO:6, and a VL sequence of SEQ ID NO:35;
(p) a VH sequence of SEQ ID NO:6 and a VL sequence of SEQ ID NO:36;
(q) a VH sequence of SEQ ID NO:6 and a VL sequence of SEQ ID NO:38;
(r) a VH sequence of SEQ ID NO:7 and a VL sequence of SEQ ID NO:34;
(s) a VH sequence of SEQ ID NO:7 and a VL sequence of SEQ ID NO:35;
(t) a VH sequence of SEQ ID NO:8 and a VL sequence of SEQ ID NO:33;
(u) a VH sequence of SEQ ID NO:8 and a VL sequence of SEQ ID NO:34;
(v) a VH sequence of SEQ ID NO:8 and a VL sequence of SEQ ID NO:35;
(w) a VH sequence of SEQ ID NO:8, and a VL sequence of SEQ ID NO:36;
(x) a VH sequence of SEQ ID NO:8 and a VL sequence of SEQ ID NO:37;
(y) a VH sequence of SEQ ID NO:8 and a VL sequence of SEQ ID NO:38; and
(z) a VH sequence of SEQ ID NO:8 and a VL sequence of SEQ ID NO:40.

11. (canceled)

12. (canceled)

13. The Gmd-binding antibody of claim 1, wherein the Gmd-binding antibody further comprises a heavy chain immunoglobulin constant domain selected from the group consisting of:
(a) a human IgA constant domain;
(b) a human IgD constant domain;
(c) a human IgE constant domain;
(d) a human IgG1 constant domain;
(e) a human IgG2 constant domain;
(f) a human IgG3 constant domain;
(g) a human IgG4 constant domain; and
(h) a human IgM constant domain;
optionally, wherein the constant domain does not contain a carboxy terminal lysine residue, optionally, wherein the Gmd-binding antibody further comprises a light chain immunoglobulin constant domain selected from the group consisting of a human Ig kappa constant domain; and a human Ig lambda constant domain.

14.-16. (canceled)

17. The Gmd-binding antibody of claim 1, wherein the Gmd-binding antibody has at least one characteristic selected from the group consisting of:
(a) inhibits in vitro growth of S. aureus;
(b) inhibits in vivo growth of S. aureus;
(c) promotes cell-independent lysis of S. aureus;
(d) promotes clumping (clustering) of S. aureus;
(e) decreases binary fission of S. aureus;
(f) binds to a polypeptide consisting of the amino acid sequence of SEQ ID NO:72 with a KD of ≤1 nM and ≥1 pM as determined by surface plasmon resonance analysis; and
(g) has antibody dependent cellular cytotoxicity (ADCC).

18. The Gmd-binding antibody of claim 17, wherein the Gmd-binding antibody
(a) inhibits in vitro growth of S. aureus by 20% to 100%, 30% to 100%, 40% to 100%, 50% to 100%, 60% to 100%, 70% to 100%, or 80% to 100%;
(b) inhibits in vivo growth of S. aureus by 20% to 100%, 30% to 100%, 40% to 100%, 50% to 100%, 60% to 100%, 70% to 100%, or 80% to 100%;
(c) promotes cell-independent lysis of S. aureus; or
(d) binds to a polypeptide consisting of the amino acid sequence of SEQ ID NO:72 with a KD of ≤1 nM and ≥1 pM.

19.-21. (canceled)

22. The Gmd-binding antibody of claim 1, wherein the Gmd-binding antibody is a full-length antibody, a humanized antibody, a monoclonal antibody, a recombinant antibody, a chimeric antibody, a bi-specific antibody, a multi-specific antibody, a full-length antibody, or a Gmd-binding antibody fragment.

23. (canceled)

24. The Gmd-binding antibody of claim 22, which is a Gmd-binding fragment selected from the group consisting of a Fab fragment, a Fab′ fragment, a F(ab′)₂ fragment, a Fv fragment, a diabody, and a single chain antibody molecule.

25. An isolated nucleic acid molecule or set of nucleic acid molecules encoding the Gmd-binding antibody of claim 1.

26.-28. (canceled)

29. A vector comprising the nucleic acid molecule or set of nucleic acid molecules of claim 25.

30. A host cell comprising the nucleic acid molecule or set of nucleic acid molecules of claim 25.

31. (canceled)

32. (canceled)

33. A method of making the Gmd-binding antibody of claim 1, comprising culturing the host cell of claim 30 under suitable conditions for producing the Gmd-binding antibody.

34. (canceled)

35. (canceled)

36. A pharmaceutical composition comprising the Gmd-binding antibody of claim 1 and a pharmaceutically acceptable carrier.

37.-44. (canceled)

45. A method for treating a Staphylococcus infection or a Staphylococcus infection associated condition, comprising administering to a subject having or at risk of having an Staphylococcus infection, an effective amount of the antibody of claim 1.

46. The method of claim 45, wherein the Staphylococcus infection is an S. aureus infection, optionally, wherein the S. aureus is resistant to an antibiotic, or wherein the S. aureus is methicillin-resistant (MRSA).

47.-52. (canceled)


SEQUENCE LISTING (75 sequences)

SEQ ID NO: 1 - protein, 122 aa, Artificial Sequence
  Synthetic Polypeptide
QVQLVQSGAEVKKPGASVKVSCKASGYTFTTYGMSWIRQSPSRGLEWMGWINTYSGVPTYADDFKGRFVFSLDTSVSTAYLQICSLKAEDTAVYYCAREEYSSGYAAWFPYWGQGTLVTVSS

SEQ ID NO: 2 - protein, 122 aa, Artificial Sequence
  Synthetic Polypeptide
QVQLVQSGAEVKKPGASVKVSCKASGYTFTTYGMSWIRQSPSRGLEWLGWINTYSGVPTYADDFKGRFVFSLDTSVSTAYLQICSLKAEDTAVYYCAREEYSSGYAAWFPYWGQGTLVTVSS

SEQ ID NO: 3 - protein, 122 aa, Artificial Sequence
  Synthetic Polypeptide
EVQLVQSGAEVKKPGESLRISCKGSGYTFTTYGMSWIRQSPSRGLEWLGWINTYSGVPTYADDFKGRFVFSLDTSVSTAYLQICSLKAEDTAVYYCAREEYSSGYAAWFPYWGQGTLVTVSS

SEQ ID NO: 4 - protein, 122 aa, Artificial Sequence
  Synthetic Polypeptide
EVQLVQSGAEVKKPGESLRISCKGSGYTFTTYGMSWVRQARGQRLEWIGWINTYSGVPTYADDFKGRFVFSLDTSVSTAYLQICSLKAEDTAVYYCAREEYSSGYAAWFPYWGQGTLVTVSS

SEQ ID NO: 5 - protein, 122 aa, Artificial Sequence
  Synthetic Polypeptide
EVQLVQSGAEVKKPGESLRISCKGSGYTFTTYGMSWVRQATGQGLEWMGWINTYSGVPTYADDFKGRFVFSLDTSVSTAYLQICSLKAEDTAVYYCAREEYSSGYAAWFPYWGQGTLVTVSS

SEQ ID NO: 6 - protein, 122 aa, Artificial Sequence
  Synthetic Polypeptide
QVQLVQSGAEVKKPGASVKVSCKASGYTFTTYGMSWVRQARGQRLEWIGWINTYSGVPTYADDFKGRFVFSLDTSVSTAYLQICSLKAEDTAVYYCAREEYSSGYAAWFPYWGQGTLVTVSS

SEQ ID NO: 7 - protein, 122 aa, Artificial Sequence
  Synthetic Polypeptide
QVQLVQSGAEVKKPGASVKVSCKASGYTFTTYGMSWVRQATGQGLEWMGWINTYSGVPTYADDFKGRFVFSLDTSVSTAYLQICSLKAEDTAVYYCAREEYSSGYAAWFPYWGQGTLVTVSS

SEQ ID NO: 8 - protein, 122 aa, Artificial Sequence
  Synthetic Polypeptide
QVQLVQSGAEVKKPGASVKVSCKASGYTFTTYGMSWVRQAPGQGLEWMGWINTYSGVPTYADDFKGRFVFSLDTSVSTAYLQICSLKAEDTAVYYCAREEYSSGYAAWFPYWGQGTLVTVSS

SEQ ID NO: 9 - protein, 122 aa, Artificial Sequence
  Synthetic Polypeptide
QIQLVQSGPELKKPGETVKISCKASGYTFTTYGMSWVNQAPGKGLKWMGWINTYSGVPTYADDFKGRFVFSLETSASTAYLQINNLKNEDTATYFCAREEYSSGYAAWFPYWGQGTLVTVSS

SEQ ID NO: 10 - protein, 5 aa, Artificial Sequence
  Synthetic Polypeptide
SYAIS

SEQ ID NO: 11 - protein, 17 aa, Artificial Sequence
  Synthetic Polypeptide
GIIPIFGTASYAQKFQG

SEQ ID NO: 12 - protein, 12 aa, Artificial Sequence
  Synthetic Polypeptide
TPYYDSSGYLDV

SEQ ID NO: 13 - protein, 30 aa, Artificial Sequence
  Synthetic Polypeptide
QVQLVQSGAEVKKPGASVKVSCKASGYTFT

SEQ ID NO: 14 - protein, 30 aa, Artificial Sequence
  Synthetic Polypeptide
EVQLVQSGAEVKKPGESLRISCKGSGYTFT

SEQ ID NO: 15 - protein, 30 aa, Artificial Sequence
  Synthetic Polypeptide
QIQLVQSGPELKKPGETVKISCKASGYTFT

SEQ ID NO: 16 - protein, 14 aa, Artificial Sequence
  Synthetic Polypeptide
WIRQSPSRGLEWLG

SEQ ID NO: 17 - protein, 14 aa, Artificial Sequence
  Synthetic Polypeptide
WVRQARGQRLEWIG

SEQ ID NO: 18 - protein, 14 aa, Artificial Sequence
  Synthetic Polypeptide
WVRQATGQGLEWMG

SEQ ID NO: 19 - protein, 14 aa, Artificial Sequence
  Synthetic Polypeptide
WVRQAPGQGLEWMG

SEQ ID NO: 20 - protein, 14 aa, Artificial Sequence
  Synthetic Polypeptide
WVNQAPGKGLKWMG

SEQ ID NO: 21 - protein, 31 aa, Artificial Sequence
  Synthetic Polypeptide
FVFSLDTSVSTAYLQICSLKAEDTAVYYCAR

SEQ ID NO: 22 - protein, 32 aa, Artificial Sequence
  Synthetic Polypeptide
RFVFSLETSASTAYLQINNLKNEDTATYFCAR

SEQ ID NO: 23 - protein, 11 aa, Artificial Sequence
  Synthetic Polypeptide
WGQGTLVTVSS

SEQ ID NO: 24 - DNA, 366 bp, Artificial Sequence
  Synthetic Polynucleotide
caggttcagctggtgcagtctggagctgaggtgaagaagcctggggcctcagtgaaggtctcctgcaaggcttctgggtataccttcacaacgtatggaatgagctggatcaggcagtccccatcgagaggccttgagtggatgggttggataaacacctactctggagtgccaacatatgctgatgacttcaagggaagatttgtcttctccttggacacctctgtcagcacggcatatctgcagatctgcagcctaaaggctgaggacactgccgtgtattactgtgcaagagaggagtacagctcaggctacgcggcctggtttccttactggggccagggaacgctggtcaccgtcagctca

SEQ ID NO: 25 - DNA, 366 bp, Artificial Sequence
  Synthetic Polynucleotide
caggttcagctggtgcagtctggagctgaggtgaagaagcctggggcctcagtgaaggtctcctgcaaggcttctgggtataccttcacaacgtatggaatgagctggatcaggcagtccccatcgagaggccttgagtggctgggttggataaacacctactctggagtgccaacatatgctgatgacttcaagggaagatttgtcttctccttggacacctctgtcagcacggcatatctgcagatctgcagcctaaaggcagaggacactgccgtgtattactgtgcaagagaggagtacagctcaggctacgcggcctggtttccttactggggccagggaacgctggtcaccgtcagctca

SEQ ID NO: 26 - DNA, 366 bp, Artificial Sequence
  Synthetic Polynucleotide
gaagtgcagctggtgcagtctggagcagaggtgaaaaagcccggggagtctctgaggatctcctgtaagggttctgggtataccttcacaacgtatggaatgagctggatcaggcagtccccatcgagaggccttgagtggctgggttggataaacacctactctggagtgccaacatatgctgatgacttcaagggaagatttgtcttctccttggacacctctgtcagcacggcatatctgcagatctgcagcctaaaggctgaggacactgccgtgtattactgtgcaagagaggagtacagctcaggctacgcggcctggtttccttactggggccagggaacgctggtcaccgtcagctca

SEQ ID NO: 27 - DNA, 366 bp, Artificial Sequence
  Synthetic Polynucleotide
gaagtgcagctggtgcagtctggagcagaggtgaaaaagcccggggagtctctgaggatctcctgtaagggttctgggtataccttcacaacgtatggaatgagctgggtgcgacaggctcgtggacaacgccttgagtggataggttggataaacacctactctggagtgccaacatatgctgatgacttcaagggaagatttgtcttctccttggacacctctgtcagcacggcatatctgcagatctgcagcctaaaggctgaggacactgccgtgtattactgtgcaagagaggagtacagctcaggctacgcggcctggtttccttactggggccagggaacgctggtcaccgtcagctca

SEQ ID NO: 28 - DNA, 366 bp, Artificial Sequence
  Synthetic Polynucleotide
gaagtgcagctggtgcagtctggagcagaggtgaaaaagcccggggagtctctgaggatctcctgtaagggttctgggtataccttcacaacgtatggaatgagctgggtgcgacaggccactggacaagggcttgagtggatgggttggataaacacctactctggagtgccaacatatgctgatgacttcaagggaagatttgtcttctccttggacacctctgtcagcacggcatatctgcagatctgcagcctaaaggctgaggacactgccgtgtattactgtgcaagagaggagtacagctcaggctacgcggcctggtttccttactggggccagggaacgctggtcaccgtcagctca

SEQ ID NO: 29 - DNA, 366 bp, Artificial Sequence
  Synthetic Polynucleotide
caggttcagctggtgcagtctggagctgaggtgaagaagcctggggcctcagtgaaggtctcctgcaaggcttctgggtataccttcacaacgtatggaatgagctgggtgcgacaggcccgtggacaacgccttgagtggataggttggataaacacctactctggagtgccaacatatgctgatgacttcaagggaagatttgtcttctccttggacacctctgtcagcacggcatatctgcagatctgcagcctaaaggctgaggacactgccgtgtattactgtgcaagagaggagtacagctcaggctacgcggcctggtttccttactggggccagggaacgctggtcaccgtcagctca

SEQ ID NO: 30 - DNA, 366 bp, Artificial Sequence
  Synthetic Polynucleotide
caggttcagctggtgcagtctggagctgaggtgaagaagcctggggcctcagtgaaggtctcctgcaaggcttctgggtataccttcacaacgtatggaatgagctgggtgcgacaggccactggacaagggcttgagtggatgggttggataaacacctactctggagtgccaacatatgctgatgacttcaagggaagatttgtcttctccttggacacctctgtcagcacggcatatctgcagatctgcagcctaaaggctgaggacactgccgtgtattactgtgcaagagaggagtacagctcaggctacgcggcctggtttccttactggggccagggaacgctggtcaccgtcagctca

SEQ ID NO: 31 - DNA, 366 bp, Artificial Sequence
  Synthetic Polynucleotide
caggttcagctggtgcagtctggagctgaggtgaagaagcctggggcctcagtgaaggtctcctgcaaggcttctgggtataccttcacaacgtatggaatgagctgggtgcgacaggcccctggacaagggcttgagtggatgggttggataaacacctactctggagtgccaacatatgctgatgacttcaagggaagatttgtcttctccttggacacctctgtcagcacggcatatctgcagatctgcagcctaaaggcagaggacactgccgtgtattactgtgcaagagaggagtacagctcaggctacgcggcctggtttccttactggggccagggaacgctggtcaccgtcagctca

SEQ ID NO: 32 - DNA, 366 bp, Artificial Sequence
  Synthetic Polynucleotide
cagatccagttggtacagtctggacctgagctgaagaagcctggagagacagtcaagatctcctgcaaggcttctgggtataccttcacaacgtatggaatgagctgggtgaatcaggctccaggaaagggtttaaagtggatgggctggataaacacctactctggagtgccaacatatgctgatgacttcaagggacggtttgtcttctctttggaaacctctgccagcactgcctatttgcagatcaacaacctcaaaaatgaggacacggctacatatttctgtgcaagagaggagtacagctcaggctacgcggcctggtttccttactggggccagggaacgctggtcaccgtcagctca

SEQ ID NO: 33 - protein, 107 aa, Artificial Sequence
  Synthetic Polypeptide
EIVLTQSPDFQSVTPKEKVTITCRASQSISNNLHWYQQKPGKAPKLLIYYASRSISGIPPRFSGSGYGTDFTLTINNIESEDAAYYFCQQSNSWPLTFGQGTKVEIK

SEQ ID NO: 34 - protein, 107 aa, Artificial Sequence
  Synthetic Polypeptide
AIQLTQSPSSLSASVGDRVTITCRASQSISNNLHWYQQKPGQAPRLLIYYASRSISGVPSRFSGSGSGTDFTFTISSLQPEDIATYYCQQSNSWPLTFGQGTKVEIK

SEQ ID NO: 35 - protein, 107 aa, Artificial Sequence
  Synthetic Polypeptide
AIQLTQSPSSLSASVGDRVTITCRASQSISNNLHWYQQKPGKAPKLLIYYASRSISGVPSRFSGSGSGTEFTLTISSLQPDDFATYYCQQSNSWPLTFGQGTKVEIK

SEQ ID NO: 36 - protein, 107 aa, Artificial Sequence
  Synthetic Polypeptide
AIQLTQSPSSLSASVGDRVTITCRASQSISNNLHWYQQKPGQAPRLLIYYASRSISGVPSRFSGSGSGTEFTLTISSLQPDDFATYYCQQSNSWPLTFGQGTKVEIK

SEQ ID NO: 37 - protein, 107 aa, Artificial Sequence
  Synthetic Polypeptide
EIVLTQSPATLSLSPGERATLSCRASQSISNNLHWYQQKPGQAPRLLIYYASRSISGVPSRFSGSGSGTDFTFTISSLEAEDAATYYCQQSNSWPLTFGQGTKVEIK

SEQ ID NO: 38 - protein, 107 aa, Artificial Sequence
  Synthetic Polypeptide
EIVLTQSPATLSLSPGERATLSCRASQSISNNLHWYQQKPGQAPRLLIYYASRSISGIPARFSGSGSGTEFTLTISSLQSEDFAVYYCQQSNSWPLTFGQGTKVEIK

SEQ ID NO: 39 - protein, 107 aa, Artificial Sequence
  Synthetic Polypeptide
EIVLTQSPATLSLSPGERATLSCRASQSISNNLHWYLQKPGQSPQLLIYYASRSISGVPDRFSGSGSGTDFTLKISRVEAEDVGVYYCQQSNSWPLTFGQGTKVEIK

SEQ ID NO: 40 - protein, 107 aa, Artificial Sequence
  Synthetic Polypeptide
EIVLTQSPDFQSVTPKEKVTITCRASQSISNNLHWYLQKPGQSPQLLIYYASRSISGVPDRFSGSGSGTDFTLKISRVEAEDVGVYYCQQSNSWPLTFGQGTKVEIK

SEQ ID NO: 41 - protein, 107 aa, Artificial Sequence
  Synthetic Polypeptide
DIVLTQSPATLSVTPGDSVSLSCRASQSISNNLHWYQQKSHESPRLLIEYASRSISGIPSRFSGGGSGTDFTLSINSVESEDFGLYFCQQSNSWPLTFGQGTKVEIK

SEQ ID NO: 42 - protein, 11 aa, Artificial Sequence
  Synthetic Polypeptide
RASQSISNNLH

SEQ ID NO: 43 - protein, 7 aa, Artificial Sequence
  Synthetic Polypeptide
YASRSIS

SEQ ID NO: 44 - protein, 9 aa, Artificial Sequence
  Synthetic Polypeptide
QQSNSWPLT

SEQ ID NO: 45 - protein, 23 aa, Artificial Sequence
  Synthetic Polypeptide
EIVLTQSPDFQSVTPKEKVTITC

SEQ ID NO: 46 - protein, 23 aa, Artificial Sequence
  Synthetic Polypeptide
EIVLTQSPDFQSVTPKEKVTITC

SEQ ID NO: 47 - protein, 23 aa, Artificial Sequence
  Synthetic Polypeptide
AIQLTQSPSSLSASVGDRVTITC

SEQ ID NO: 48 - protein, 23 aa, Artificial Sequence
  Synthetic Polypeptide
EIVLTQSPATLSLSPGERATLSC

SEQ ID NO: 49 - protein, 22 aa, Artificial Sequence
  Synthetic Polypeptide
IVLTQSPATLSVTPGDSVSLSC

SEQ ID NO: 50 - protein, 15 aa, Artificial Sequence
  Synthetic Polypeptide
WYQQKPGKAPKLLIY

SEQ ID NO: 51 - protein, 15 aa, Artificial Sequence
  Synthetic Polypeptide
WYQQKPGQAPRLLIY

SEQ ID NO: 52 - protein, 15 aa, Artificial Sequence
  Synthetic Polypeptide
WYLQKPGQSPQLLIY

SEQ ID NO: 53 - protein, 15 aa, Artificial Sequence
  Synthetic Polypeptide
WYQQKSHESPRLLIE

SEQ ID NO: 54 - protein, 32 aa, Artificial Sequence
  Synthetic Polypeptide
GIPPRFSGSGYGTDFTLTINNIESEDAAYYFC

SEQ ID NO: 55 - protein, 32 aa, Artificial Sequence
  Synthetic Polypeptide
GVPSRFSGSGSGTDFTFTISSLQPEDIATYYC

SEQ ID NO: 56 - protein, 32 aa, Artificial Sequence
  Synthetic Polypeptide
GVPSRFSGSGSGTEFTLTISSLQPDDFATYYC

SEQ ID NO: 57 - protein, 32 aa, Artificial Sequence
  Synthetic Polypeptide
GVPSRFSGSGSGTDFTFTISSLEAEDAATYYC

SEQ ID NO: 58 - protein, 32 aa, Artificial Sequence
  Synthetic Polypeptide
GIPARFSGSGSGTEFTLTISSLQSEDFAVYYC

SEQ ID NO: 59 - protein, 32 aa, Artificial Sequence
  Synthetic Polypeptide
GVPDRFSGSGSGTDFTLKISRVEAEDVGVYYC

SEQ ID NO: 60 - protein, 32 aa, Artificial Sequence; identical to SEQ ID NO: 59
  Synthetic Polypeptide

SEQ ID NO: 61 - protein, 32 aa, Artificial Sequence
  Synthetic Polypeptide
GIPSRFSGGGSGTDFTLSINSVESEDFGLYFC

SEQ ID NO: 62 - protein, 10 aa, Artificial Sequence
  Synthetic Polypeptide
FGQGTKVEIK

SEQ ID NO: 63 - DNA, 321 bp, Artificial Sequence
  Synthetic Polypeptide
gaaattgtgctgactcagtctccagactttcagtctgtgactccaaaggagaaagtcaccatcacctgcagggccagccaaagtattagcaacaacctacactggtatcagcagaaaccagggaaagctcctaagctcctgatctattatgcttcccggtccatctctgggatcccacctcgattcagtggcagcgggtatggaacagattttaccctcacaattaataacatagaatctgaggatgctgcatattacttctgtcaacagagtaacagctggccgctcacgttcggccaagggaccaaggtggaaatcaaa

SEQ ID NO: 64 - DNA, 321 bp, Artificial Sequence
  Synthetic Polypeptide
gccatccagttgacccagtctccatcctccctgtctgcatctgtaggagacagagtcaccatcacttgcagggccagccaaagtattagcaacaacctacactggtaccagcagaaacctggccaggctcccaggctcctcatctattatgcttcccggtccatctctggggtcccatcaaggttcagtggaagtggatctgggacagattttactttcaccatcagcagcctgcagcctgaagatattgcaacatattactgtcaacagagtaacagctggccgctcacgttcggccaagggaccaaggtggaaatcaaa

SEQ ID NO: 65 - DNA, 321 bp, Artificial Sequence
  Synthetic Polypeptide
gccatccagttgacccagtctccatcctccctgtctgcatctgtaggagacagagtcaccatcacttgcagggccagccaaagtattagcaacaacctacactggtatcagcagaaaccagggaaagctcctaagctcctgatctattatgcttcccggtccatctctggggtcccatcaaggttcagcggcagtggatctgggacagaattcactctcaccatcagcagcctgcagcctgatgattttgcaacttattactgtcaacagagtaacagctggccgctcacgttcggccaagggaccaaggtggaaatcaaa

SEQ ID NO: 66 - DNA, 321 bp, Artificial Sequence
  Synthetic Polypeptide
gccatccagttgacccagtctccatcctccctgtctgcatctgtaggagacagagtcaccatcacttgcagggccagccaaagtattagcaacaacctacactggtaccagcagaaacctggccaggctcccaggctcctcatctattatgcttcccggtccatctctggggtcccatcaaggttcagcggcagtggatctgggacagaattcactctcaccatcagcagcctgcagcctgatgattttgcaacttattactgtcaacagagtaacagctggccgctcacgttcggccaagggaccaaggtggaaatcaaa

SEQ ID NO: 67 - DNA, 321 bp, Artificial Sequence
  Synthetic Polypeptide
gaaattgtgttgacacagtctccagccaccctgtctttgtctccaggggaaagagccaccctctcctgcagggccagccaaagtattagcaacaacctacactggtaccagcagaaacctggccaggctcccaggctcctcatctattatgcttcccggtccatctctggggtcccctcgaggttcagtggcagtggatctgggacagatttcacctttaccatcagtagcctggaagctgaagatgctgcaacatattactgtcaacagagtaacagctggccgctcacgttcggccaagggaccaaggtggaaatcaaa

SEQ ID NO: 68 - DNA, 321 bp, Artificial Sequence
  Synthetic Polypeptide
gaaattgtgttgacacagtctccagccaccctgtctttgtctccaggggaaagagccaccctctcctgcagggccagccaaagtattagcaacaacctacactggtaccagcagaaacctggccaggctcccaggctcctcatctattatgcttcccggtccatctctgggatcccagccaggttcagtggcagtgggtctgggacagagttcactctcaccatcagcagcctgcagtctgaagattttgcagtttattactgtcaacagagtaacagctggccgctcacgttcggccaagggaccaaggtggaaatcaaa

SEQ ID NO: 69 - DNA, 321 bp, Artificial Sequence
  Synthetic Polypeptide
gaaattgtgttgacacagtctccagccaccctgtctttgtctccaggggaaagagccaccctctcctgcagggccagccaaagtattagcaacaacctacactggtacctgcagaagccagggcagtctccacagctcctgatctattatgcttcccggtccatctctggggtcccagacaggttcagtggcagtgggtcaggcactgatttcacactgaaaatcagcagggtggaggctgaggatgttggagtttattactgtcaacagagtaacagctggccgctcacgttcggccaagggaccaaggtggaaatcaaa

SEQ ID NO: 70 - DNA, 321 bp, Artificial Sequence
  Synthetic Polypeptide
gaaattgtgctgactcagtctccagactttcagtctgtgactccaaaggagaaagtcaccatcacctgcagggccagccaaagtattagcaacaacctacactggtacctgcagaagccagggcagtctccacagctcctgatctattatgcttcccggtccatctctggggtcccagacaggttcagtggcagtgggtcaggcactgatttcacactgaaaatcagcagggtggaggctgaggatgttggagtttattactgtcaacagagtaacagctggccgctcacgttcggccaagggaccaaggtggaaatcaaa

SEQ ID NO: 71 - DNA, 321 bp, Artificial Sequence
  Synthetic Polypeptide
gatattgtgctaactcagtctccagccaccctgtctgtgactccaggagatagcgtcagtctttcctgcagggccagccaaagtattagcaacaacctacactggtatcaacaaaaatcacatgagtctccaaggcttctcatcgaatatgcttcccggtccatctctgggatcccctctaggttcagtggcggtggatcagggacagatttcactctcagtatcaacagtgtggagtctgaagattttggattgtatttctgtcaacagagtaacagctggccgctcacgttcggccaagggaccaaggtggaaatcaaa

SEQ ID NO: 72 - DNA, 1425 bp, Artificial Sequence
  Synthetic Polypeptide
atggacatgagggtccccgctcagctcctggggctcctgctgctctggctcccaggtgccaagtgtcagatccagttggtacagtctggacctgagctgaagaagcctggagagacagtcaagatctcctgcaaggcttctgggtataccttcacaacgtatggaatgagctgggtgaatcaggctccaggaaagggtttaaagtggatgggctggataaacacctactctggagtgccaacatatgctgatgacttcaagggacggtttgtcttctctttggaaacctctgccagcactgcctatttgcagatcaacaacctcaaaaatgaggacacggctacatatttctgtgcaagagaggagtacagctcaggctacgcggcctggtttccttactggggccagggaacgctggtcaccgtcagctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtcagcaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 73 - DNA, 711 bp, Artificial Sequence
  Synthetic Polypeptide
atggacatgagggtccccgctcagctcctggggctcctgctgctctggctcccaggtgccaagtgtgatattgtgctaactcagtctccagccaccctgtctgtgactccaggagatagcgtcagtctttcctgcagggccagccaaagtattagcaacaacctacactggtatcaacaaaaatcacatgagtctccaaggcttctcatcgaatatgcttcccggtccatctctgggatcccctctaggttcagtggcggtggatcagggacagatttcactctcagtatcaacagtgtggagtctgaagattttggattgtatttctgtcaacagagtaacagctggccgctcacgttcggccaagggaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 74 - protein, 236 aa, Artificial Sequence
  Synthetic Polypeptide
MDMRVPAQLLGLLLLWLPGAKCDIVLTQSPATLSVTPGDSVSLSCRASQSISNNLHWYQQKSHESPRLLIEYASRSISGIPSRFSGGGSGTDFTLSINSVESEDFGLYFCQQSNSWPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 474 aa, Artificial Sequence
  Synthetic Polypeptide
MDMRVPAQLLGLLLLWLPGAKCQIQLVQSGPELKKPGETVKISCKASGYTFTTYGMSWVNQAPGKGLKWMGWINTYSGVPTYADDFKGRFVFSLETSASTAYLQINNLKNEDTATYFCAREEYSSGYAAWFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK